(19) United States
(12) Patent Application Publication
(10) Pub. No.: US 2024/0107794 A1
(43) Pub. Date: Mar. 28, 2024

(54) ORGANIC ELECTROLUMINESCENT DEVICE

(71) Applicant: ROHM AND HAAS ELECTRONIC MATERIALS KOREA LTD., Chungcheongnam-do (KR)

(72) Inventors: Su-Hyun LEE, Gyeonggi-do (KR); Soo-Yong LEE, Gyeonggi-do (KR); Chi-Sik KIM, Gyeonggi-do (KR); HaeYeon KIM, Gyeonggi-do (KR); Yoo-Jin DOH, Gyeonggi-do (KR); SeungAe KIM, Gyeonggi-do (KR); Mi-Ja LEE, Gyeonggi-do (KR)

(21) Appl. No.: 18/457,633

(22) Filed: Aug. 29, 2023

(30) Foreign Application Priority Data
Sep. 5, 2022 (KR) 10-2022-0111937
Aug. 3, 2023 (KR) 10-2023-0101503

(51) Int. Cl. H10K 50/18; C07D 235/18; C09K 11/06; H10K 50/11; H10K 50/16; H10K 85/60

(52) U.S. Cl. CPC H10K 50/18; C07D 235/18; C09K 11/06; H10K 50/11; H10K 50/16; H10K 85/626; H10K 85/6572; H10K 2101/90

(57) ABSTRACT

The present disclosure relates to an organic electroluminescent device. The organic electroluminescent device according to the present disclosure includes a deuterated organic electroluminescent material, so it can exhibit a low driving voltage and high luminous efficiency.

### TECHNICAL FIELD

The present disclosure relates to an organic electroluminescent device.

### BACKGROUND ART

An organic electroluminescent device (OLED) changes electric energy into light by applying electricity to an organic electroluminescent material, and its basic structure was first reported by Eastman Kodak in 1987. The OLED commonly comprises an anode, a cathode, and an organic layer formed between the two electrodes. The organic layer of the OLED may comprise a hole injection layer, a hole transport layer, an electron blocking layer, a light-emitting layer (containing host and dopant materials), an electron buffer layer, a hole blocking layer, an electron transport layer, an electron injection layer, etc. The materials used in the organic layer can be classified into a hole injection material, a hole transport material, an electron blocking material, a light-emitting material, an electron buffer material, a hole blocking material, an electron transport material, an electron injection material, etc., depending on their functions.

In such an OLED, holes from the anode and electrons from the cathode are injected into a light-emitting layer by the application of electric voltage, and excitons having high energy are produced by the recombination of the holes and electrons. The organic light-emitting compound moves into an excited state by the energy and emits light from energy when the organic light-emitting compound returns to the ground state from the excited state.

In recent years, due to the potential of flat panel displays and general lighting devices, the development of new materials for this is continuously required. The development of excellent high-performance materials and more desirable device structures is required in order to improve the performance required in medium and large-sized OLED panels.

Unlike red and green high-efficiency phosphorescent materials which have already been commercialized among the light-emitting materials of the OLED, it has been pointed out that a blue phosphorescent material is not suitable for long-term use such as several years or more, since the blue phosphorescent material has short lifespan and high driving voltage, and thus, a fluorescent material is used. As such, conventional materials have not been able to satisfy the light-emitting characteristics of the OLED, and thus development of an OLED including an organic electroluminescent material having excellent performance is required.

KR Patent Nos. 10-0691543, 10-0998838, 10-2123759, and 10-1942910 discloses an OLED in that an anthracene derivative or a triazine derivative is comprised in a material for an electron injection layer, an electron transport layer, and/or a hole transport auxiliary layer. However, said references do not specifically disclose an OLED including a combination of a deuterated organic electroluminescent materials specified herein.

### DISCLOSURE OF THE INVENTION

### Technical Problem

The object of the present disclosure is firstly, to provide an organic electroluminescent device having low driving voltage and high luminous efficiency.

### Solution to Problems

As a result of intensive studies to solve the technical problem above, the present inventors found that the aforementioned objective can be achieved by an organic electroluminescent device comprising an anode; a cathode; at least one light-emitting layer positioned between the anode and the cathode; and at least one organic layer positioned between the light-emitting layer and the cathode, wherein the organic layer includes at least one deuterated compound, and the light-emitting layer includes a light-emitting compound including an iridium (Ir), platinum (Pt), or boron (B) compound, but does not include a delayed fluorescent compound, so that the present invention was completed.

### Advantageous Effects of Invention

By comprising the deuterated compound according to the present disclosure, an organic electroluminescent device having low driving voltage and high luminous efficiency can be provided.

### BRIEF DESCRIPTION OF THE DRAWINGS

FIG. 1 shows an example of an organic electroluminescent device according to one embodiment of the present disclosure.

FIG. 2 shows an example of an organic electroluminescent device according to another embodiment of the present disclosure.

### MODE FOR THE INVENTION

Hereinafter, the present disclosure will be described in detail. However, the following description is intended to explain the invention, and is not meant in any way to restrict the scope of the invention.

The present disclosure relates to an organic electroluminescent device comprising an anode; a cathode; at least one light-emitting layer positioned between the anode and the cathode; and at least one organic layer positioned between the light-emitting layer and the cathode.

The organic electroluminescent device according to the present disclosure comprises an organic layer including at least one deuterated compound represented by Formula 1 or 2, and a light-emitting layer including a light-emitting compound comprising an iridium (Ir), platinum (Pt), or boron (B) compound, without comprising a delayed fluorescent compound.

The term “organic electroluminescent compound” in the present disclosure means a compound that may be used in an organic electroluminescent device. The organic electroluminescent compound may be comprised in any material layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, an electron injection material, etc.

The “electron transport zone” in the present disclosure means a zone where electrons move between the light-emitting layer and the cathode. For example, the electron transport zone may include at least one of a hole blocking layer, an electron transport layer, and an electron injection layer, preferably an electron transport layer and an electron injection layer. The hole blocking layer serves to prevent holes from entering the cathode through the light-emitting layer in driving the organic electroluminescent device.

The “hole transport zone” in the present disclosure means a zone where holes move between the anode and the light-emitting layer. For example, the hole transport zone may include at least one of a hole injection layer, a hole transport layer, a hole auxiliary layer, a light-emitting auxiliary layer, and an electron blocking layer. Each of the hole injection layer, a hole transport layer, a hole auxiliary layer, a light-emitting auxiliary layer, and an electron blocking layer can be a single layer or a multi-layer of which two or more layers are stacked.

The term “deuteriumization” in the present disclosure means that at least one hydrogen atom of a compound or a functional group is replaced with deuterium, and includes replacement of some or all of the hydrogen atoms with deuterium.

The term “(C1-C30)alkyl” in the present disclosure is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. The term “(C3-C30)cycloalkyl” in the present disclosure is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc.

The term “(C6-C30)aryl(ene)” in the present disclosure is a monocyclic or fused ring radical derived from an aromatic hydrocarbon having 6 to 30 ring backbone carbon atoms, in which the number of the ring backbone carbon atoms is preferably 6 to 20, more preferably 6 to 15, and may be partially saturated. The aryl may comprise a spiro structure. Examples of the aryl specifically include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. The term “(3- to 30-membered)heteroaryl(ene)” in the present disclosure is an aryl having 3 to 30 ring backbone atoms including at least one, preferably 1 to 4 heteroatoms selected from the group consisting of B, N, O, S, Si, P, Se, and Ge, in which the number of ring backbone atoms is preferably 3 to 30, more preferably 5 to 20. The above heteroaryl or heteroarylene may be a monocyclic ring, or a fused ring condensed with at least one benzene ring; and may be partially saturated. Also, the above heteroaryl or heteroarylene in the present disclosure may be one formed by linking at least one heteroaryl or aryl group to a heteroaryl group via a single bond(s) and may comprise a spiro structure. Examples of the heteroaryl specifically may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. The term “a fused ring of (C3-C30) aliphatic ring and (C6-C30) aromatic ring” in the present disclosure means a ring formed by fusing at least one aliphatic ring having 3 to 30 ring backbone carbon atoms in which the number of the carbon atoms is preferably 3 to 25, more preferably 3 to 18, and at least one aromatic ring having 6 to 30 ring backbone carbon atoms in which the number of the carbon atoms is preferably 6 to 25, more preferably 6 to 18. For example, the fused ring may be a fused ring of at least one benzene and at least one cyclohexane, or a fused ring of at least one naphthalene and at least one cyclopentane, etc. The carbon atoms in the fused ring of (C3-C30) aliphatic ring and (C6-C30) aromatic ring of the present disclosure may be replaced with at least one heteroatoms selected from B, N, O, S, Si and P, preferably at least one heteroatoms selected from N, O and S. The term “Halogen” in the present disclosure includes F, Cl, Br, and I.

In addition, “ortho (o)”, “meta (m)”, and “para (p)” in the present disclosure are meant to signify the substitution position of all substituents. Ortho position is a compound with substituents, which are adjacent to each other, i.e., at the 1 and 2 positions on benzene. Meta position is the next substitution position of the immediately adjacent substitution position, i.e., a compound with substituents at the 1 and 3 positions on benzene. Para position is the next substitution position of the meta position, i.e., a compound with substituents at the 1 and 4 positions on benzene.

The term “a ring formed in linking to an adjacent substituent” in the present disclosure means a substituted or unsubstituted (3- to 50-membered) mono- or polycyclic, alicyclic, aromatic ring, or a combination thereof, formed by linking or fusing two or more adjacent substituents, preferably, may be a substituted or unsubstituted (5- to 40-membered) mono- or polycyclic, alicyclic, aromatic ring, or a combination thereof. Further, the formed ring may include at least one heteroatom selected from the group consisting of B, N, O, S, Si and P, preferably, at least one heteroatom selected from N, O and S. According to one embodiment of the present disclosure, the number of atoms in the ring skeleton is 5 to 35; according to another embodiment of the present disclosure, the number of atoms in the ring skeleton is 5 to 30. In one embodiment, the fused ring may be, for example, a substituted or unsubstituted fluorene ring, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted benzofluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, “substituted” in the expression “a substituted or unsubstituted” means that a hydrogen atom in a certain functional group is replaced with another atom or functional group, i.e., a substituent, and substituted with a group to which two or more substituents are connected among the substituents. For example, “a substituent to which two or more substituents are connected” may be pyridine-triazine. That is, pyridine-triazine may be heteroaryl or may be interpreted as a substituent in which two heteroaryls are connected. Preferably, the substituents of the substituted alkyl, the substituted alkenyl, the substituted aryl(ene), the substituted heteroaryl(ene), the substituted cycloalkyl, the substituted alkoxy, the substituted trialkylsilyl, the substituted dialkylarylsilyl, the substituted alkyldiarylsilyl, the substituted triarylsilyl, or the substituted fused ring of aliphatic ring and aromatic ring in the formulas of the present disclosure, each independently are at least one selected from the group consisting of deuterium; halogen; cyano; carboxyl; nitro; hydroxyl; phosphine oxide; (C1-C30)alkyl, halo(C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3- to 7-membered)heterocycloalkyl, (C6-C30)aryloxy, (C6-C30)arylthio, (5- to 30-membered)heteroaryl unsubstituted or substituted with (C6-C30)aryl, (C6-C30)aryl unsubstituted or substituted with (5- to 30-membered)heteroaryl, tri(C1-C30)alkylsilyl, tri(C6-C30)arylsilyl, di(C1-C30)alkyl(C6-C30)arylsilyl, (C1-C30)alkyldi(C6-C30)arylsilyl, amino, mono- or di-(C1-C30)alkylamino, mono- or di-(C6-C30)arylamino unsubstituted or substituted with (C1-C30)alkyl, (C1-C30)alkyl(C6-C30)arylamino, (C1-C30)alkylcarbonyl, (C1-C30)alkoxycarbonyl, (C6-C30)arylcarbonyl, (C6-C30)arylphosphinyl, di(C6-C30)arylboronyl, di(C1-C30)alkylboronyl, (C1-C30)alkyl(C6-C30)arylboronyl, (C6-C30)ar(C1-C30)alkyl, and (C1-C30)alkyl(C6-C30)aryl. For example, the substituent may be at least one of deuterium, cyano, methyl, phenyl, biphenyl, naphthyl, phenanthrenyl, pyridyl unsubstituted or substituted with at least on of methyl and phenyl, or quinolinyl, etc.

In formulas of the present disclosure, when a plurality of substituents represented by the same symbol are present, each of the substituents represented by the same symbol may be the same or different.

The same reference numerals refer to the same elements herein. In addition, in the drawings, the thickness, ratio, and dimensions of the elements are only exaggerated for effective description of technical content, and this does not change the technical content.

Hereinafter, an organic electroluminescent device according to one embodiment will be described.

In one embodiment, an organic electroluminescent device comprising at least one deuterated compound is provided. Specifically, the organic electroluminescent device according to one embodiment comprises an anode; a cathode; at least one light-emitting layer positioned between the anode and the cathode; and at least one organic layer positioned between the light-emitting layer and the cathode, wherein the organic layer includes at least one deuterated compound, and the light-emitting layer includes a light-emitting compound including an iridium (Ir), platinum (Pt), or boron (B) compound, but does not include a delayed fluorescent compound According to one embodiment, the degree of deuteriumization of the deuterated compounds contained in the organic layer may be 30% to 100%, preferably 40% to 100%, more preferably 50% to 100%, even more preferably 55% to 100%, and most preferably 30% to 99%. When deuterated with a number equal to or higher than the lower limit, the bond dissociation energy according to deuteration increases, thereby increasing the stability of the compound. When such a compound is used in an organic electroluminescent device, improved lifespan characteristics may be exhibited.

According to one embodiment, the deuterated compounds contained in the organic layer may be a compound represented by the following Formula 1.

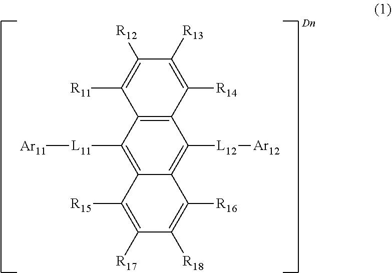

In Formula 1,

L₁₁ and L₁₂ each independently represent, a single bond, a substituted or unsubstituted (C6-C30)arylene, or a substituted or unsubstituted (3- to 30-membered)heteroarylene; Ar₁₁ and Ar₁₂ each independently represent, a substituted or unsubstituted (C6-C30)aryl or a substituted or unsubstituted (3- to 30-membered)heteroaryl; R₁₁ to R₁₈ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
at least one of R₁₁ and R₁₄ to R₁₆ is deuterium; and
Dn means that n number of hydrogens is replaced with deuterium, wherein the upper limit of n is determined by the number of hydrogens that can be substituted in each compound.

In one embodiment, L₁₁ and L₁₂ each independently may be a single bond or a substituted or unsubstituted (C6-C30)arylene, preferably a single bond or a substituted or unsubstituted (C6-C25)arylene, more preferably a single bond or a substituted or unsubstituted (C6-C18)arylene. For example, L₁₁ and L₁₂ each independently may be a single bond or phenylene.

In one embodiment, Ar₁₁ and Ar₁₂ each independently may be a substituted or unsubstituted (C6-C30)aryl or a substituted or unsubstituted (5- to 30-membered)heteroaryl, preferably, a substituted or unsubstituted (C6-C25)aryl or a substituted or unsubstituted (5 to 25-membered)heteroaryl, and more preferably, a substituted or unsubstituted (C6-C25)aryl or a substituted or unsubstituted (5- to 20-membered)heteroaryl. For example, Ar₁₁ and Ar₁₂ each independently may be a substituted or unsubstituted phenyl, a substituted or unsubstituted naphthyl, a substituted or unsubstituted m-biphenyl, a substituted or unsubstituted p-biphenyl, or a substituted or unsubstituted benzimidazolyl represented by the following Formula 1-1 or 1-2.

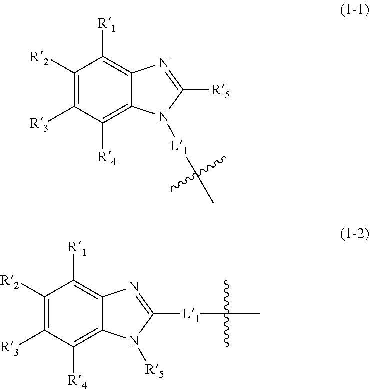

in Formulas 1-1 and 1-2,
L′₁ represents a single bond, a substituted or unsubstituted (C6-C30)arylene, or a substituted or unsubstituted (3- to 30-membered)heteroarylene; and
R′₁ to R′₅ each independently represent, hydrogen, deuterium, a substituted or unsubstituted (C1-C30)alkyl, a substituted or unsubstituted (C6-C30)aryl, or a substituted or unsubstituted (3- to 30-membered)heteroaryl.

In one embodiment, L′₁ may be a single bond or a substituted or unsubstituted (C6-C30)arylene, preferably a single bond or a substituted or unsubstituted (C6-C25)arylene, more preferably a single bond or a substituted or unsubstituted (C6-C18)arylene. For example, L′₁ may be a single bond or phenylene.

In one embodiment, R′₁ to R′₄ each independently may be hydrogen or deuterium.

In one embodiment, R′₅ may be a substituted or unsubstituted (C1-C30)alkyl or a substituted or unsubstituted (C6-C30)aryl, preferably a substituted or unsubstituted (C1-C10)alkyl or a substituted or unsubstituted (C6-C25)aryl, more preferably a substituted or unsubstituted (C1-C4)alkyl or a substituted or unsubstituted (C6-C18)aryl. For example, R′₅ may be ethyl, phenyl, naphthyl, or biphenyl.

In one embodiment, R₁₁ to R₁₈ each independently may be hydrogen, deuterium, or a substituted or unsubstituted benzimidazolyl represented by the Formula 1-1 or 1-2.

In one embodiment, at least one of R₁₁ to R₁₈, Ar₁₁, and Ar₁₂ may be a substituted or unsubstituted benzimidazolyl represented by the Formula 1-1 or 1-2.

According to another embodiment, the deuterated compounds contained in the organic layer may be a compound represented by the following Formula 2.

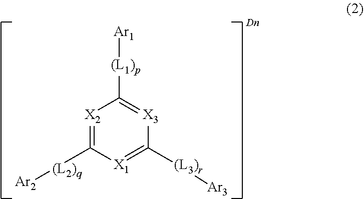

in Formula 2,
X₁ to X₃ each independently represent, CR′ or N; provided that at least two of X₁ to X₃ are N;
R′ represents hydrogen or deuterium; L₁ to L₃ each independently represent, a single bond, a substituted or unsubstituted (C6-C30)arylene, or a substituted or unsubstituted (3- to 30-membered)heteroarylene;
Ar₁ to Ar₃ each independently represent, a substituted or unsubstituted (C6-C30)aryl or a substituted or unsubstituted (3- to 30-membered)heteroaryl; provided that at least one of Ar₁ toAr₃ includes deuterium;
p, q, and r each independently represent, an integer of 1 to 3, when p, q, and r are 2 or more, each of L₁ to L₃ may be the same or different; and
Dn means that n of the hydrogens are replaced with deuterium, wherein the upper limit of n is determined according to the number of hydrogens that may be substituted for each compound.

In one embodiment, at least two of X₁ to X₃ may be N, preferably all of X₁ to X₃ may be N.

In one embodiment, L₁ to L₃ each independently may be a single bond or a substituted or unsubstituted (C6-C30)arylene, preferably L₁ to L₃ each independently may be a single bond or a substituted or unsubstituted (C6-C25)arylene, more preferably a single bond or a substituted or unsubstituted (C6-C18)arylene. For example, L₁ to L₃ each independently may be a single bond, a substituted or unsubstituted phenylene, a substituted or unsubstituted naphthylene, a substituted or unsubstituted p-biphenylene, a substituted or unsubstituted m-biphenylene, or a substituted or unsubstituted o-terphenylene. Wherein, the substituents of the substituted groups may be deuterium, phenanthrenyl, pyridyl unsubstituted or unsubstituted with at least one of methyl or phenyl, or quinolinyl.

In one embodiment, Ar₁ to Ar₃ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 26-membered)heteroaryl, more preferably a substituted or unsubstituted (C6-C18)aryl or a substituted or unsubstituted (5- to 26-membered)heteroaryl. For example, Arₙ to Ar₃ each independently may be a substituted or unsubstituted phenyl, a substituted or unsubstituted naphthyl, a substituted or unsubstituted p-biphenyl, a substituted or unsubstituted m-biphenyl, a substituted or unsubstituted phenanthrenyl, a substituted or unsubstituted fluorenyl, a substituted or unsubstituted pyridyl, a substituted or unsubstituted triazinyl, a substituted or unsubstituted quinolinyl, a substituted or unsubstituted spiro[fluorene-9,9′-xanthene]yl, or 22-membered heteroaryl. Wherein, the substituents of the substituted groups may be deuterium, cyano, methyl, phenyl, biphenyl, or naphthyl.

According to one embodiment, the deuterated compound may be more specifically illustrated by the following compounds, but is not limited thereto.

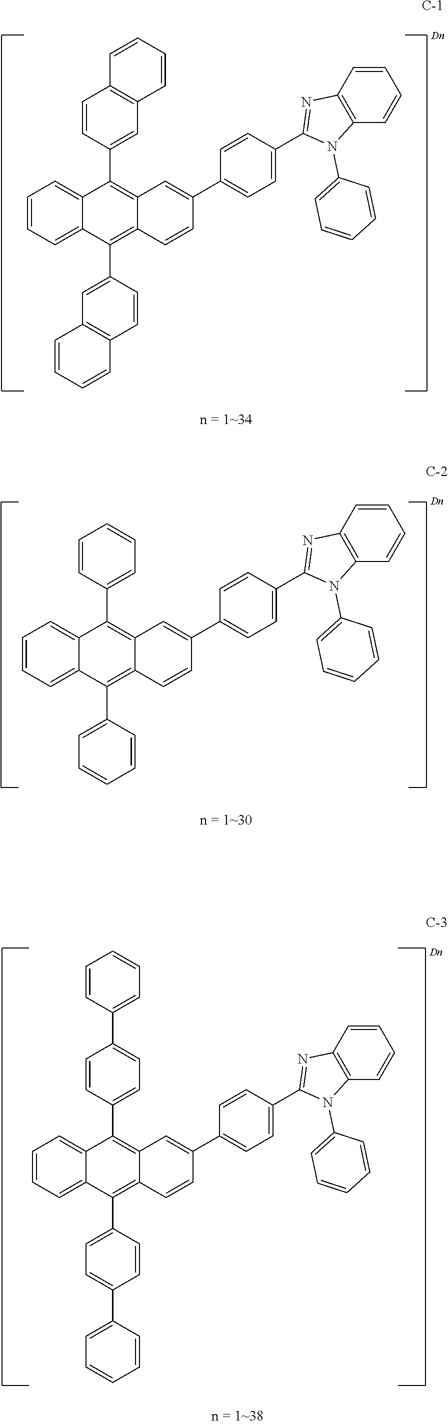

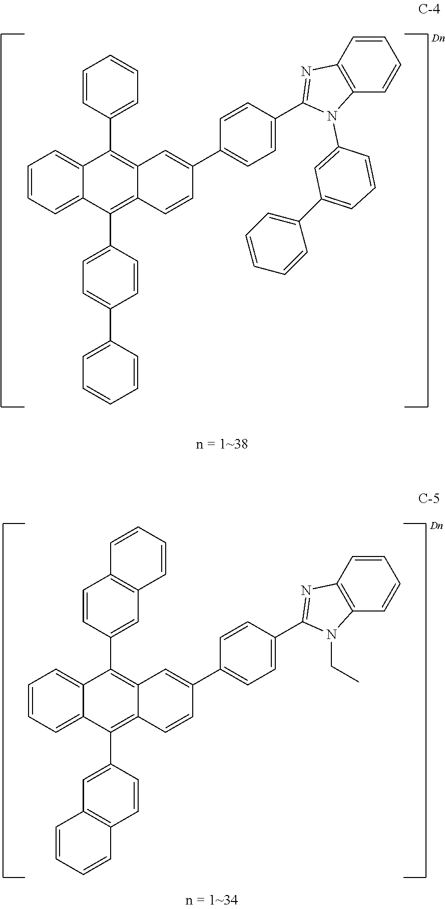

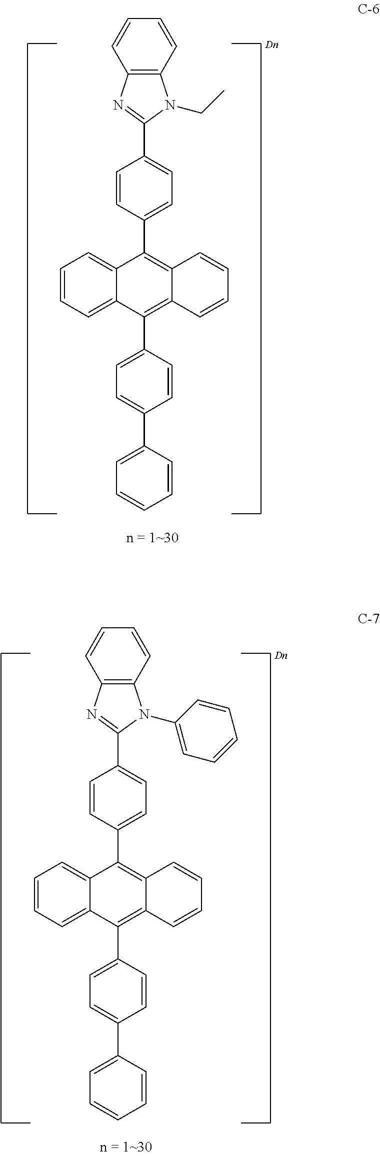

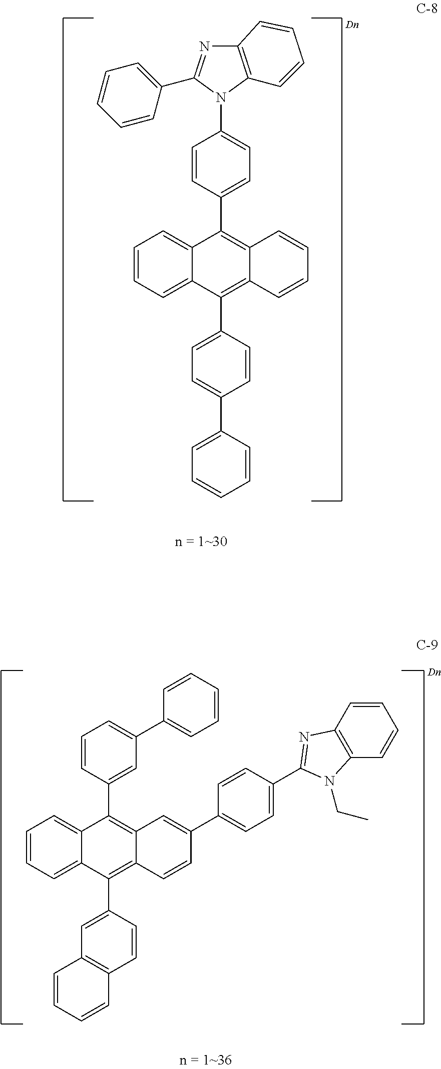

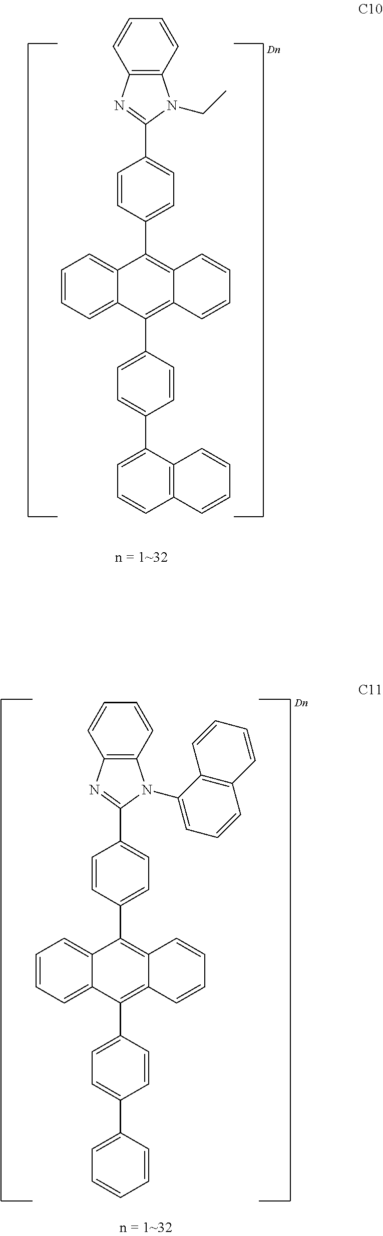

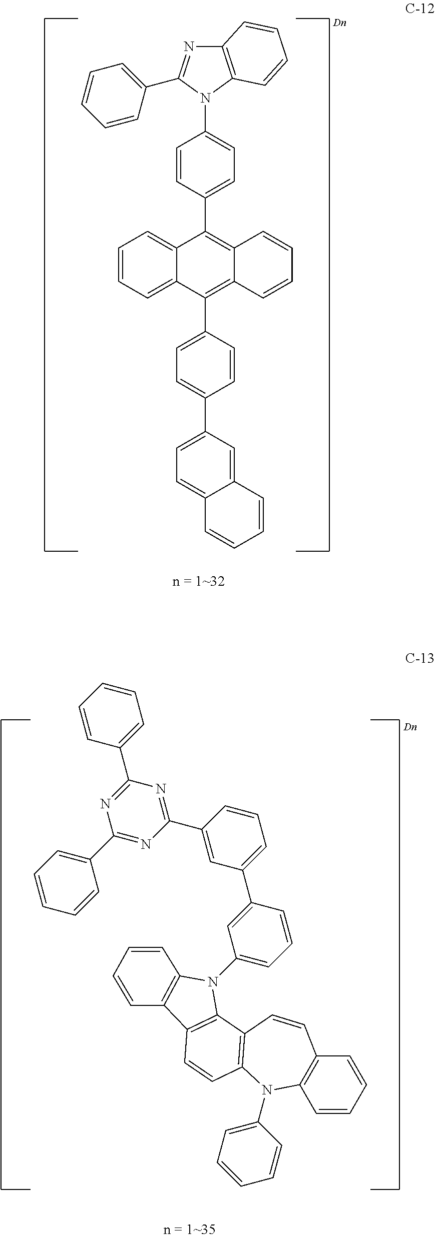

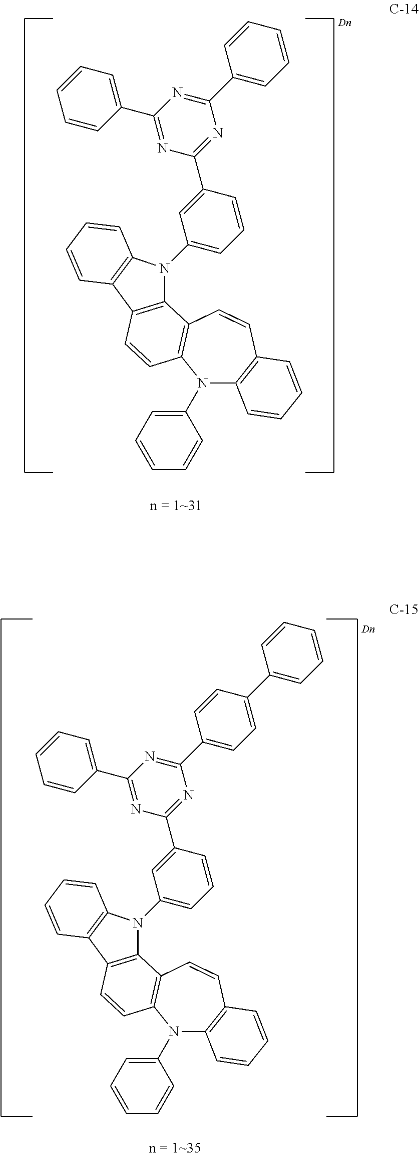

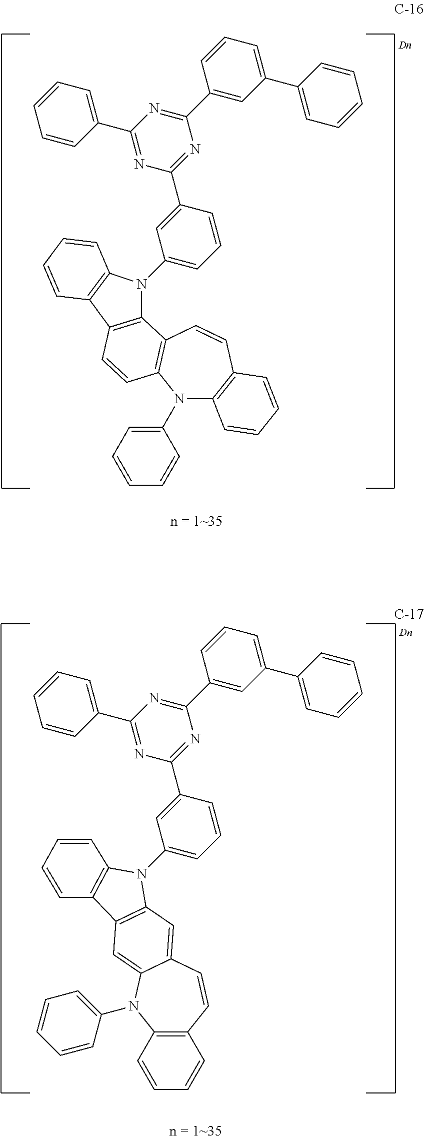

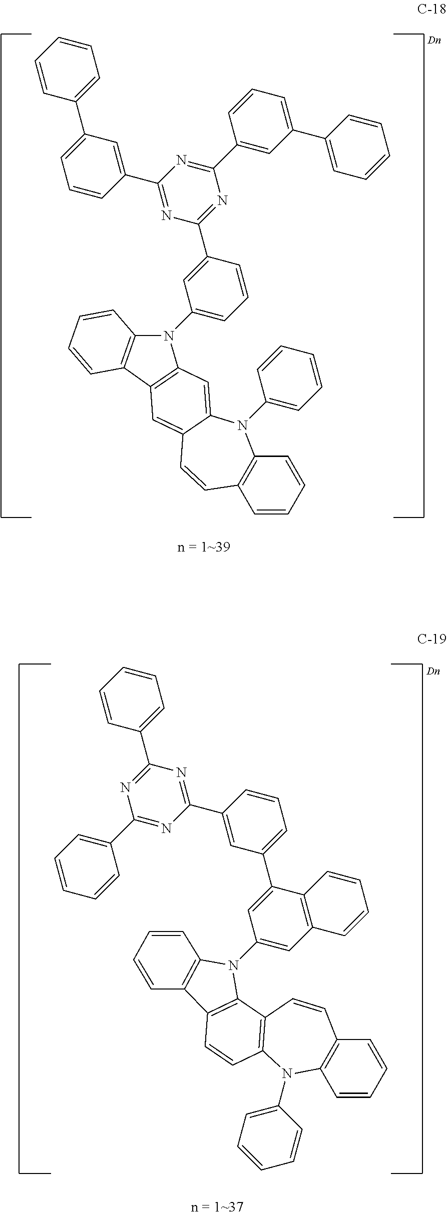

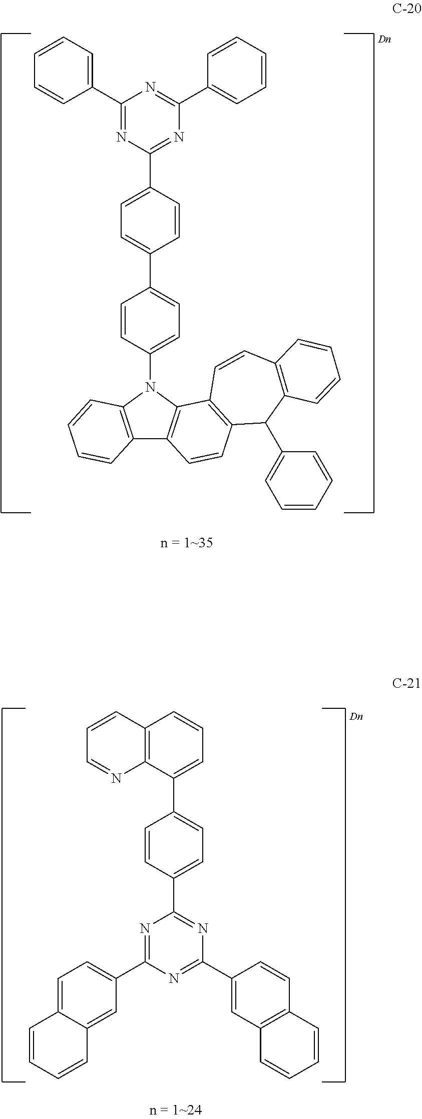

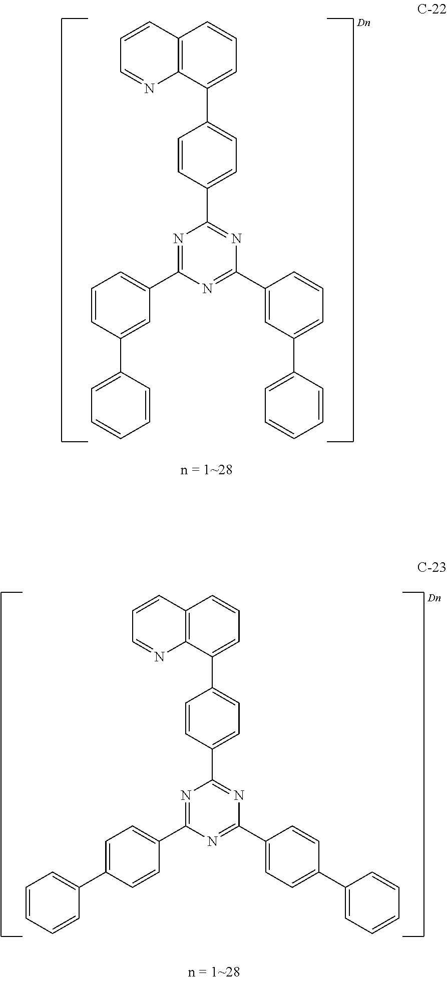

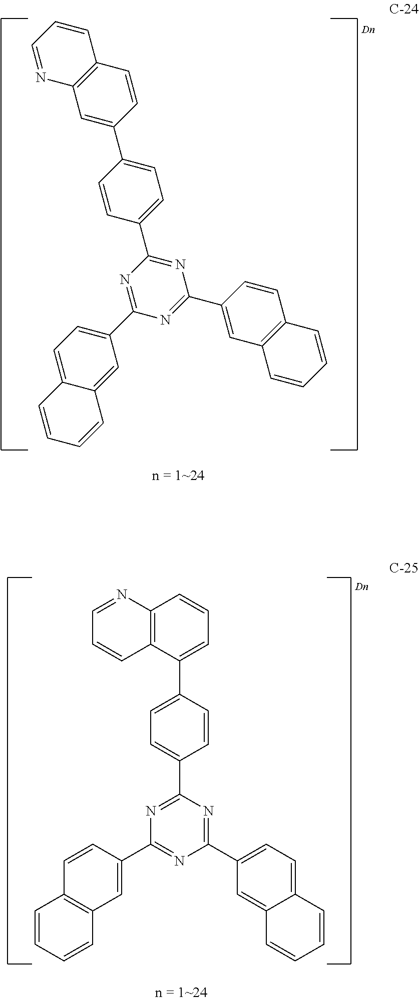

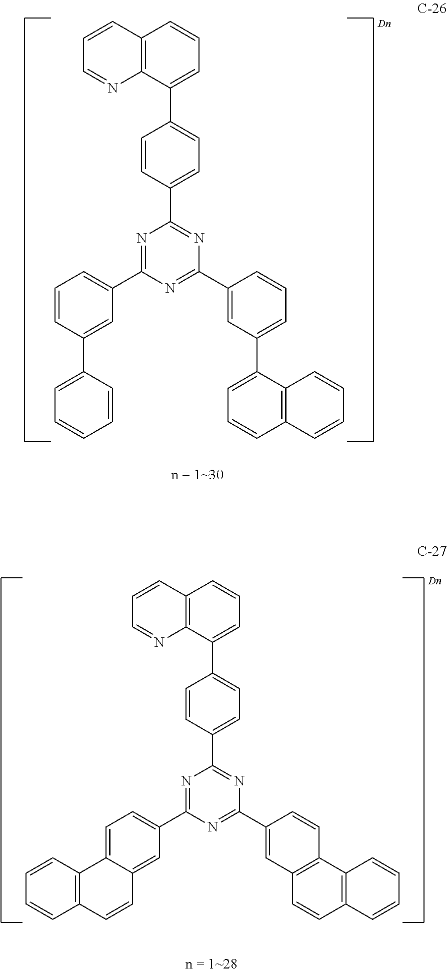

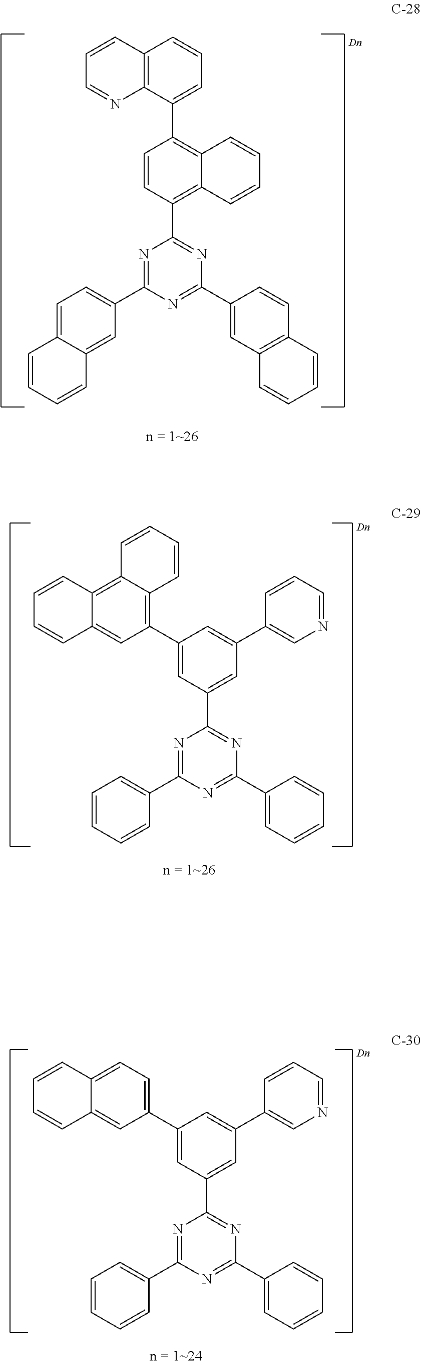

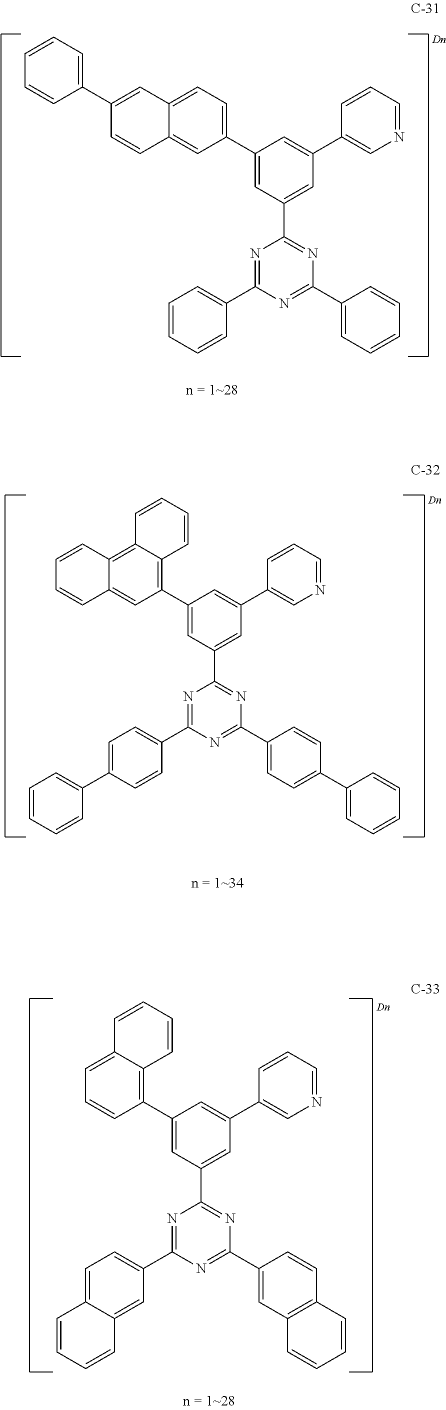

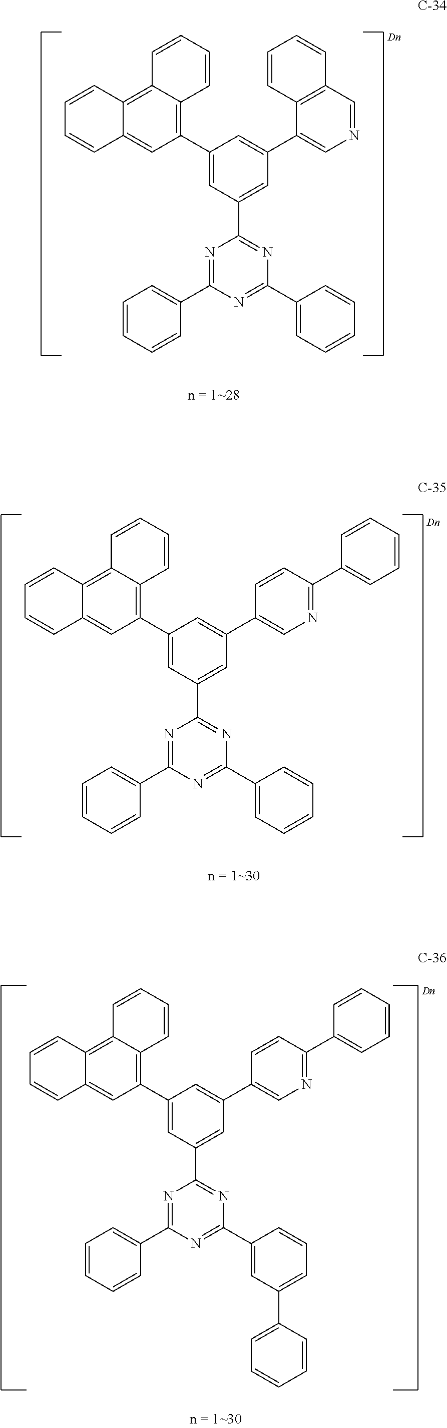

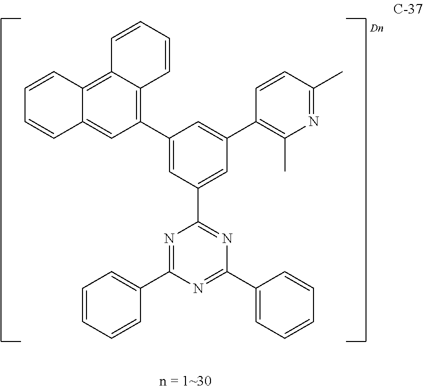

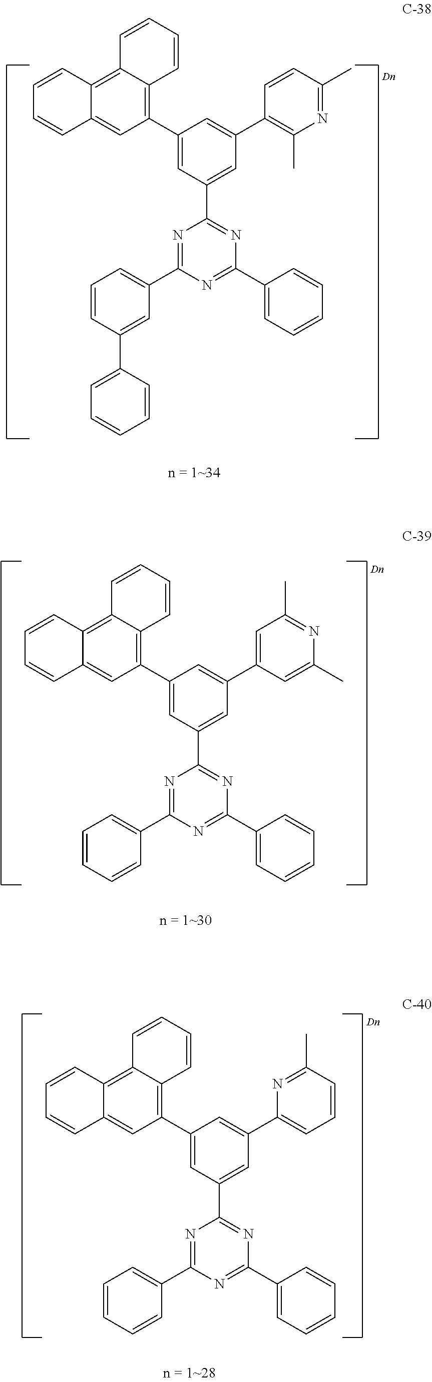

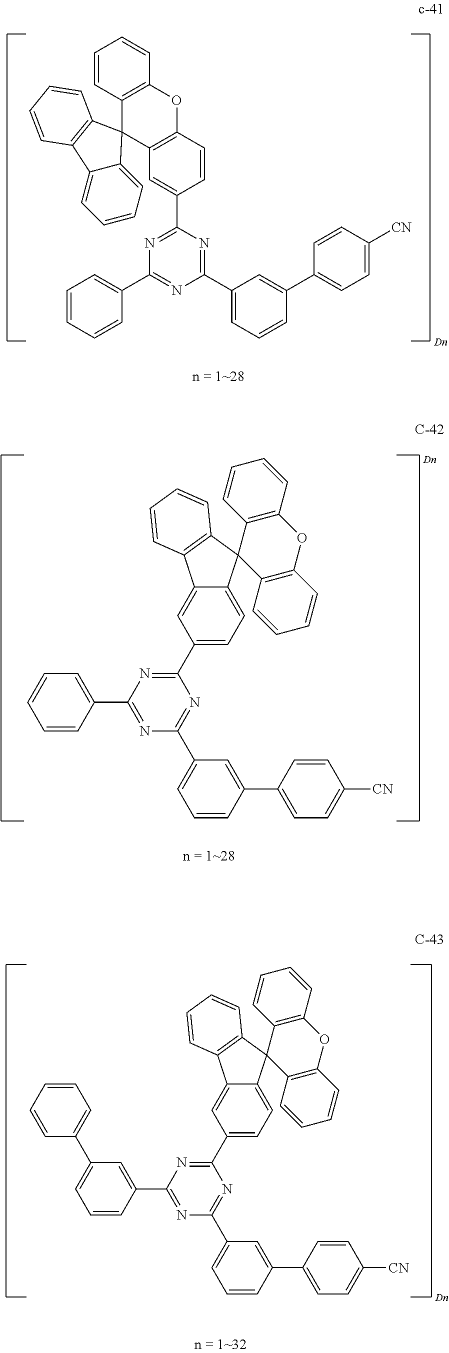

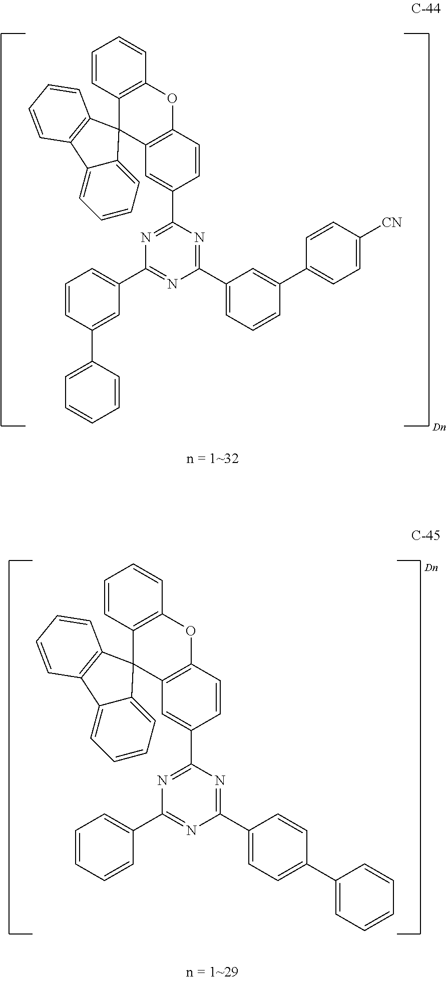

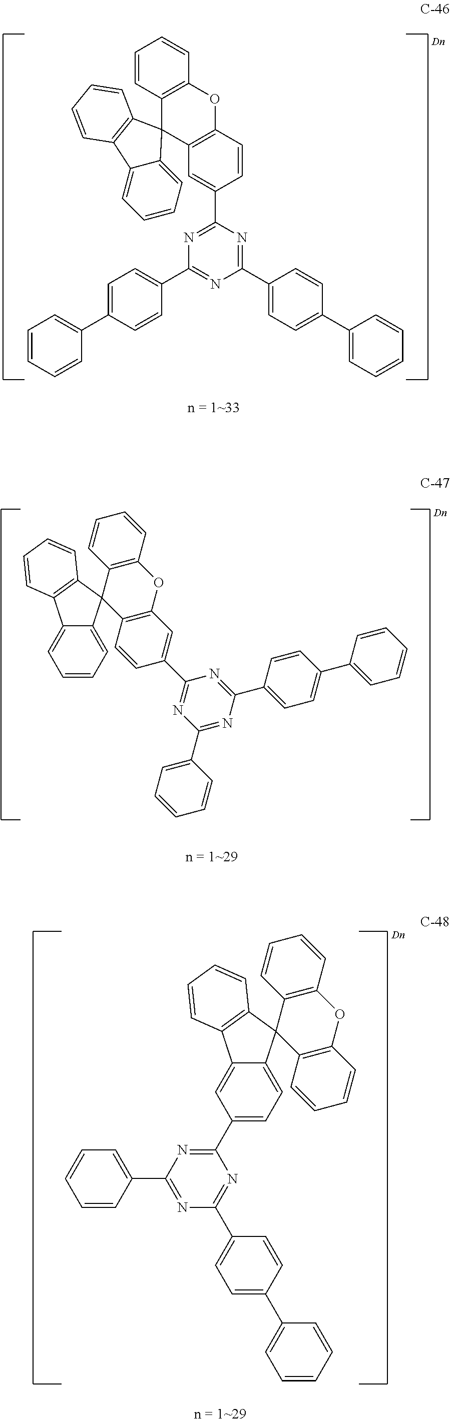

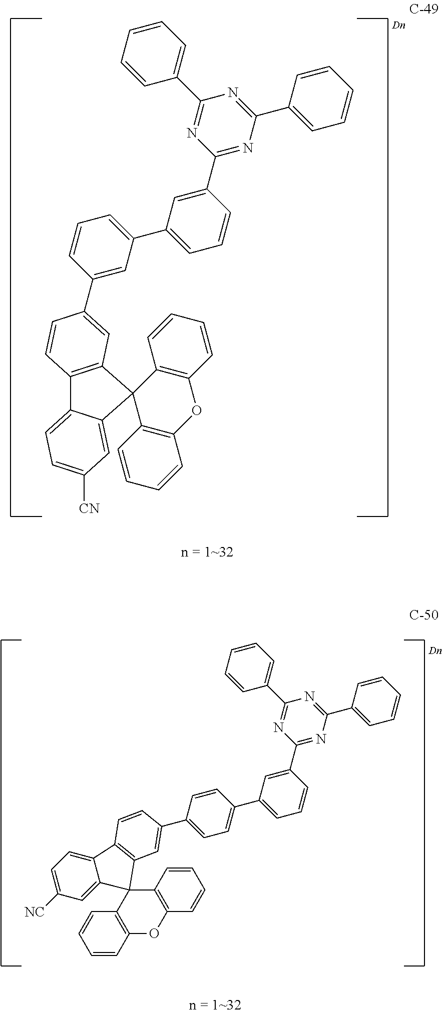

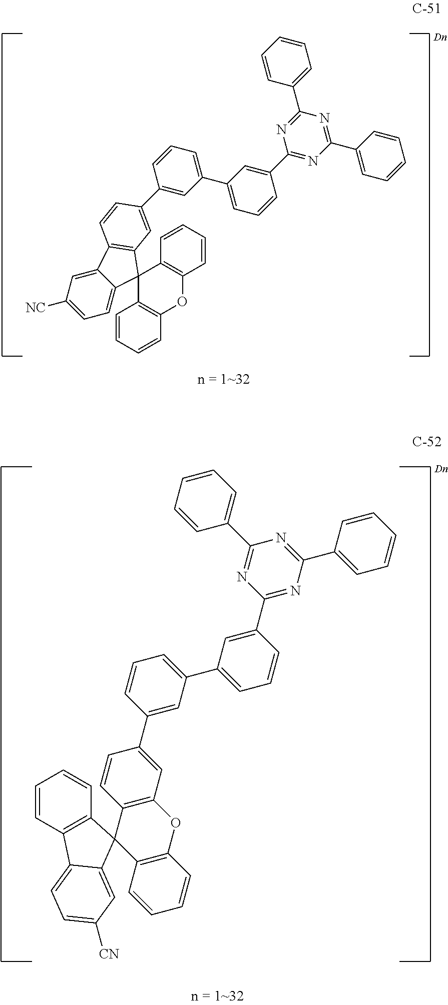

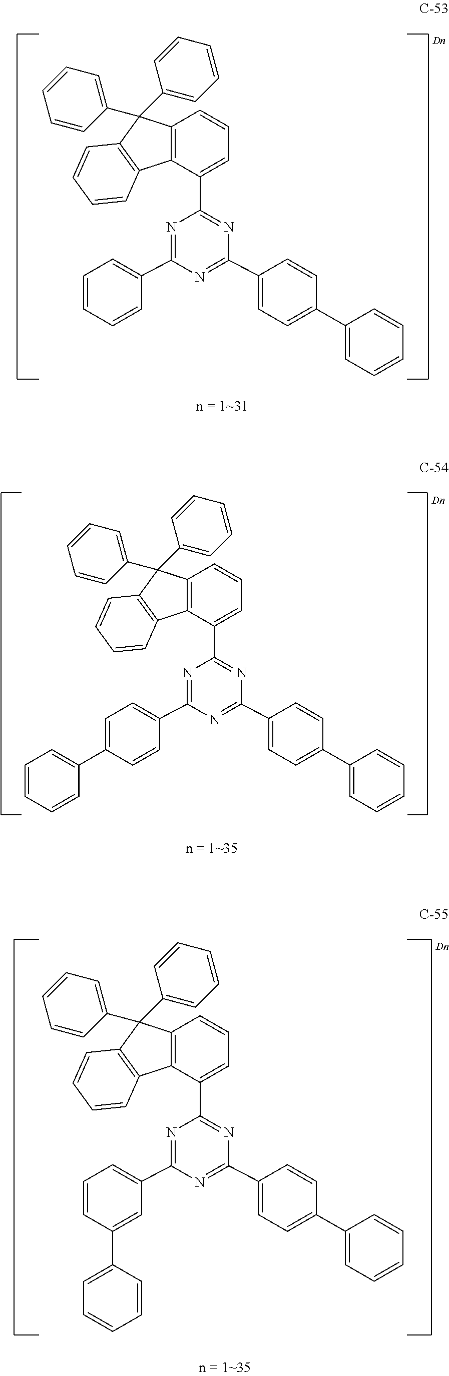

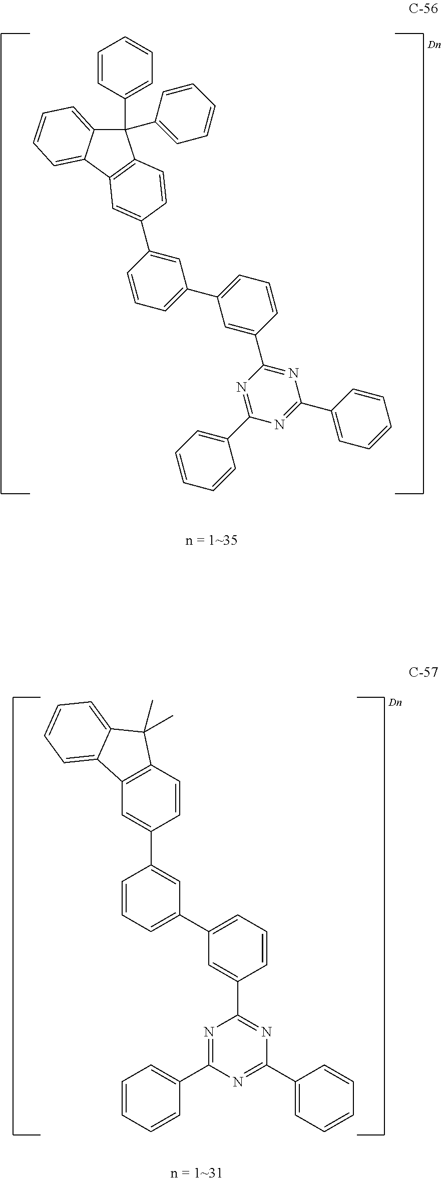

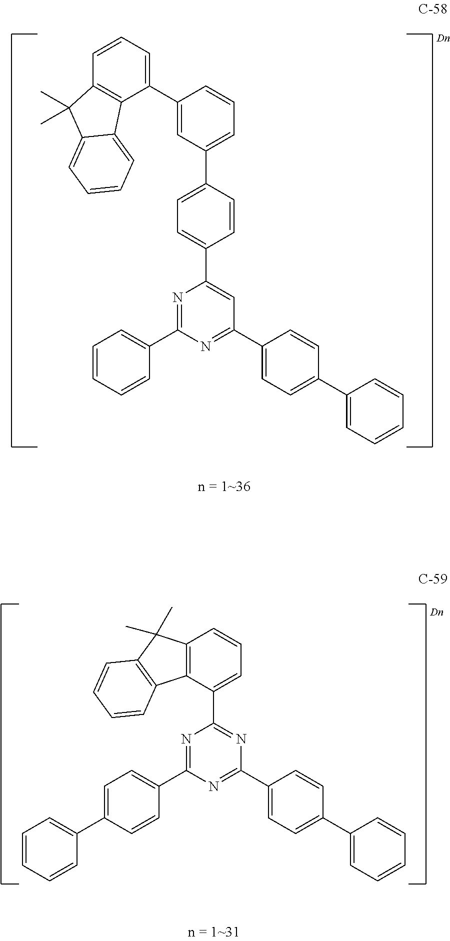

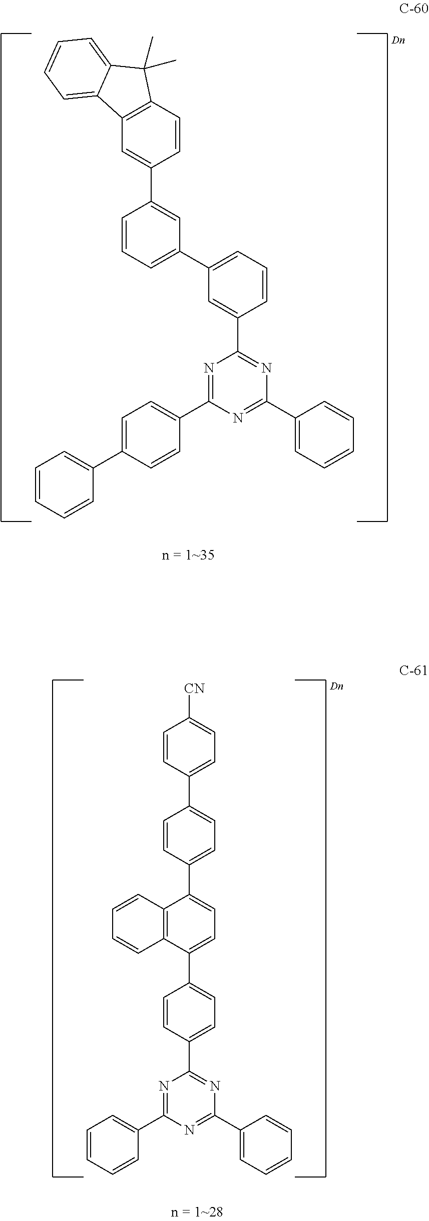

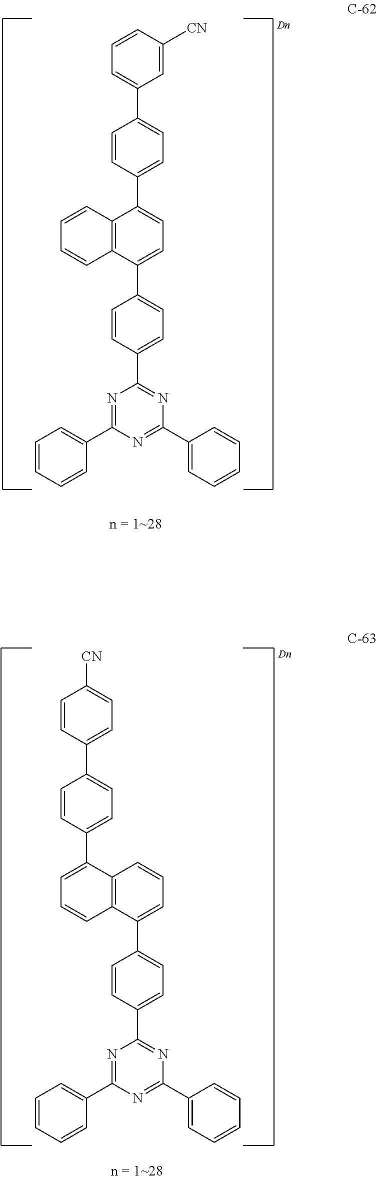

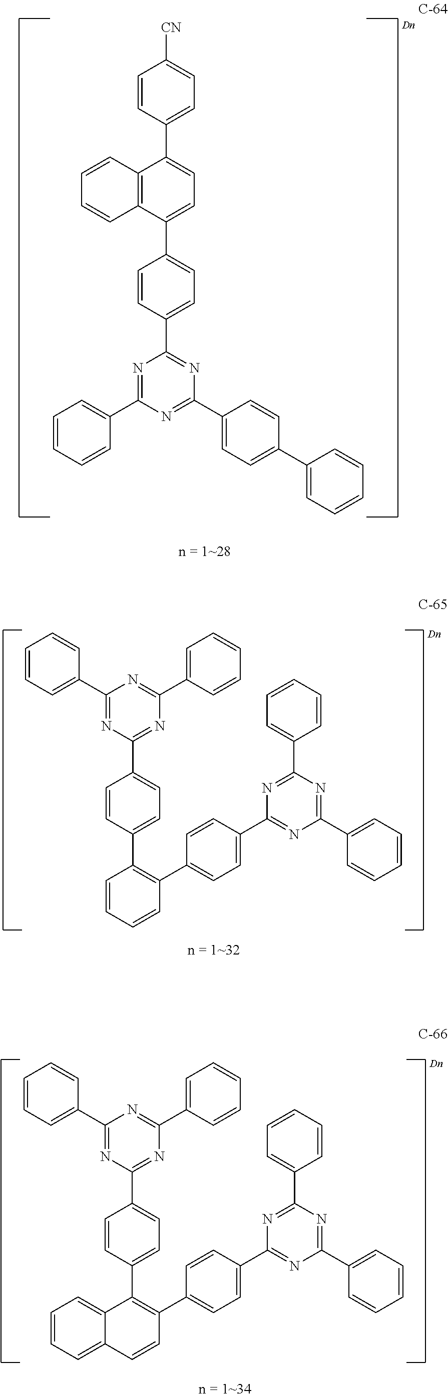

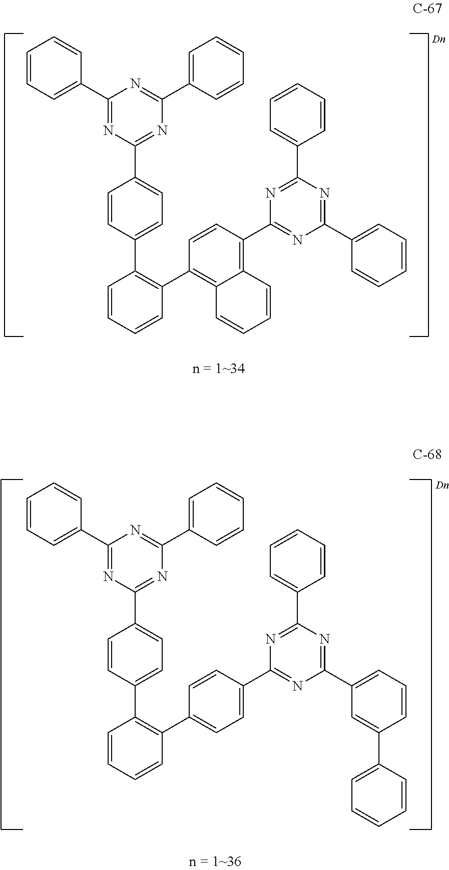

In the compounds above, Dn means that n number of hydrogens is replaced with deuterium, wherein the upper limit of n is determined by the number of hydrogens that can be substituted in each compound.

The deuterated compound represented by Formula 1 or 2 according to the present disclosure can be prepared by a known synthetic method, but is not limited thereto. The deuterated compound of Formula 1 or 2 can be prepared using a deuterated precursor material in a similar manner, or more generally can be prepared by treating a non-deuterated compound with a deuterated solvent, D6-benzene in the presence of a Lewis acid H/D exchange catalyst such as aluminum trichloride or ethyl aluminum chloride. In addition, the degree of deuterization can be controlled by varying reaction conditions such as reaction temperature. For example, the number of deuterium in Formula 1 or 2 can be adjusted by controlling the reaction temperature and time, the equivalent of acid, etc.

The light-emitting layer according to one embodiment includes a light-emitting compound comprising iridium (Ir), platinum (Pt), or boron (B) compound. However, the light-emitting layer does not include a delayed fluorescence compound.

The light-emitting layer according to one embodiment may comprise a light-emitting compound represented by the following Formula 11.

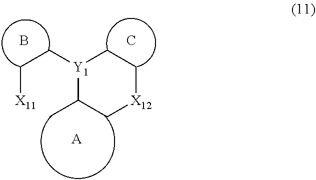

in Formula 11,
Ring A, Ring B, and Ring C each independently represent, a substituted or unsubstituted (C6-C30)aryl or a substituted or unsubstituted (3- to 50-membered)heteroaryl;
Y₁ represents B;
X₁₁ and X₁₂ each independently represent, NRₐ, O or S;
Rₐ represents hydrogen, deuterium, halogen, cyano, a substituted or unsubstituted (C1-C30)alkyl, a substituted or unsubstituted (C6-C30)aryl, a substituted or unsubstituted (3 to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N—(Ar₄)(Ar₅); or Rₐ may be linked to at least one of Ring A, Ring B, and Ring C to form a ring(s);
L₄ represents a single bond, a substituted or unsubstituted (C6-C30)aryl, a substituted or unsubstituted (3- to 30-membered)heteroaryl, or a substituted or unsubstituted divalent (C2-C30) aliphatic hydrocarbon group, or a substituted or unsubstituted fused ring of divalent (C3-C30) aliphatic ring and (C6-C30) aromatic ring; and
Ar₄ and Ar₅ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

According to one embodiment, the light-emitting compound may be more specifically illustrated by the following compounds, but is not limited thereto.

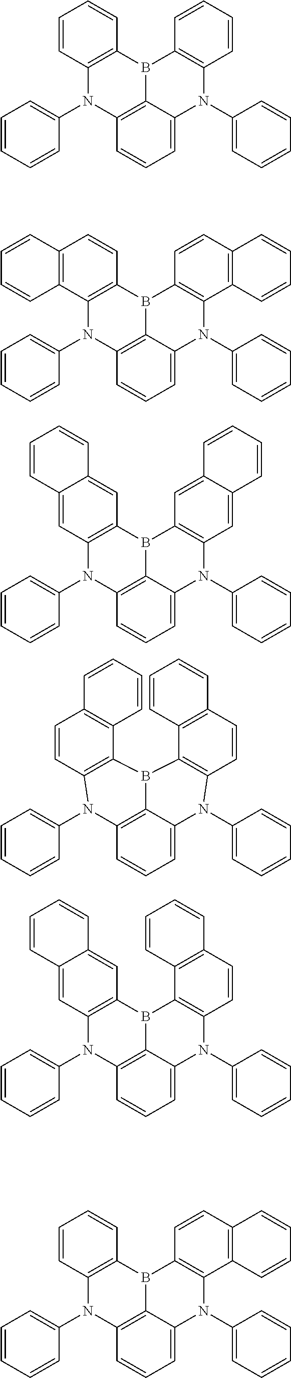

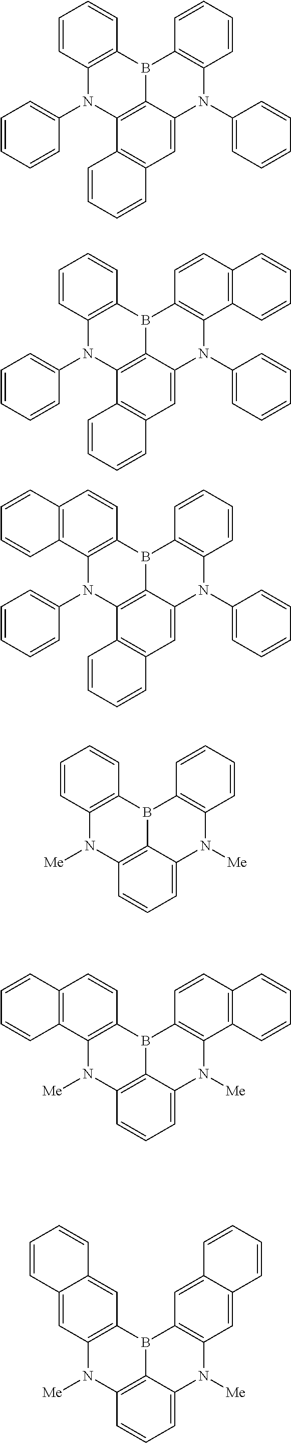

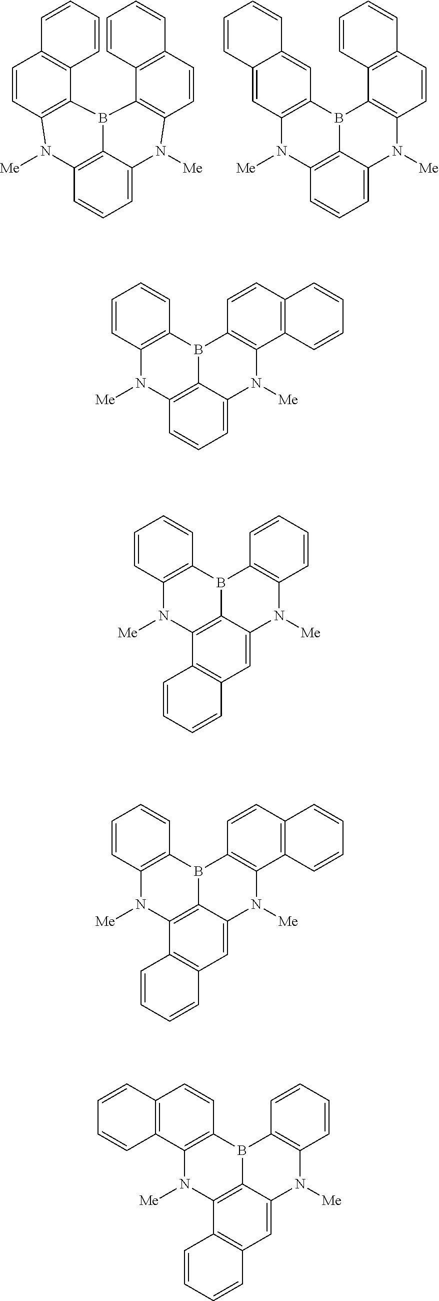

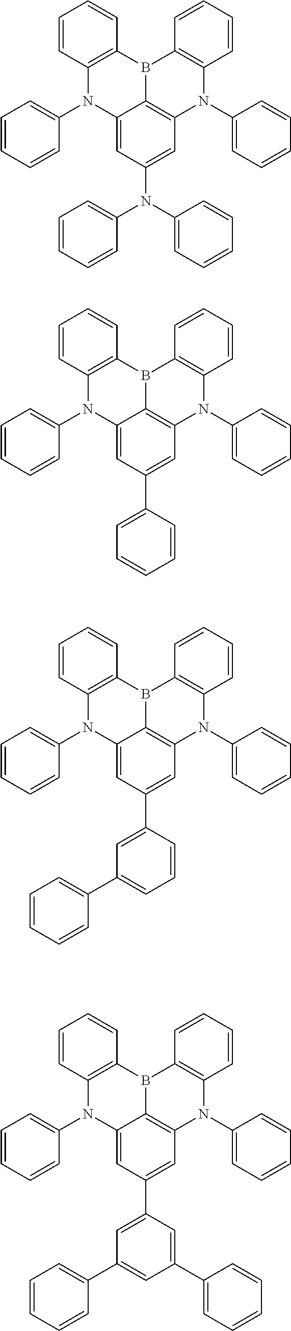

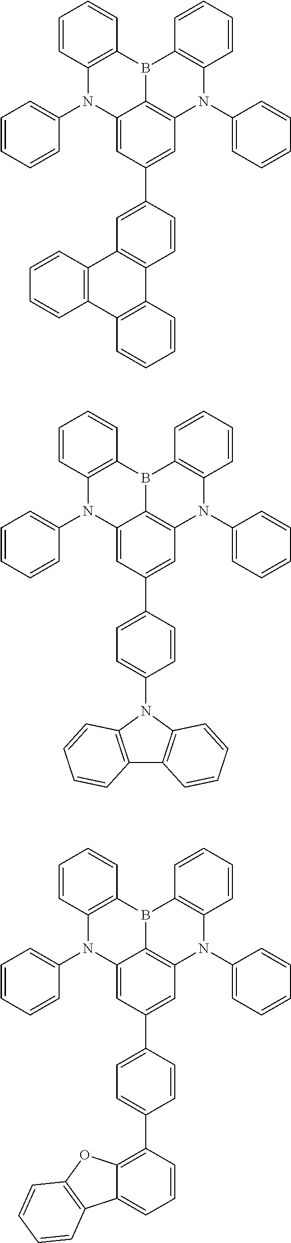 [SMILES: c1ccc(N2c3ccccc3B3c4ccccc4N(c4ccccc4)c4cc(-c5ccc6c7ccccc7c7ccccc7c6c5)cc2c43)cc1 ; c1ccc(N2c3ccccc3B3c4ccccc4N(c4ccccc4)c4cc(-c5ccc(-n6c7ccccc7c7ccccc76)cc5)cc2c43)cc1 ; c1ccc(N2c3ccccc3B3c4ccccc4N(c4ccccc4)c4cc(-c5ccc(-c6cccc7c6oc6ccccc67)cc5)cc2c43)cc1]

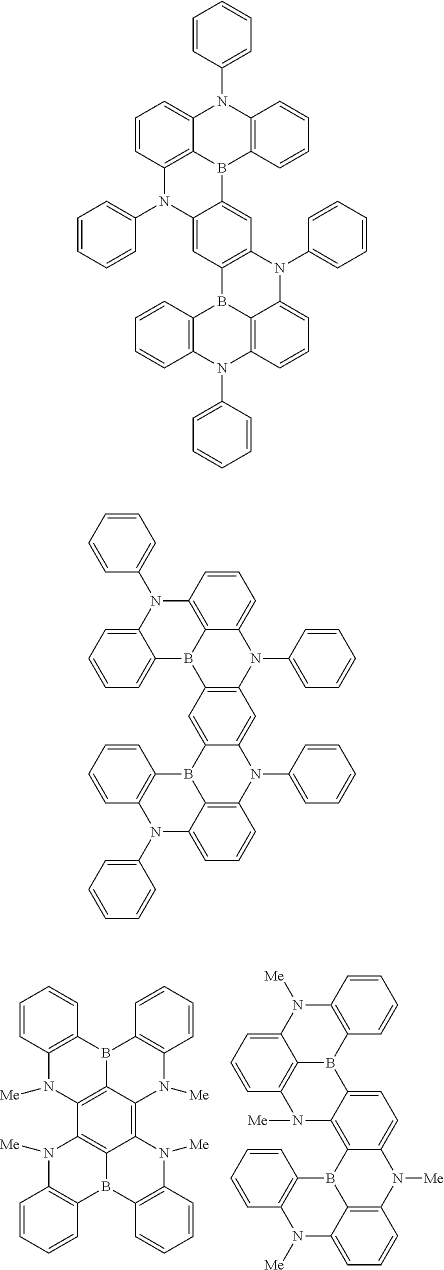

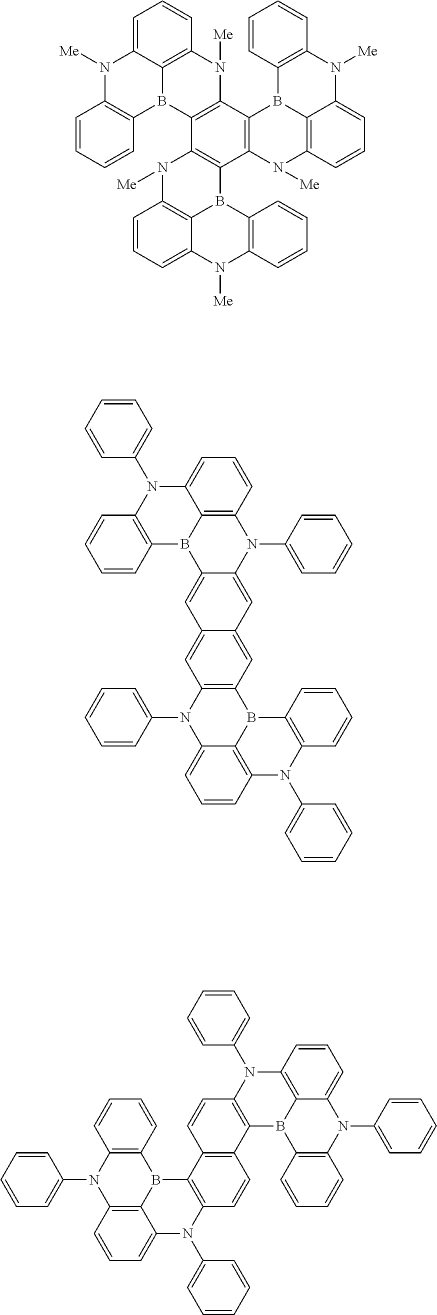 [SMILES: CN1c2ccccc2B2c3c1cccc3N(C)c1c3c(c4c(c12)N(C)c1cccc2c1B4c1ccccc1N2C)N(C)c1cccc2c1B3c1ccccc1N2C ; c1ccc(N2c3ccccc3B3c4cc5cc6c(cc5cc4N(c4ccccc4)c4cccc2c43)B2c3ccccc3N(c3ccccc3)c3cccc(c32)N6c2ccccc2)cc1 ; c1ccc(N2c3ccccc3B3c4c2cccc4N(c2ccccc2)c2ccc4c5c(ccc4c23)N(c2ccccc2)c2cccc3c2B5c2ccccc2N3c2ccccc2)cc1]

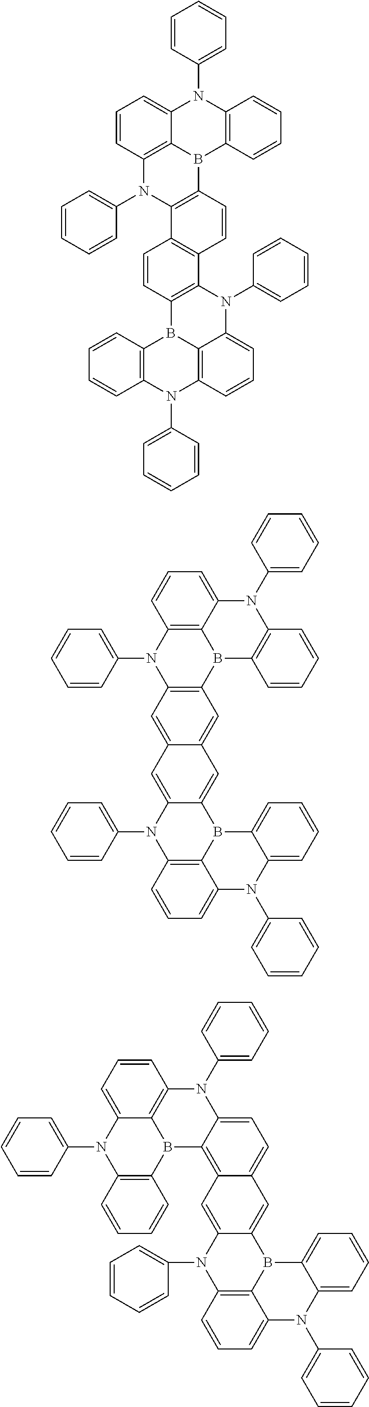

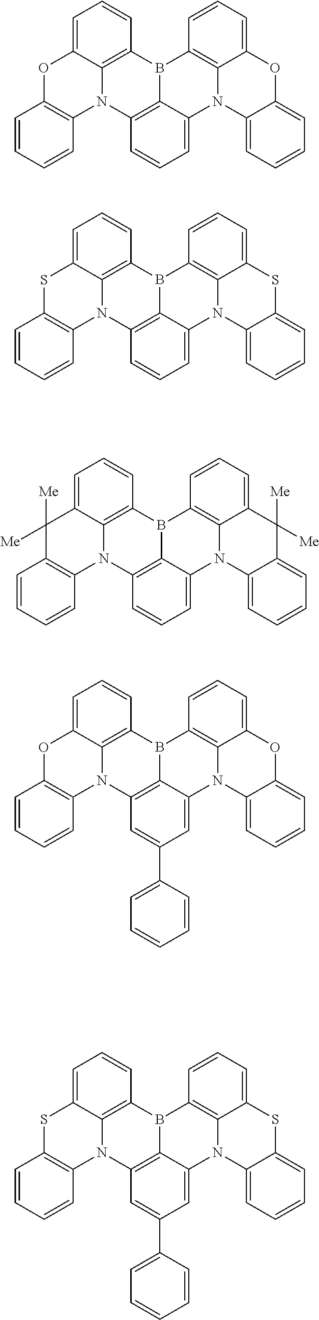 [SMILES: c1ccc2c(c1)Oc1cccc3c1N2c1cccc2c1B3c1cccc3c1N2c1ccccc1O3 ; c1ccc2c(c1)Sc1cccc3c1N2c1cccc2c1B3c1cccc3c1N2c1ccccc1S3 ; CC1(C)c2ccccc2N2c3cccc4c3B(c3cccc1c32)c1cccc2c1N4c1ccccc1C2(C)C ; c1ccc(-c2cc3c4c(c2)N2c5ccccc5Oc5cccc(c52)B4c2cccc4c2N3c2ccccc2O4)cc1 ; c1ccc(-c2cc3c4c(c2)N2c5ccccc5Sc5cccc(c52)B4c2cccc4c2N3c2ccccc2S4)cc1]

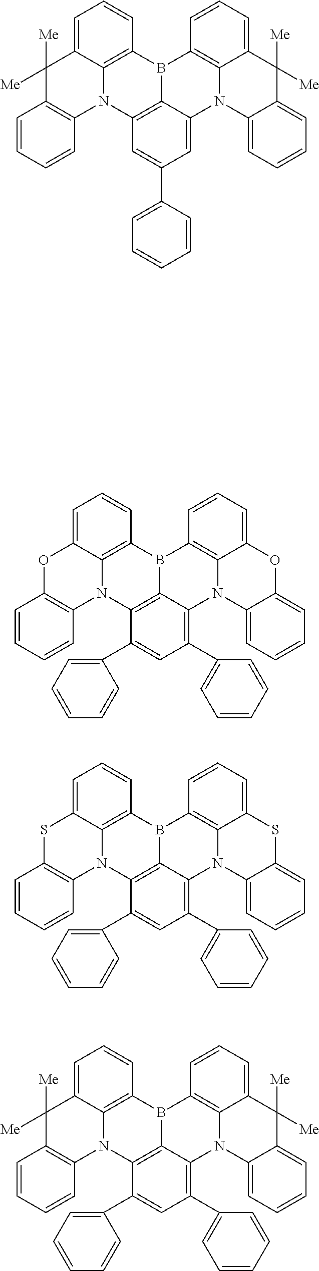

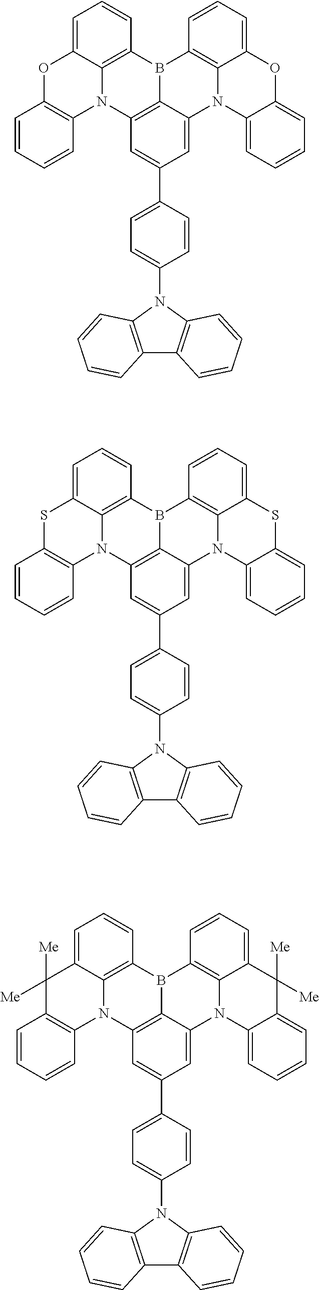 [SMILES: c1ccc2c(c1)Oc1cccc3c1N2c1cc(-c2ccc(-n4c5ccccc5c5ccccc54)cc2)cc2c1B3c1cccc3c1N2c1ccccc1O3 ; c1ccc2c(c1)Sc1cccc3c1N2c1cc(-c2ccc(-n4c5ccccc5c5ccccc54)cc2)cc2c1B3c1cccc3c1N2c1ccccc1S3 ; CC1(C)c2ccccc2N2c3cc(-c4ccc(-n5c6ccccc6c6ccccc65)cc4)cc4c3B(c3cccc1c32)c1cccc2c1N4c1ccccc1C2(C)C]

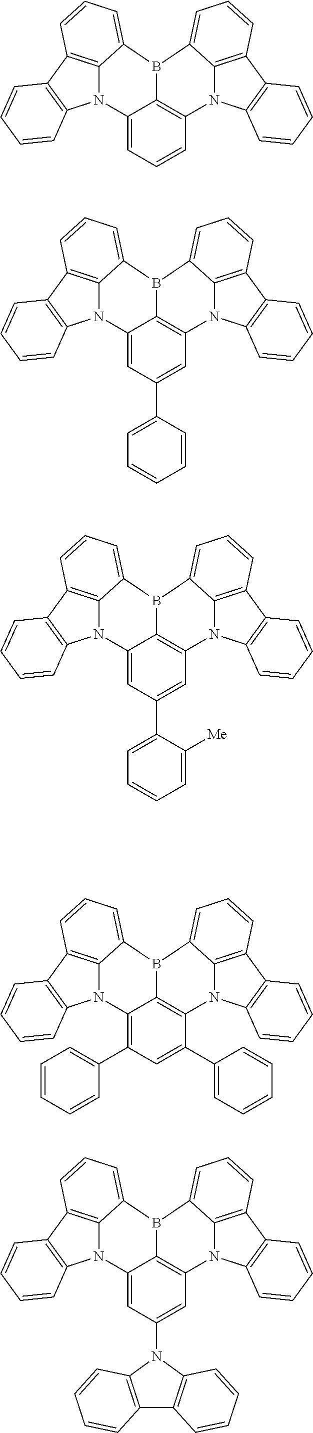

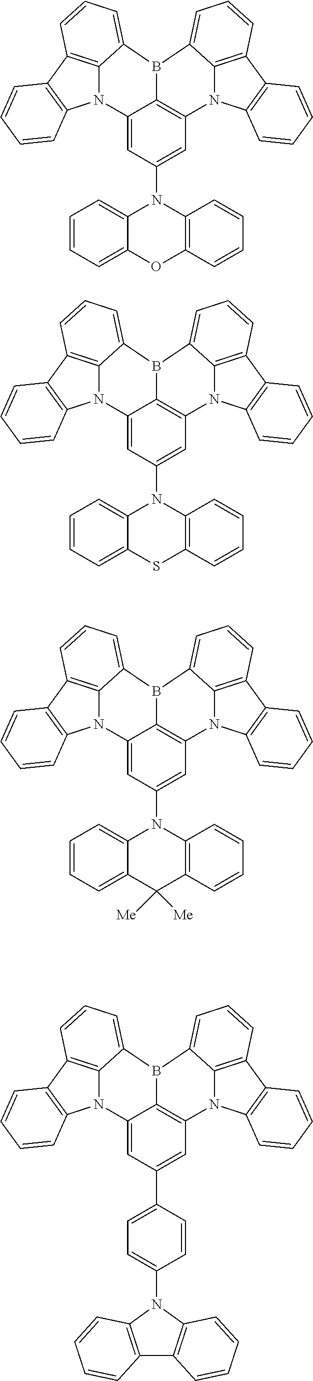

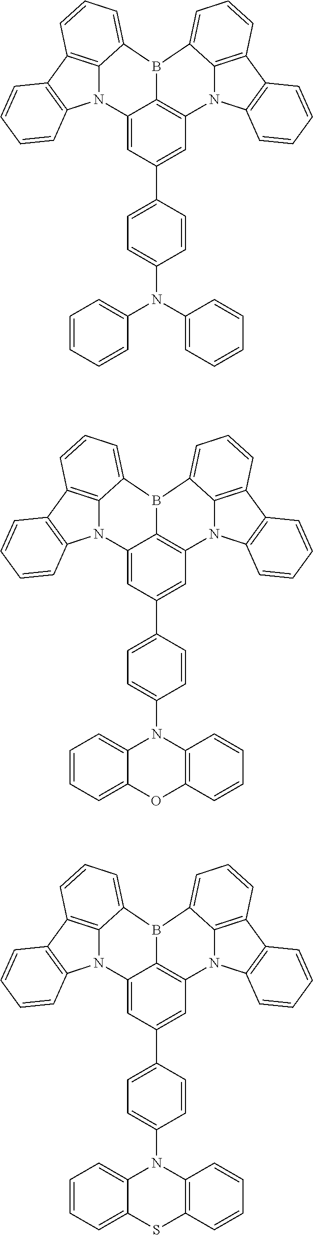 [SMILES: c1ccc(N(c2ccccc2)c2ccc(-c3cc4c5c(c3)-n3c6ccccc6c6cccc(c63)B5c3cccc5c6ccccc6n-4c35)cc2)cc1 ; c1ccc2c(c1)Oc1ccccc1N2c1ccc(-c2cc3c4c(c2)-n2c5ccccc5c5cccc(c52)B4c2cccc4c5ccccc5n-3c24)cc1 ; c1ccc2c(c1)Sc1ccccc1N2c1ccc(-c2cc3c4c(c2)-n2c5ccccc5c5cccc(c52)B4c2cccc4c5ccccc5n-3c24)cc1]

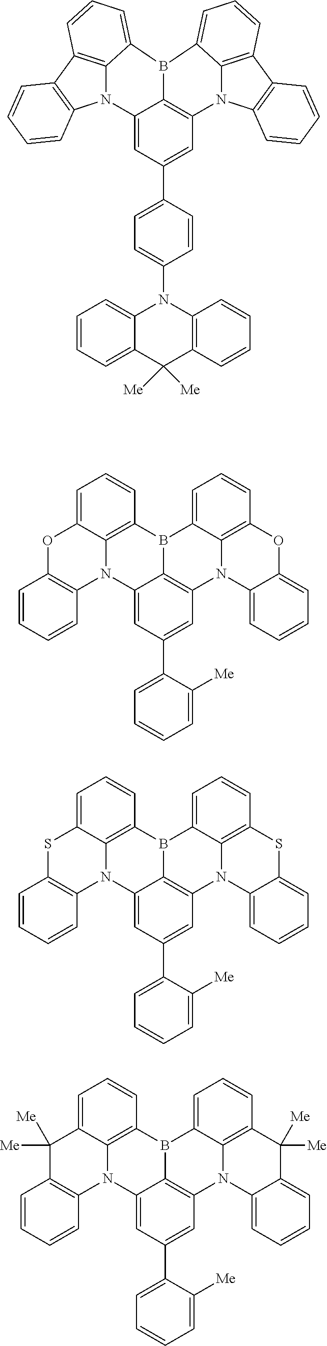 [SMILES: CC1(C)c2ccccc2N(c2ccc(-c3cc4c5c(c3)-n3c6ccccc6c6cccc(c63)B5c3cccc5c6ccccc6n-4c35)cc2)c2ccccc21 ; Cc1ccccc1-c1cc2c3c(c1)N1c4ccccc4Oc4cccc(c41)B3c1cccc3c1N2c1ccccc1O3 ; Cc1ccccc1-c1cc2c3c(c1)N1c4ccccc4Sc4cccc(c41)B3c1cccc3c1N2c1ccccc1S3 ; Cc1ccccc1-c1cc2c3c(c1)N1c4ccccc4C(C)(C)c4cccc(c41)B3c1cccc3c1N2c1ccccc1C3(C)C]

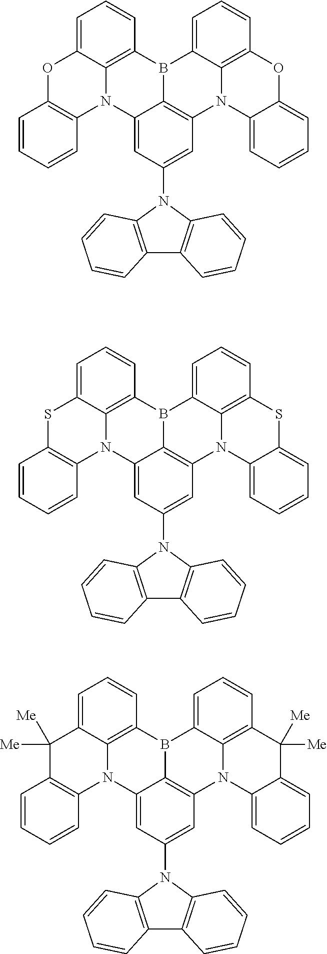 [SMILES: c1ccc2c(c1)Oc1cccc3c1N2c1cc(-n2c4ccccc4c4ccccc42)cc2c1B3c1cccc3c1N2c1ccccc1O3 ; c1ccc2c(c1)Sc1cccc3c1N2c1cc(-n2c4ccccc4c4ccccc42)cc2c1B3c1cccc3c1N2c1ccccc1S3 ; CC1(C)c2ccccc2N2c3cc(-n4c5ccccc5c5ccccc54)cc4c3B(c3cccc1c32)c1cccc2c1N4c1ccccc1C2(C)C]

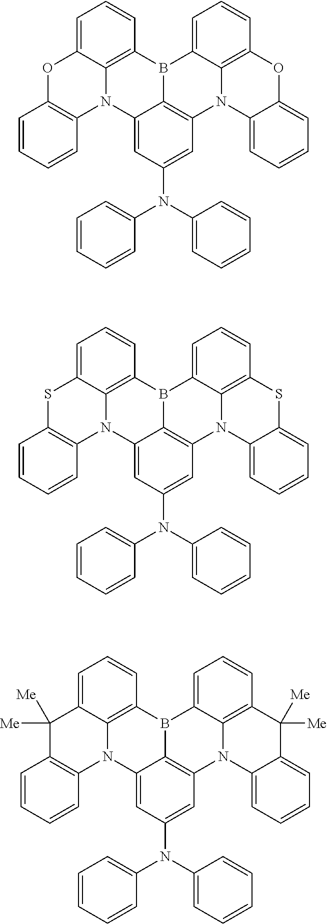 [SMILES: c1ccc(N(c2ccccc2)c2cc3c4c(c2)N2c5ccccc5Oc5cccc(c52)B4c2cccc4c2N3c2ccccc2O4)cc1 ; c1ccc(N(c2ccccc2)c2cc3c4c(c2)N2c5ccccc5Sc5cccc(c52)B4c2cccc4c2N3c2ccccc2S4)cc1 ; CC1(C)c2ccccc2N2c3cc(N(c4ccccc4)c4ccccc4)cc4c3B(c3cccc1c32)c1cccc2c1N4c1ccccc1C2(C)C]

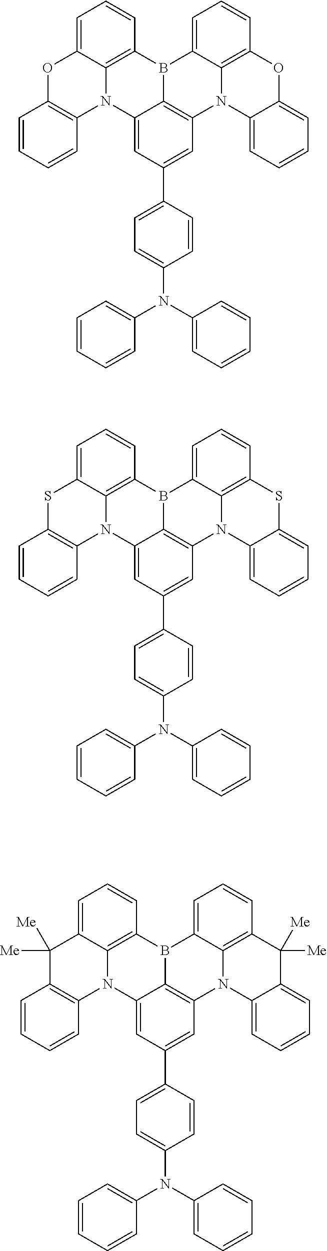 [SMILES: c1ccc(N(c2ccccc2)c2ccc(-c3cc4c5c(c3)N3c6ccccc6Oc6cccc(c63)B5c3cccc5c3N4c3ccccc3O5)cc2)cc1 ; c1ccc(N(c2ccccc2)c2ccc(-c3cc4c5c(c3)N3c6ccccc6Sc6cccc(c63)B5c3cccc5c3N4c3ccccc3S5)cc2)cc1 ; CC1(C)c2ccccc2N2c3cc(-c4ccc(N(c5ccccc5)c5ccccc5)cc4)cc4c3B(c3cccc1c32)c1cccc2c1N4c1ccccc1C2(C)C]

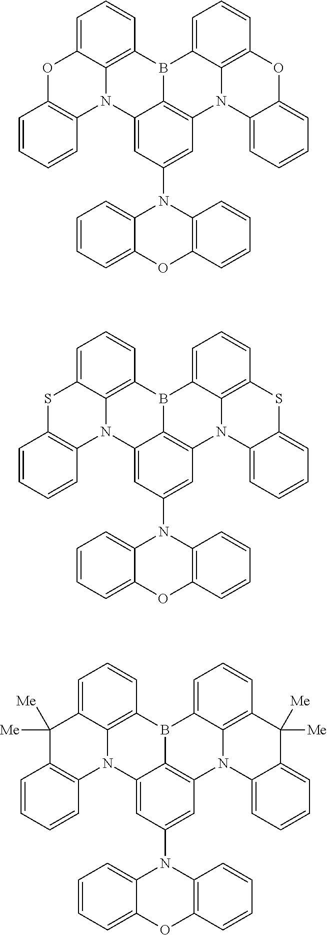 [SMILES: c1ccc2c(c1)Oc1ccccc1N2c1cc2c3c(c1)N1c4ccccc4Oc4cccc(c41)B3c1cccc3c1N2c1ccccc1O3 ; c1ccc2c(c1)Oc1ccccc1N2c1cc2c3c(c1)N1c4ccccc4Sc4cccc(c41)B3c1cccc3c1N2c1ccccc1S3 ; CC1(C)c2ccccc2N2c3cc(N4c5ccccc5Oc5ccccc54)cc4c3B(c3cccc1c32)c1cccc2c1N4c1ccccc1C2(C)C]

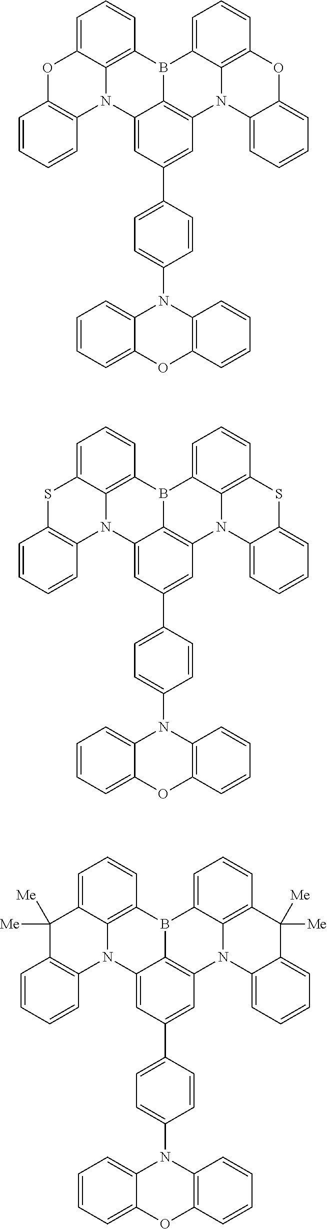 [SMILES: c1ccc2c(c1)Oc1ccccc1N2c1ccc(-c2cc3c4c(c2)N2c5ccccc5Oc5cccc(c52)B4c2cccc4c2N3c2ccccc2O4)cc1 ; c1ccc2c(c1)Oc1ccccc1N2c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccc(N5c6ccccc6Oc6ccccc65)cc4)cc4c3B(c3cccc1c32)c1cccc2c1N4c1ccccc1C2(C)C]

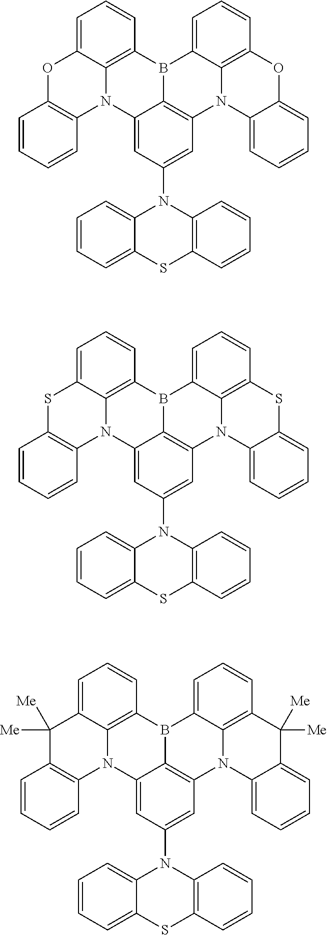 [SMILES: c1ccc2c(c1)Oc1cccc3c1N2c1cc(N2c4ccccc4Sc4ccccc42)cc2c1B3c1cccc3c1N2c1ccccc1O3 ; c1ccc2c(c1)Sc1ccccc1N2c1cc2c3c(c1)N1c4ccccc4Sc4cccc(c41)B3c1cccc3c1N2c1ccccc1S3 ; CC1(C)c2ccccc2N2c3cc(N4c5ccccc5Sc5ccccc54)cc4c3B(c3cccc1c32)c1cccc2c1N4c1ccccc1C2(C)C]

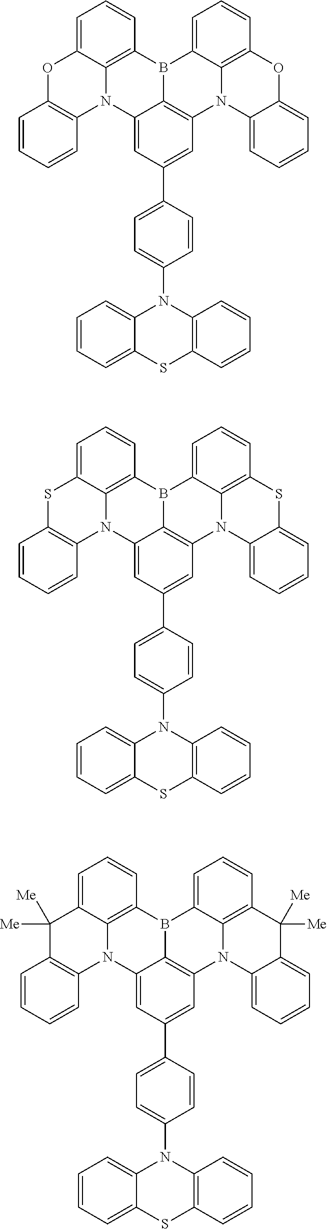 [SMILES: c1ccc2c(c1)Oc1cccc3c1N2c1cc(-c2ccc(N4c5ccccc5Sc5ccccc54)cc2)cc2c1B3c1cccc3c1N2c1ccccc1O3 ; c1ccc2c(c1)Sc1ccccc1N2c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccc(N5c6ccccc6Sc6ccccc65)cc4)cc4c3B(c3cccc1c32)c1cccc2c1N4c1ccccc1C2(C)C]

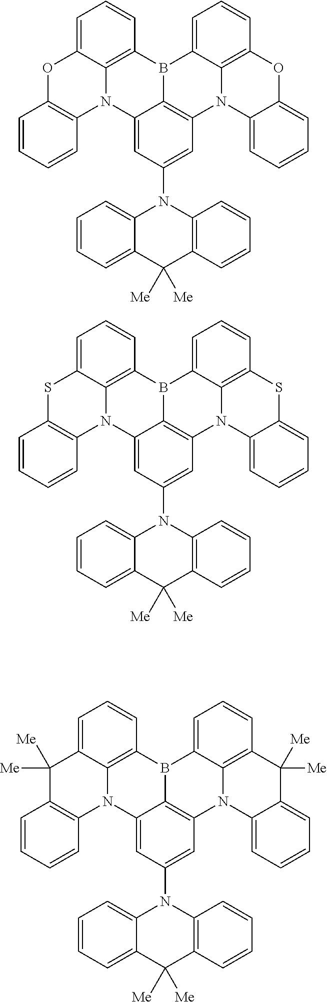

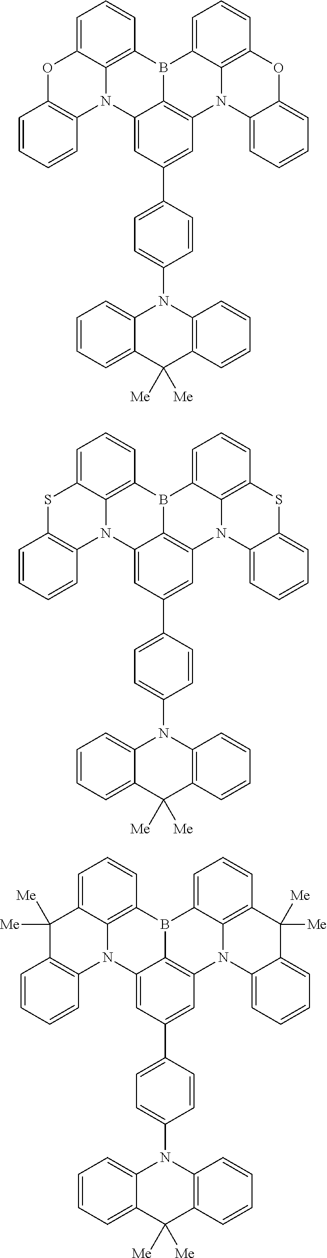 [SMILES: CC1(C)c2ccccc2N(c2ccc(-c3cc4c5c(c3)N3c6ccccc6Oc6cccc(c63)B5c3cccc5c3N4c3ccccc3O5)cc2)c2ccccc21 ; CC1(C)c2ccccc2N(c2ccc(-c3cc4c5c(c3)N3c6ccccc6Sc6cccc(c63)B5c3cccc5c3N4c3ccccc3S5)cc2)c2ccccc21 ; CC1(C)c2ccccc2N(c2ccc(-c3cc4c5c(c3)N3c6ccccc6C(C)(C)c6cccc(c63)B5c3cccc5c3N4c3ccccc3C5(C)C)cc2)c2ccccc21]

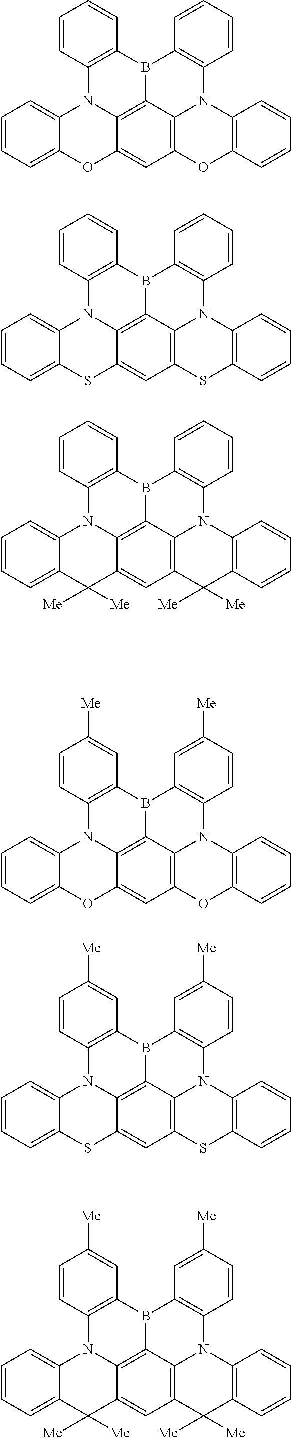

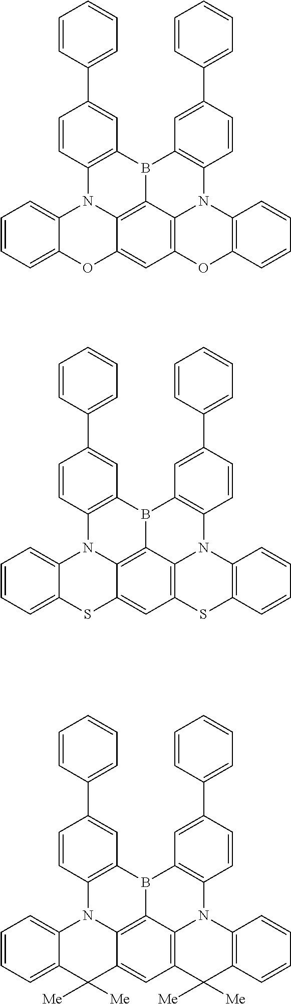 [SMILES: c1ccc(-c2ccc3c(c2)B2c4cc(-c5ccccc5)ccc4-n4c5ccccc5oc5cc6oc7ccccc7n-3c6c2c54)cc1 ; c1ccc(-c2ccc3c(c2)B2c4cc(-c5ccccc5)ccc4-n4c5ccccc5sc5cc6sc7ccccc7n-3c6c2c54)cc1 ; CC1(C)c2ccccc2N2c3ccc(-c4ccccc4)cc3B3c4cc(-c5ccccc5)ccc4N4c5ccccc5C(C)(C)c5cc1c2c3c54]

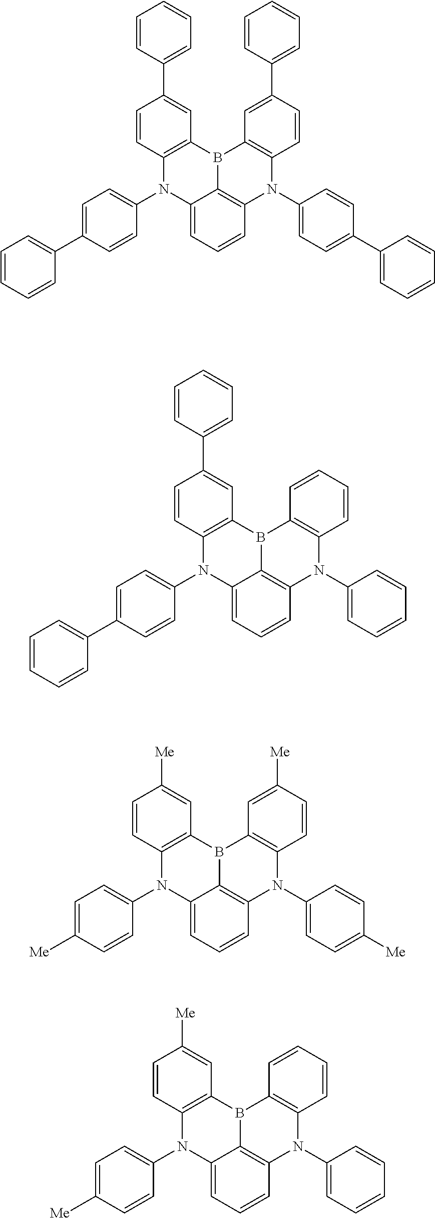 [SMILES: c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5ccccc5N(c5ccccc5)c5cccc3c54)cc2)cc1 ; Cc1ccc(N2c3ccc(C)cc3B3c4cc(C)ccc4N(c4ccc(C)cc4)c4cccc2c43)cc1 ; Cc1ccc(N2c3ccc(C)cc3B3c4ccccc4N(c4ccccc4)c4cccc2c43)cc1]

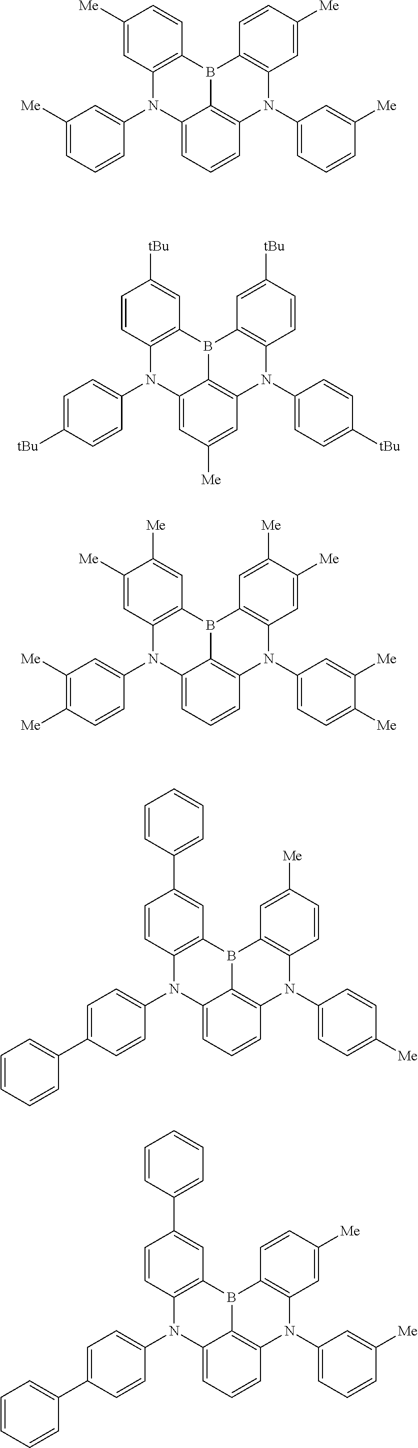 [SMILES: Cc1cccc(N2c3cc(C)ccc3B3c4ccc(C)cc4N(c4cccc(C)c4)c4cccc2c43)c1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; Cc1ccc(N2c3cc(C)c(C)cc3B3c4cc(C)c(C)cc4N(c4ccc(C)c(C)c4)c4cccc2c43)cc1C ; Cc1ccc(N2c3ccc(C)cc3B3c4cc(-c5ccccc5)ccc4N(c4ccc(-c5ccccc5)cc4)c4cccc2c43)cc1 ; Cc1cccc(N2c3cc(C)ccc3B3c4cc(-c5ccccc5)ccc4N(c4ccc(-c5ccccc5)cc4)c4cccc2c43)c1]

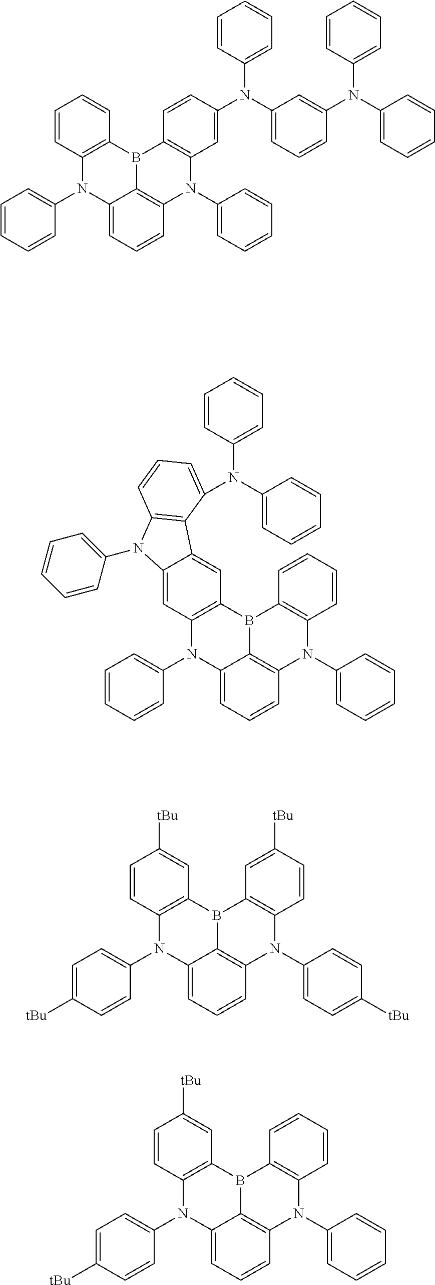 [SMILES: c1ccc(N(c2ccccc2)c2cccc(N(c3ccccc3)c3ccc4c(c3)N(c3ccccc3)c3cccc5c3B4c3ccccc3N5c3ccccc3)c2)cc1 ; c1ccc(N2c3ccccc3B3c4cc5c6c(N(c7ccccc7)c7ccccc7)cccc6n(-c6ccccc6)c5cc4N(c4ccccc4)c4cccc2c43)cc1 ; CC(C)(C)c1ccc(N2c3ccc(C(C)(C)C)cc3B3c4cc(C(C)(C)C)ccc4N(c4ccc(C(C)(C)C)cc4)c4cccc2c43)cc1 ; CC(C)(C)c1ccc(N2c3ccc(C(C)(C)C)cc3B3c4ccccc4N(c4ccccc4)c4cccc2c43)cc1]

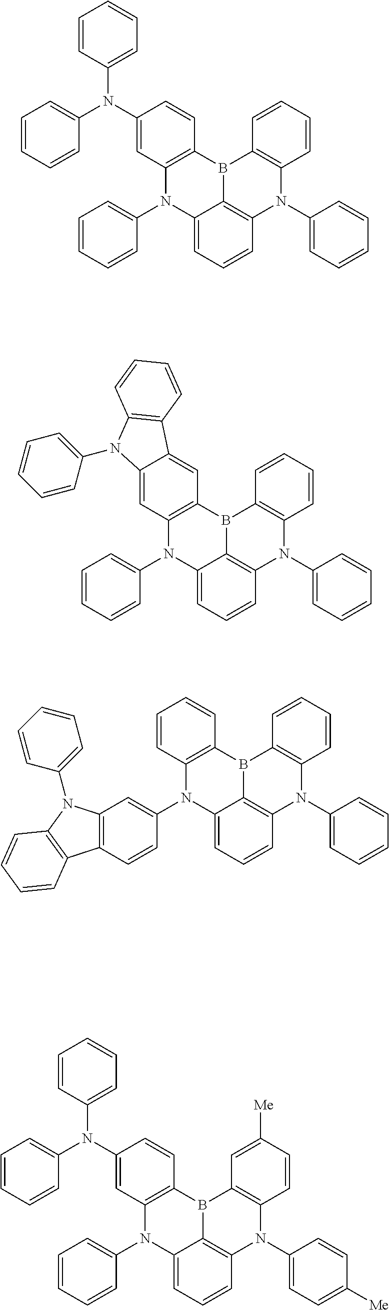 [SMILES: c1ccc(N(c2ccccc2)c2ccc3c(c2)N(c2ccccc2)c2cccc4c2B3c2ccccc2N4c2ccccc2)cc1 ; c1ccc(N2c3ccccc3B3c4cc5c6ccccc6n(-c6ccccc6)c5cc4N(c4ccccc4)c4cccc2c43)cc1 ; c1ccc(N2c3ccccc3B3c4ccccc4N(c4ccc5c6ccccc6n(-c6ccccc6)c5c4)c4cccc2c43)cc1 ; Cc1ccc(N2c3ccc(C)cc3B3c4ccc(N(c5ccccc5)c5ccccc5)cc4N(c4ccccc4)c4cccc2c43)cc1]

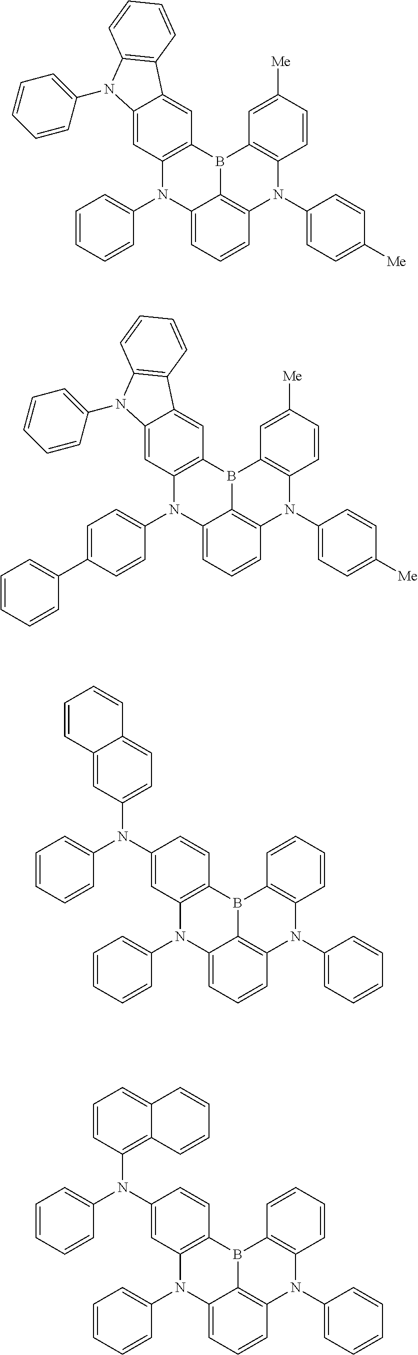 [SMILES: Cc1ccc(N2c3ccc(C)cc3B3c4cc5c6ccccc6n(-c6ccccc6)c5cc4N(c4ccccc4)c4cccc2c43)cc1 ; Cc1ccc(N2c3ccc(C)cc3B3c4cc5c6ccccc6n(-c6ccccc6)c5cc4N(c4ccc(-c5ccccc5)cc4)c4cccc2c43)cc1 ; c1ccc(N(c2ccc3c(c2)N(c2ccccc2)c2cccc4c2B3c2ccccc2N4c2ccccc2)c2ccc3ccccc3c2)cc1 ; c1ccc(N2c3ccccc3B3c4ccc(N(c5ccccc5)c5cccc6ccccc56)cc4N(c4ccccc4)c4cccc2c43)cc1]

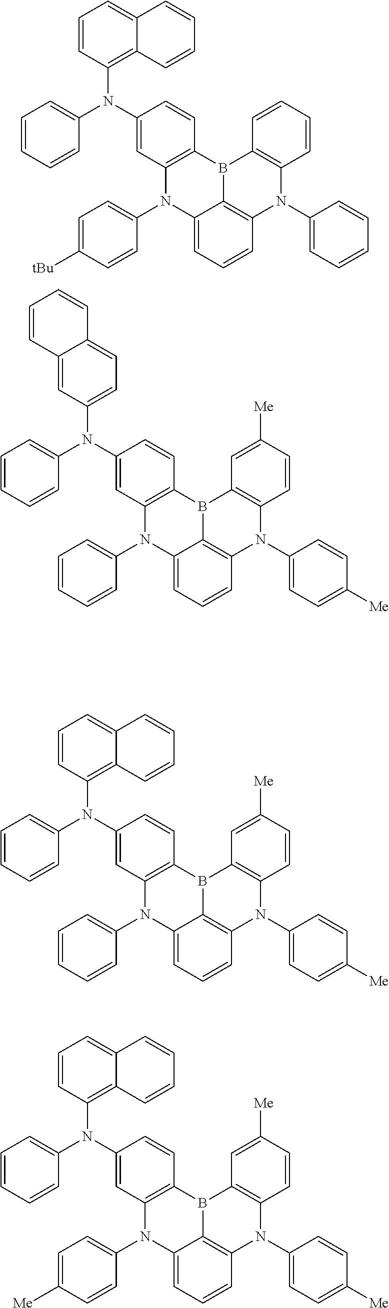

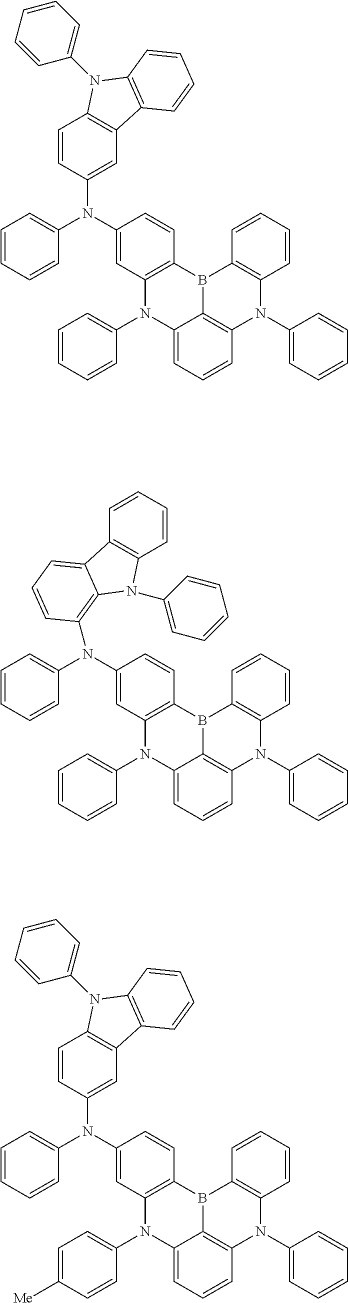 [SMILES: c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1 ; Cc1ccc(N2c3cc(N(c4ccccc4)c4ccc5c(c4)c4ccccc4n5-c4ccccc4)ccc3B3c4ccccc4N(c4ccccc4)c4cccc2c43)cc1]

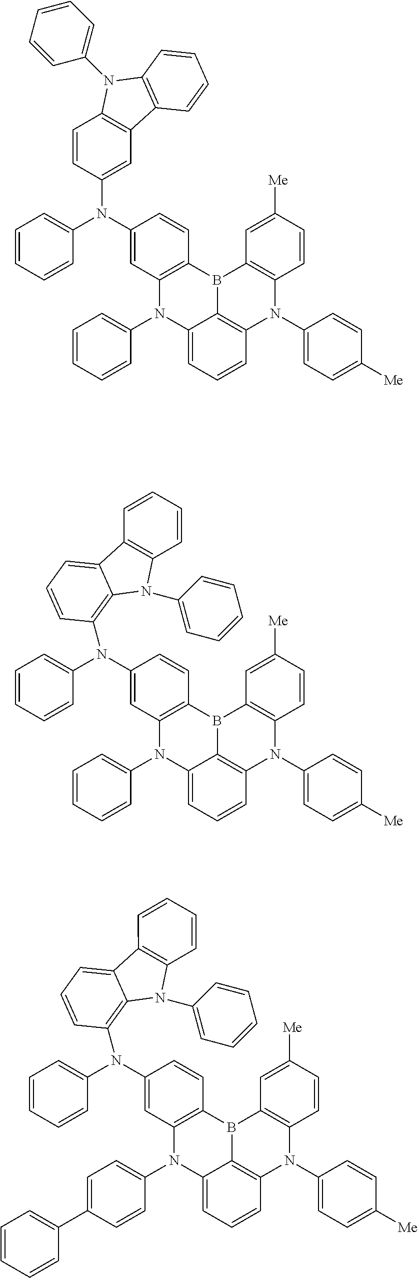 [SMILES: Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1]

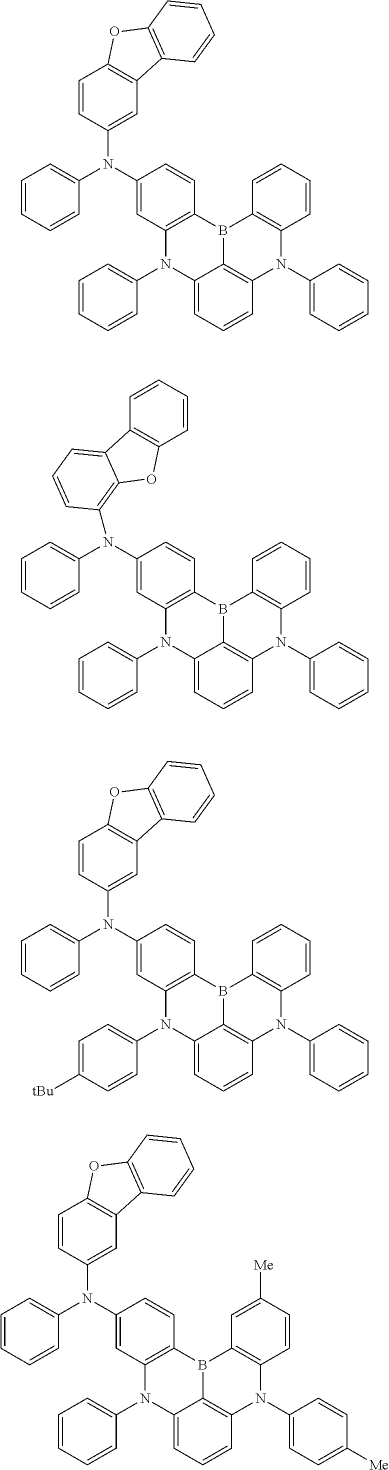 [SMILES: c1ccc(N(c2ccc3c(c2)N(c2ccccc2)c2cccc4c2B3c2ccccc2N4c2ccccc2)c2ccc3oc4ccccc4c3c2)cc1 ; c1ccc(N2c3ccccc3B3c4ccc(N(c5ccccc5)c5cccc6c5oc5ccccc56)cc4N(c4ccccc4)c4cccc2c43)cc1 ; CC(C)(C)c1ccc(N2c3cc(N(c4ccccc4)c4ccc5oc6ccccc6c5c4)ccc3B3c4ccccc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccccc4)c4cccc2c43)cc1]

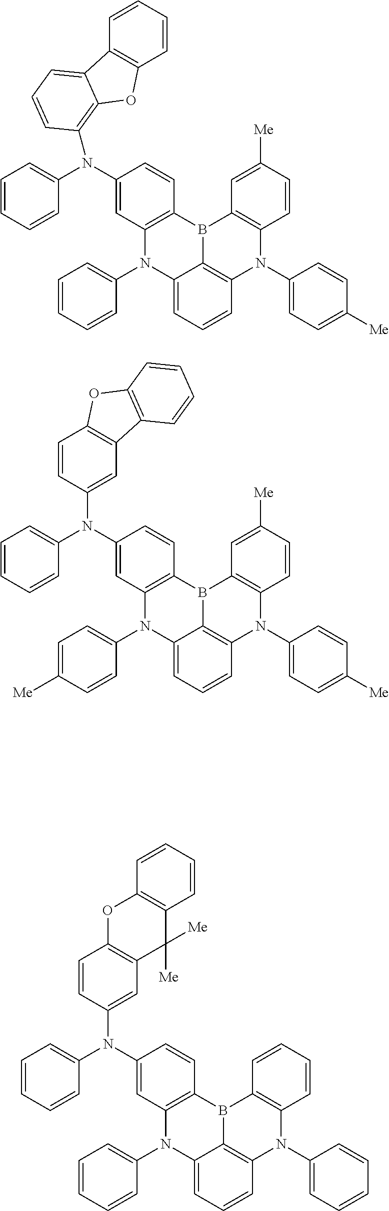

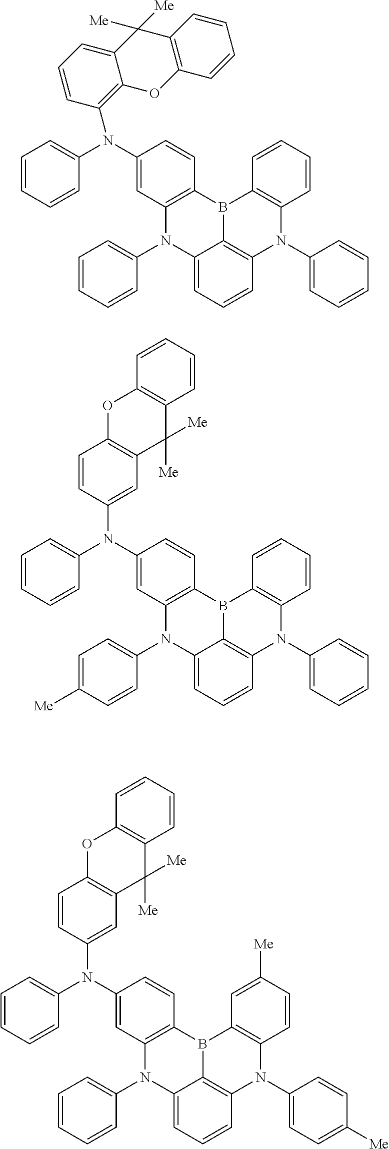 [SMILES: CC1(C)c2ccccc2Oc2c(N(c3ccccc3)c3ccc4c(c3)N(c3ccccc3)c3cccc5c3B4c3ccccc3N5c3ccccc3)cccc21 ; Cc1ccc(N2c3cc(N(c4ccccc4)c4ccc5c(c4)C(C)(C)c4ccccc4O5)ccc3B3c4ccccc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1]

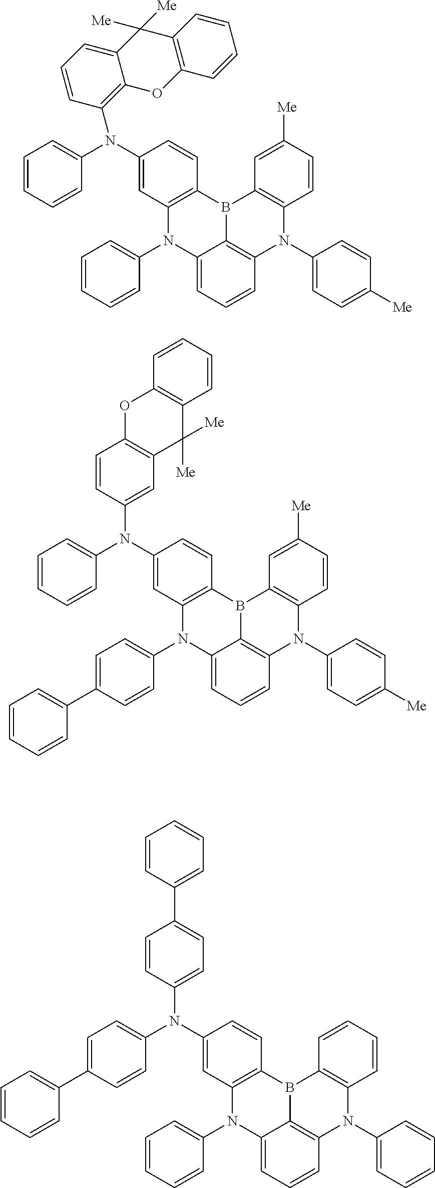 [SMILES: Cc1ccc(N2c3ccc(C)cc3B3c4ccc(N(c5ccccc5)c5cccc6c5Oc5ccccc5C6(C)C)cc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1 ; c1ccc(-c2ccc(N(c3ccc(-c4ccccc4)cc3)c3ccc4c(c3)N(c3ccccc3)c3cccc5c3B4c3ccccc3N5c3ccccc3)cc2)cc1]

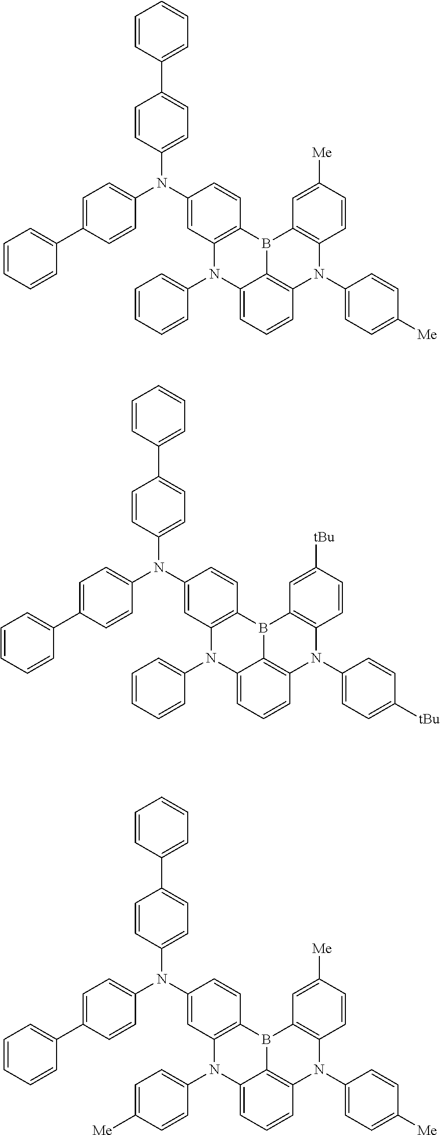 [SMILES: Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1 ; CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1 ; Cc1ccc(N2c3ccc(C)cc3B3c4ccc(N(c5ccc(-c6ccccc6)cc5)c5ccc(-c6ccccc6)cc5)cc4N(c4ccc(C)cc4)c4cccc2c43)cc1]

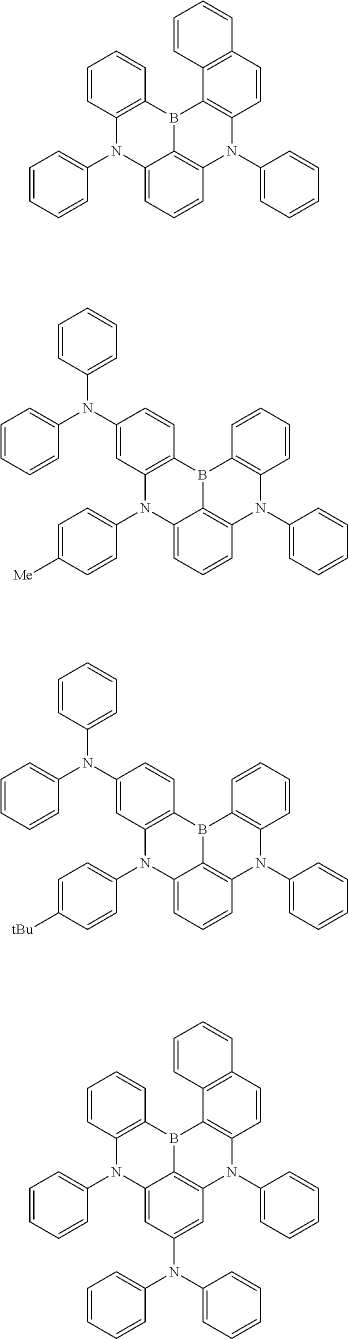 [SMILES: c1ccc(N2c3ccccc3B3c4c2cccc4N(c2ccccc2)c2ccc4ccccc4c23)cc1 ; Cc1ccc(N2c3cc(N(c4ccccc4)c4ccccc4)ccc3B3c4ccccc4N(c4ccccc4)c4cccc2c43)cc1 ; CC(C)(C)c1ccc(N2c3cc(N(c4ccccc4)c4ccccc4)ccc3B3c4ccccc4N(c4ccccc4)c4cccc2c43)cc1 ; c1ccc(N(c2ccccc2)c2cc3c4c(c2)N(c2ccccc2)c2ccc5ccccc5c2B4c2ccccc2N3c2ccccc2)cc1]

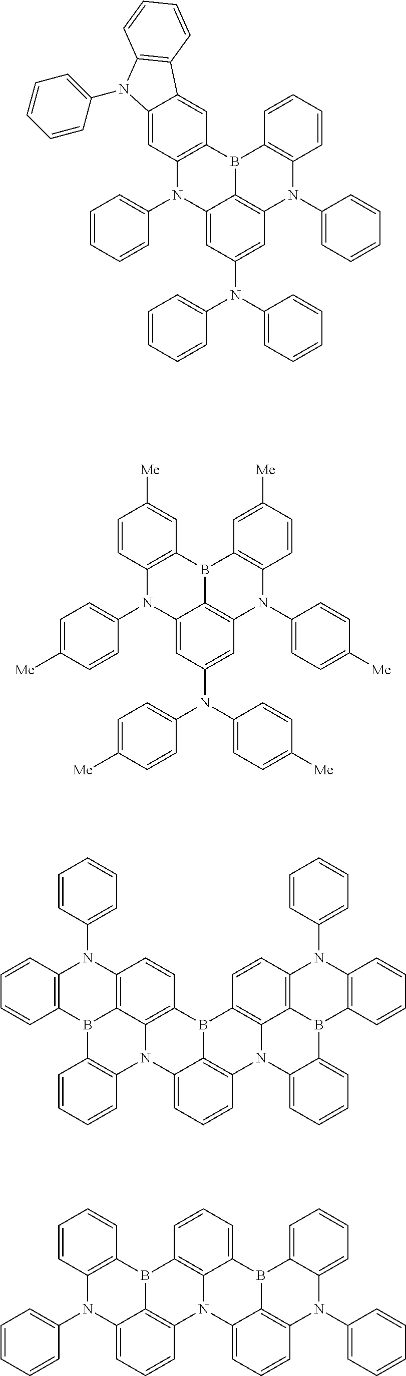 [SMILES: c1ccc(N(c2ccccc2)c2cc3c4c(c2)N(c2ccccc2)c2cc5c(cc2B4c2ccccc2N3c2ccccc2)c2ccccc2n5-c2ccccc2)cc1 ; Cc1ccc(N(c2ccc(C)cc2)c2cc3c4c(c2)N(c2ccc(C)cc2)c2ccc(C)cc2B4c2cc(C)ccc2N3c2ccc(C)cc2)cc1 ; c1ccc(N2c3ccccc3B3c4ccccc4N4c5cccc6c5B(c5ccc2c3c54)c2ccc3c4c2N6c2ccccc2B4c2ccccc2N3c2ccccc2)cc1 ; c1ccc(N2c3ccccc3B3c4cccc5c4N(c4cccc2c43)c2cccc3c2B5c2ccccc2N3c2ccccc2)cc1]

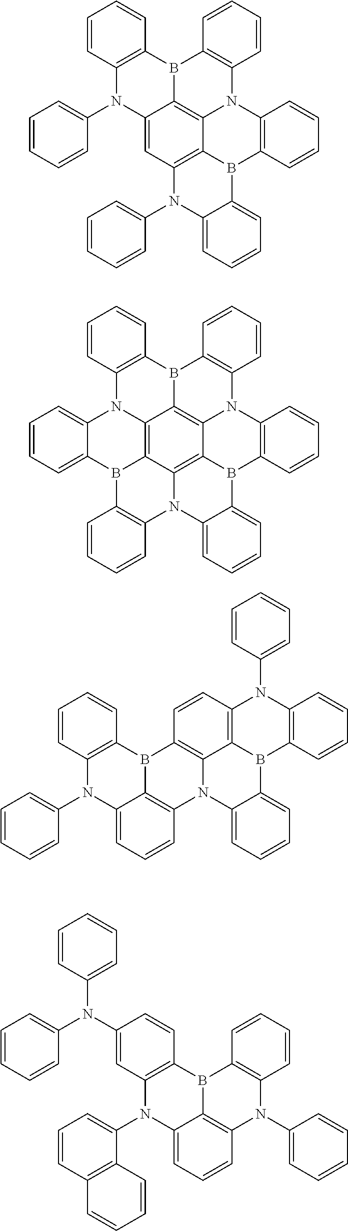

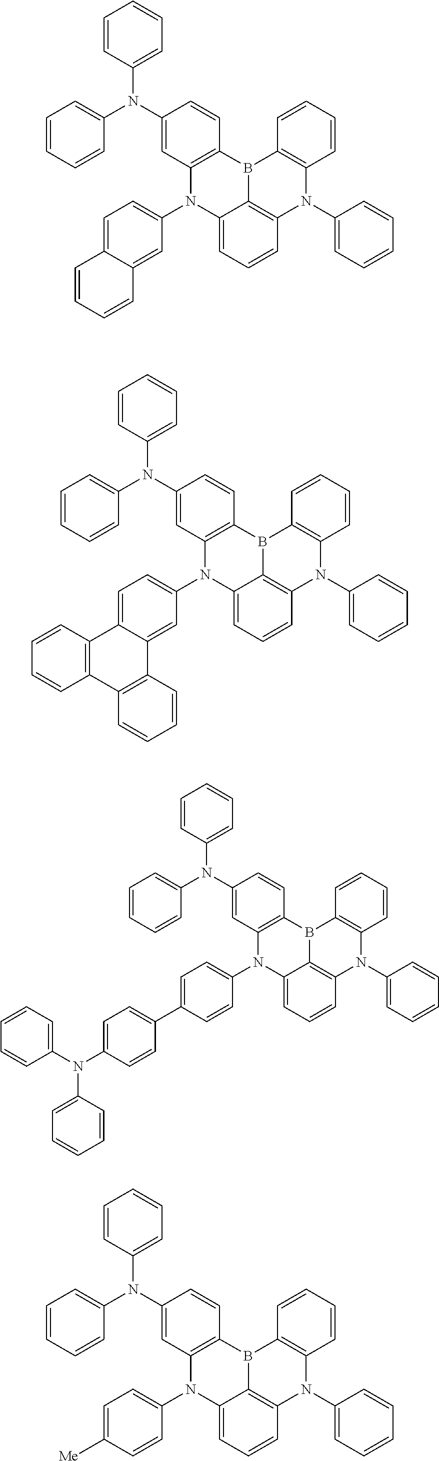 [SMILES: c1ccc(N(c2ccccc2)c2ccc3c(c2)N(c2ccc4ccccc4c2)c2cccc4c2B3c2ccccc2N4c2ccccc2)cc1 ; c1ccc(N(c2ccccc2)c2ccc3c(c2)N(c2ccc4c5ccccc5c5ccccc5c4c2)c2cccc4c2B3c2ccccc2N4c2ccccc2)cc1 ; c1ccc(N(c2ccccc2)c2ccc(-c3ccc(N4c5cc(N(c6ccccc6)c6ccccc6)ccc5B5c6ccccc6N(c6ccccc6)c6cccc4c65)cc3)cc2)cc1 ; Cc1ccc(N2c3cc(N(c4ccccc4)c4ccccc4)ccc3B3c4ccccc4N(c4ccccc4)c4cccc2c43)cc1]

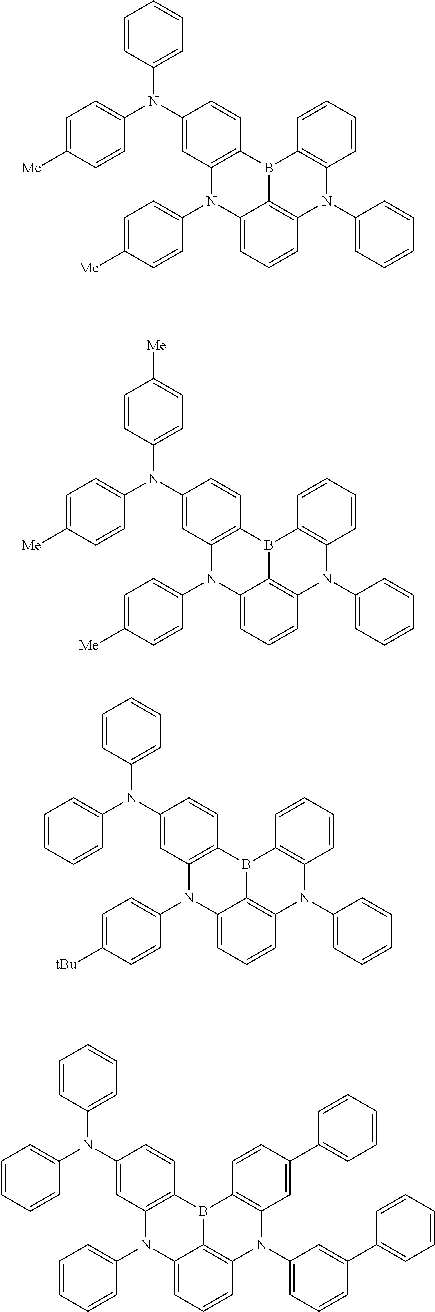 [SMILES: Cc1ccc(N(c2ccccc2)c2ccc3c(c2)N(c2ccc(C)cc2)c2cccc4c2B3c2ccccc2N4c2ccccc2)cc1 ; Cc1ccc(N(c2ccc(C)cc2)c2ccc3c(c2)N(c2ccc(C)cc2)c2cccc4c2B3c2ccccc2N4c2ccccc2)cc1 ; CC(C)(C)c1ccc(N2c3cc(N(c4ccccc4)c4ccccc4)ccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1]

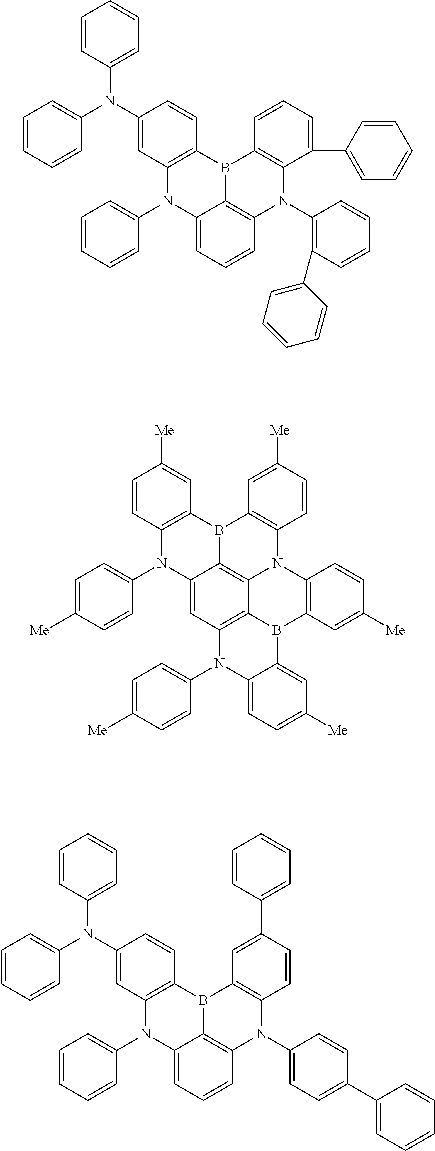 [SMILES: c1ccc(-c2ccccc2N2c3cccc4c3B(c3ccc(N(c5ccccc5)c5ccccc5)cc3N4c3ccccc3)c3cccc(-c4ccccc4)c32)cc1 ; Cc1ccc(N2c3ccc(C)cc3B3c4cc(C)ccc4N4c5ccc(C)cc5B5c6cc(C)ccc6N(c6ccc(C)cc6)c6cc2c3c4c65)cc1 ; c1ccc(-c2ccc(N3c4ccc(-c5ccccc5)cc4B4c5ccc(N(c6ccccc6)c6ccccc6)cc5N(c5ccccc5)c5cccc3c54)cc2)cc1]

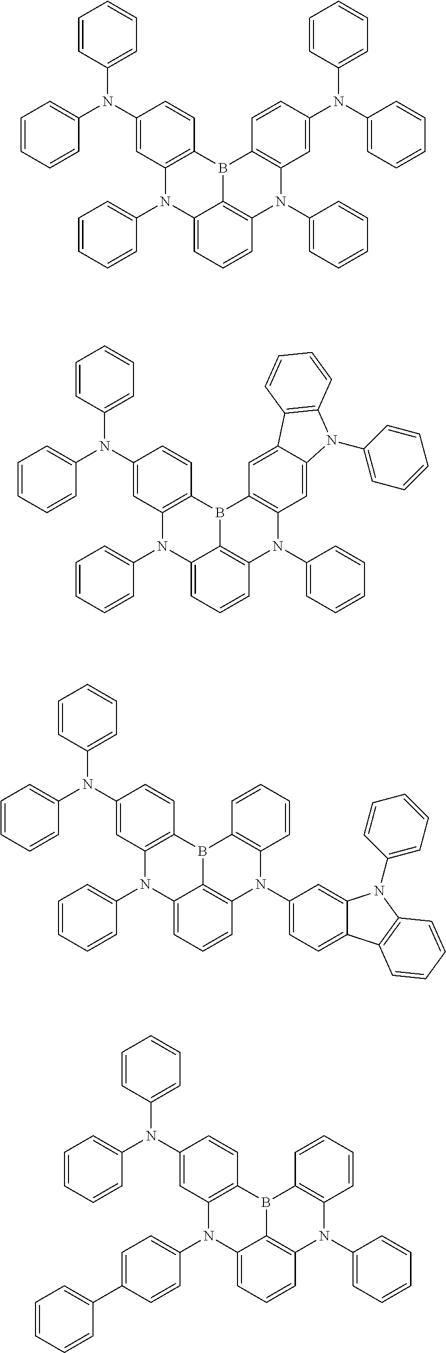 [SMILES: c1ccc(N(c2ccccc2)c2ccc3c(c2)N(c2ccccc2)c2cccc4c2B3c2ccc(N(c3ccccc3)c3ccccc3)cc2N4c2ccccc2)cc1 ; c1ccc(N(c2ccccc2)c2ccc3c(c2)N(c2ccccc2)c2cccc4c2B3c2cc3c5ccccc5n(-c5ccccc5)c3cc2N4c2ccccc2)cc1 ; c1ccc(N(c2ccccc2)c2ccc3c(c2)N(c2ccccc2)c2cccc4c2B3c2ccccc2N4c2ccc3c4ccccc4n(-c4ccccc4)c3c2)cc1 ; c1ccc(-c2ccc(N3c4cc(N(c5ccccc5)c5ccccc5)ccc4B4c5ccccc5N(c5ccccc5)c5cccc3c54)cc2)cc1]

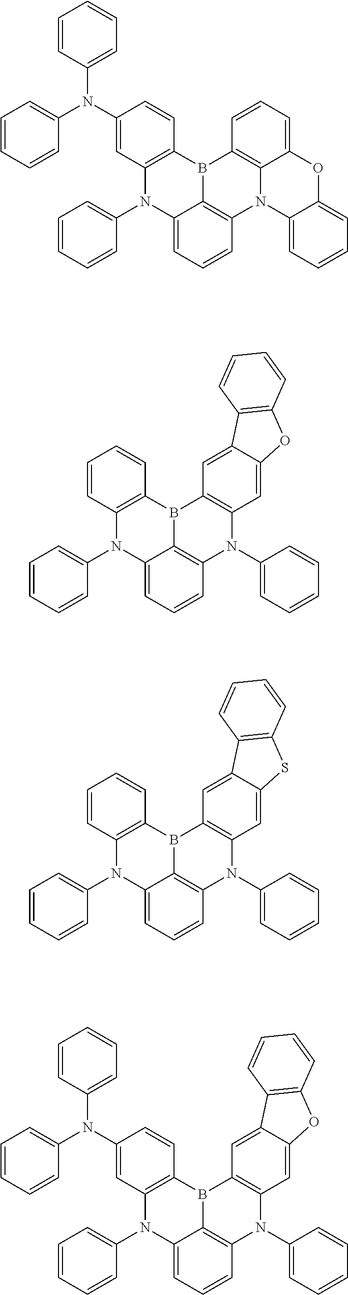 [SMILES: c1ccc(N(c2ccccc2)c2ccc3c(c2)N(c2ccccc2)c2cccc4c2B3c2cccc3c2N4c2ccccc2O3)cc1 ; c1ccc(N2c3ccccc3B3c4cc5c(cc4N(c4ccccc4)c4cccc2c43)oc2ccccc25)cc1 ; c1ccc(N2c3ccccc3B3c4cc5c(cc4N(c4ccccc4)c4cccc2c43)sc2ccccc25)cc1 ; c1ccc(N(c2ccccc2)c2ccc3c(c2)N(c2ccccc2)c2cccc4c2B3c2cc3c(cc2N4c2ccccc2)oc2ccccc23)cc1]

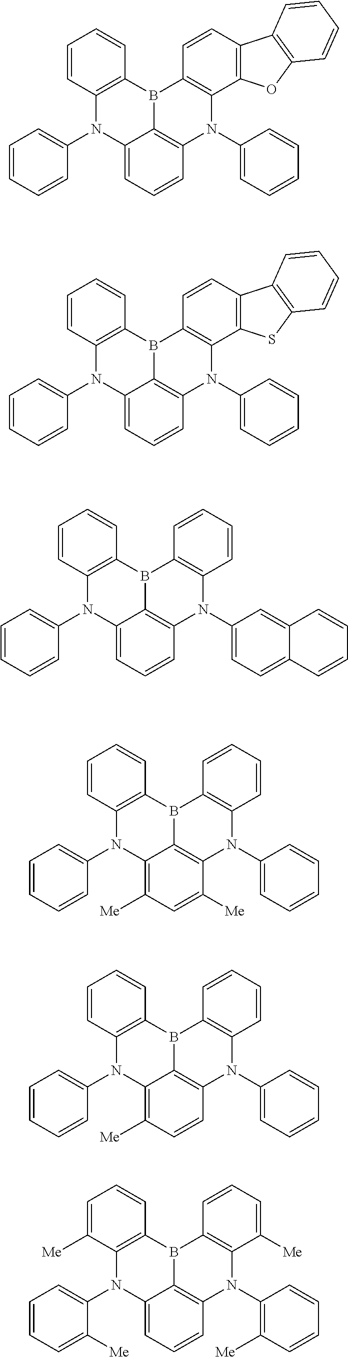 [SMILES: c1ccc(N2c3ccccc3B3c4ccc5c(oc6ccccc65)c4N(c4ccccc4)c4cccc2c43)cc1 ; c1ccc(N2c3ccccc3B3c4ccc5c(sc6ccccc65)c4N(c4ccccc4)c4cccc2c43)cc1 ; c1ccc(N2c3ccccc3B3c4ccccc4N(c4ccc5ccccc5c4)c4cccc2c43)cc1 ; Cc1cc(C)c2c3c1N(c1ccccc1)c1ccccc1B3c1ccccc1N2c1ccccc1 ; Cc1ccc2c3c1N(c1ccccc1)c1ccccc1B3c1ccccc1N2c1ccccc1 ; Cc1ccccc1N1c2cccc3c2B(c2cccc(C)c21)c1cccc(C)c1N3c1ccccc1C]

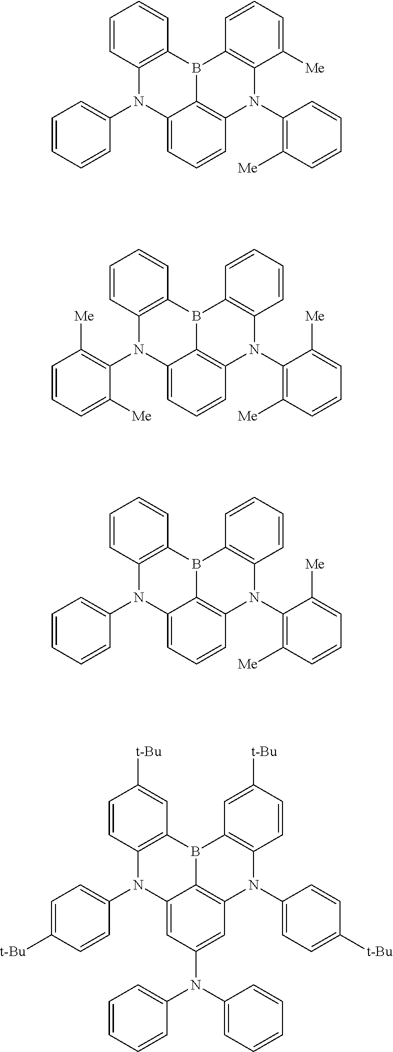 [SMILES: Cc1ccccc1N1c2cccc3c2B(c2ccccc2N3c2ccccc2)c2cccc(C)c21 ; Cc1cccc(C)c1N1c2ccccc2B2c3ccccc3N(c3c(C)cccc3C)c3cccc1c32 ; Cc1cccc(C)c1N1c2ccccc2B2c3ccccc3N(c3ccccc3)c3cccc1c32 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1]

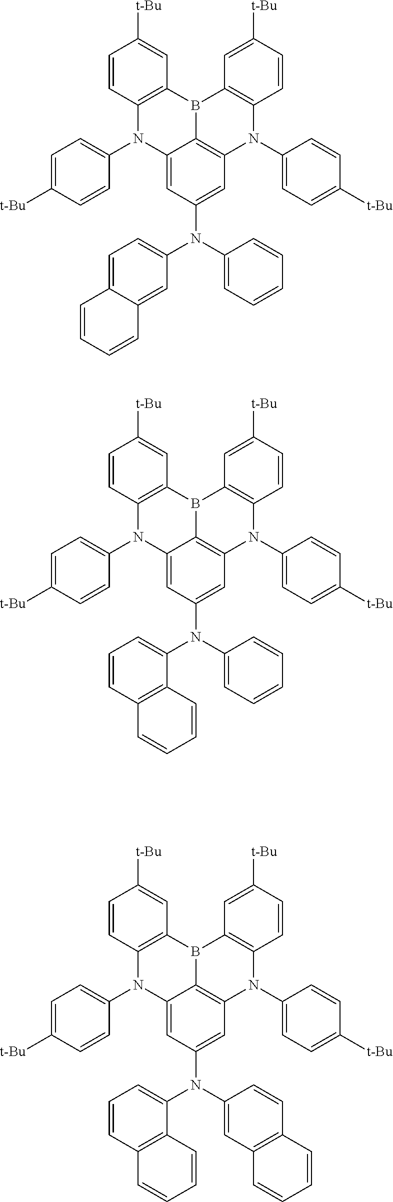 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccc6ccccc6c5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5cccc6ccccc56)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5cccc6ccccc56)cc2c43)cc1]

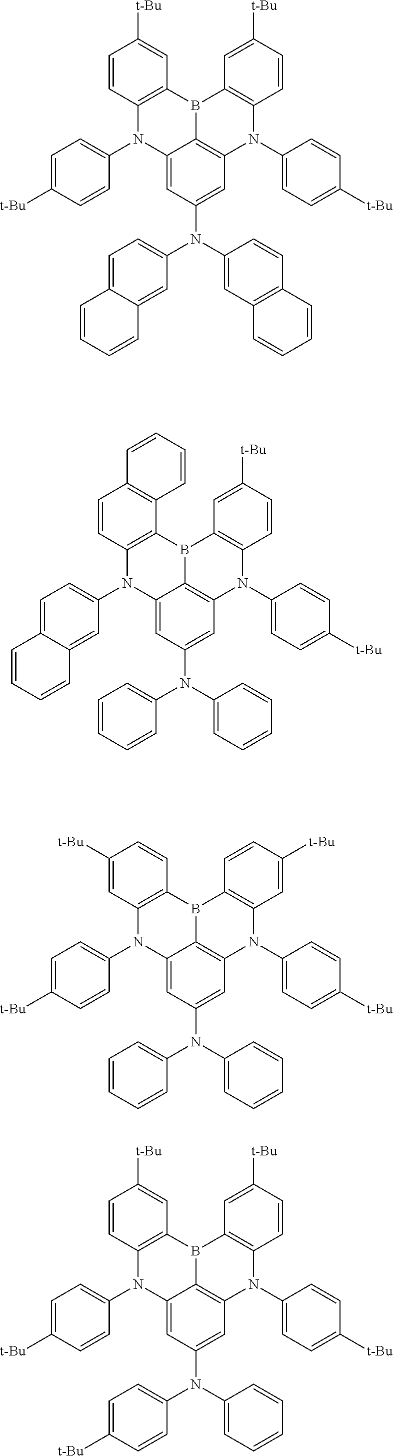

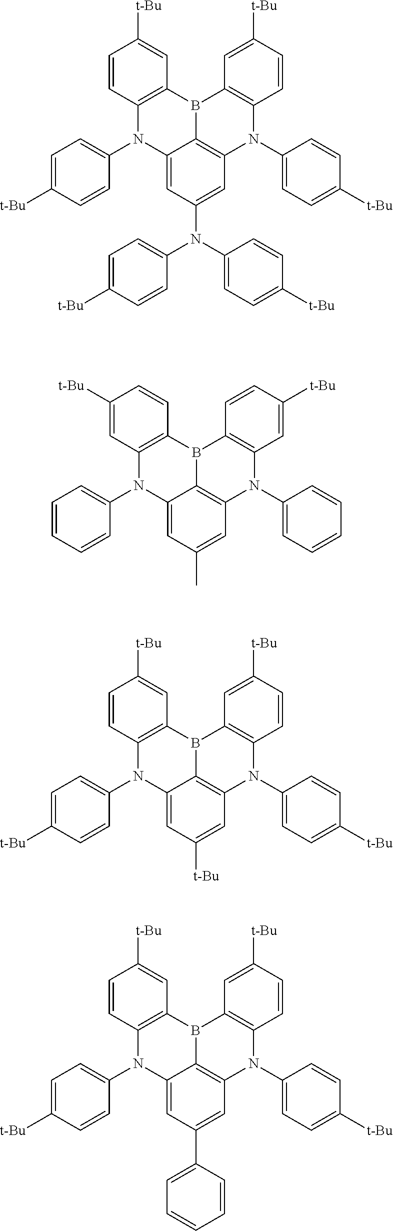 [SMILES: CC(C)(C)c1ccc(N(c2ccc(C(C)(C)C)cc2)c2cc3c4c(c2)N(c2ccc(C(C)(C)C)cc2)c2ccc(C(C)(C)C)cc2B4c2cc(C(C)(C)C)ccc2N3c2ccc(C(C)(C)C)cc2)cc1 ; Cc1cc2c3c(c1)N(c1ccccc1)c1cc(C(C)(C)C)ccc1B3c1ccc(C(C)(C)C)cc1N2c1ccccc1 ; CC(C)(C)c1ccc(N2c3ccc(C(C)(C)C)cc3B3c4cc(C(C)(C)C)ccc4N(c4ccc(C(C)(C)C)cc4)c4cc(C(C)(C)C)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccccc5)cc2c43)cc1]

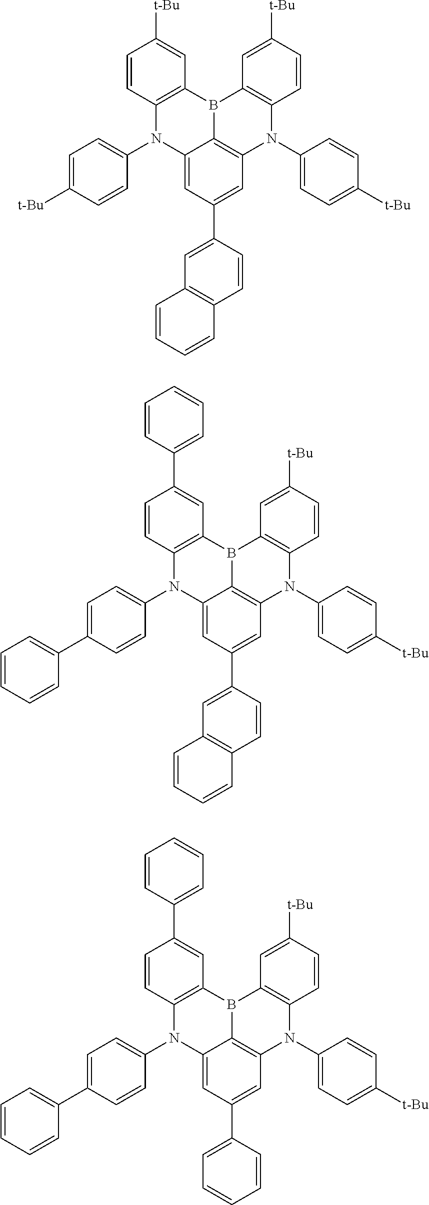

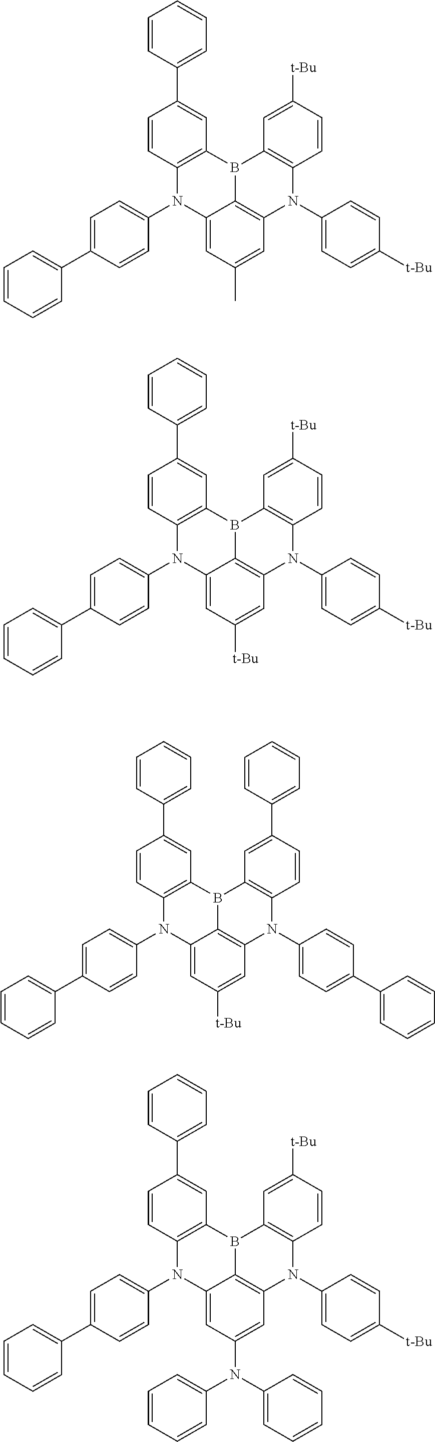 [SMILES: Cc1cc2c3c(c1)N(c1ccc(C(C)(C)C)cc1)c1ccc(C(C)(C)C)cc1B3c1cc(-c3ccccc3)ccc1N2c1ccc(-c2ccccc2)cc1 ; CC(C)(C)c1ccc(N2c3ccc(C(C)(C)C)cc3B3c4cc(-c5ccccc5)ccc4N(c4ccc(-c5ccccc5)cc4)c4cc(C(C)(C)C)cc2c43)cc1 ; CC(C)(C)c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1 ; CC(C)(C)c1ccc(N2c3ccc(C(C)(C)C)cc3B3c4cc(-c5ccccc5)ccc4N(c4ccc(-c5ccccc5)cc4)c4cc(N(c5ccccc5)c5ccccc5)cc2c43)cc1]

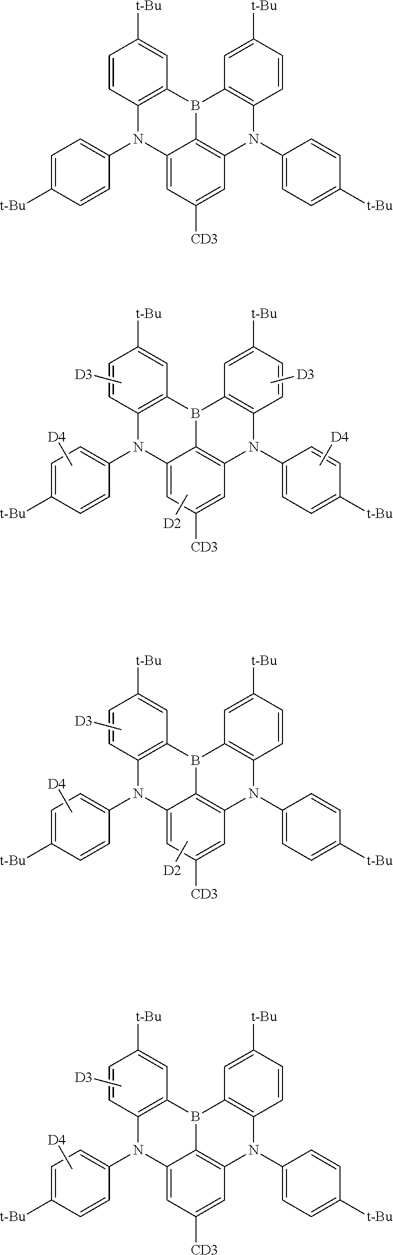

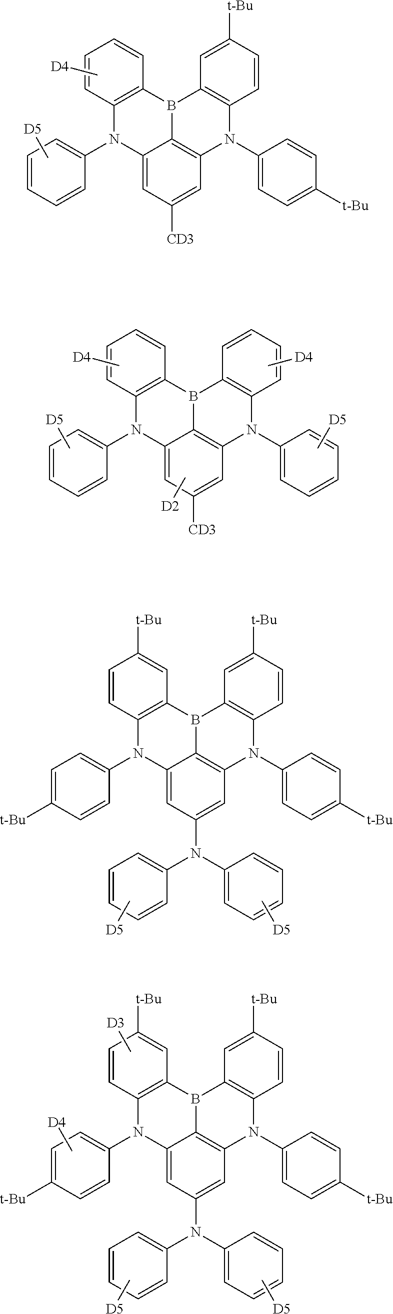

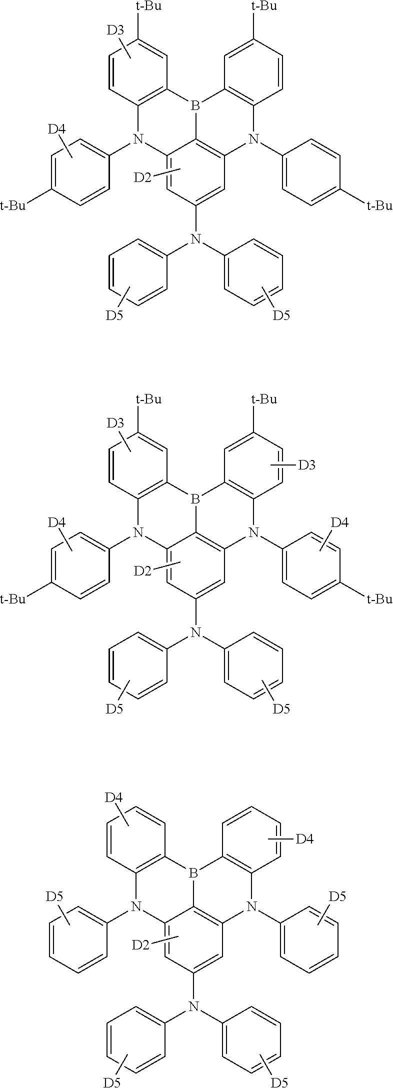

In the compounds above, D2 to D5 mean that two (2) to five (5) hydrogens each have been replaced with deuterium.

According to another embodiment, the light-emitting layer may further comprise a compound represented by the following Formula 12.

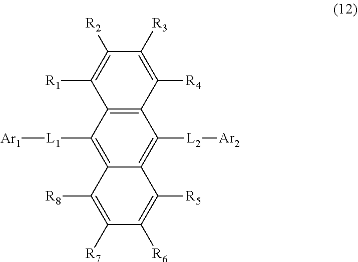

in Formula 12,
L₁ and L₂ each independently represent, a single bond, a substituted or unsubstituted (C6-C30)arylene, or a substituted or unsubstituted (5- to 30-membered)heteroarylene;
Ar₁ and Ar₂ each independently represent, a substituted or unsubstituted (C6-C30)aryl or a substituted or unsubstituted (5- to 30-membered)heteroaryl;
R₁ to R₈ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N—(Ar₄)(Ar₅);
L₄ represents a single bond, a substituted or unsubstituted (C6-C30)aryl, or a substituted or unsubstituted (3- to 30-membered)heteroaryl, or a substituted or unsubstituted divalent (C2-C30) aliphatic hydrocarbon group, or a substituted or unsubstituted fused ring of divalent (C3-C30) aliphatic ring and (C6-C30) aromatic ring; and
Ar₄ and Ar₅ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

According to one embodiment, the organic electroluminescent device according to one embodiment comprises an anode; a cathode; at least one light-emitting layer positioned between the anode and the cathode; and at least one organic layer positioned between the light-emitting layer and the cathode, wherein the organic layer includes at least one deuterated compound, and the light-emitting layer includes a plurality of host materials including at least one first host compound and at least one second host compound.

According to one embodiment, at least one of the first host compound and the second host compound may comprise deuterium, preferably all of the first host compound and the second host compound may be a deuterated compound.

According to one embodiment, at least one of the first host compound and the second host compound may be a compound containing an anthracene backbone, a triazine backbone, a dibenzofuran backbone, a dibenzothiophene backbone, or a carbazole backbone, and preferably both the first host compound and the second host compound may be compounds containing an anthracene backbone.

Hereinafter, an organic electroluminescent device to which an organic electroluminescent material including the deuterated compound represented by the aforementioned Formula 1 or 2 is applied will be described with reference to the drawings.

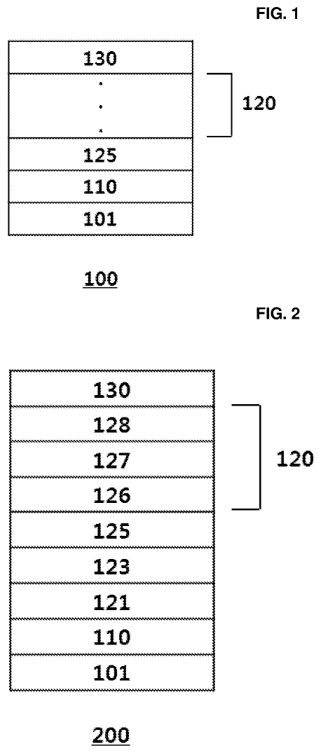

FIGS. 1 and 2 show an example of an OLED according to one embodiment, respectively.

Referring to FIG. 1, the OLED 100 according to one embodiment includes a first electrode 110 and a second electrode 130 facing each other on a substrate 101, at least one light-emitting layer 125 positioned between the first electrode 110 and the second electrode 130 and at least one organic layer 120 positioned between the light-emitting layer 125 and the second electrode 130.

According to one embodiment, the first electrode 110 may be an anode, and the second electrode 130 may be a cathode. Wherein, the first electrode 110 and the second electrode 130 may each be formed as a transmissive conductive material, a transflective conductive material, or a reflective conductive material. The organic electroluminescent device may be a top emission type, a bottom emission type, or a both-sides emission type according to the kinds of the material forming the first electrode 110 and the second electrode 130.

According to one embodiment, the organic layer 120 includes a deuterated compound according to one embodiment. The organic layer 120 may include a deuterated compound represented by Formula 1 or a deuterated compound represented by Formula 2.

According to one embodiment, the light-emitting layer 125 includes a light-emitting compound represented by Formula 11 and/or a compound represented by Formula 12. The light-emitting layer 125 is a layer in which light is emitted including a host and a dopant, and may be a single layer or a plurality of layers in which two or more layers are stacked. Wherein, the host mainly promotes recombination of electrons and holes, and has a function of confining excitons in the light-emitting layer, and the dopant has a function of efficiently emitting excitons obtained through recombination. According to one embodiment, the light-emitting layer 125 may include a compound represented by Formula 12 as a host material and a compound represented by Formula 11 as a dopant material. However, the light-emitting layer 125 does not include a delayed fluorescent compound. That is, the light-emitting compound represented by Formula 11 is not a compound that emits light in a delayed fluorescence manner. The dopant compound of the light-emitting layer 125 may be doped in an amount of less than 25% by weight, preferably, less than 17% by weight, more preferably, less than 10% by weight with respect to the total amount of host compound and the dopant compound.

Referring to FIG. 2, the organic electroluminescent device 200 according to one embodiment may include a hole transport zone between the anode and the light-emitting layer 125. The hole transport zone includes a hole injection layer 121 and a hole transport layer 123. Although not shown in the Figure, an electron blocking layer or a combination thereof may be included and used.

The hole injection layer 121 may be formed of a plurality of layers for the purpose of lowering the hole injection barrier (or hole injection voltage) from the anode to the hole transport layer 123 or the electron blocking layer, and each layer includes two compounds simultaneously. In addition, the hole injection layer 121 may be doped with p-dopant. The electron blocking layer is positioned between the hole transport layer (or hole injection layer) and the light-emitting layer 125, and blocks overflow of electrons from the light-emitting layer 125 to confine excitons in the light-emitting layer to prevent a light-emitting leakage. A plurality of layers may be used for the hole transport layer 123 or the electron blocking layer, and a plurality of compounds may be used for each layer.

The organic layer 120 including at least one deuterated compound includes a hole blocking layer 126, an electron transport layer 127, and an electron injection layer 128. The deuterated compound according to one embodiment may be included in the hole blocking layer 126 or the electron transport layer 127. A plurality of layers may also be used for the hole blocking layer 126 or the electron transport layer 127, and a plurality of compounds may be used for each layer. Also, the electron injection layer 128 may be doped with an n-dopant.

An electron buffer layer may include between the light-emitting layer 125 and the organic layer 120. In the electron buffer layer, a plurality of layers may be used for the purpose of controlling electron injection and improving interfacial characteristics between the light-emitting layer 125 and the electron injection layer 128, and each layer may contain two compounds simultaneously.

The organic electroluminescent device of the present disclosure may further include a light-emitting auxiliary layer placed between the anode and the light-emitting layer 125, or between the cathode and the light-emitting layer 125. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ (1≤X≤2), AlOₓ (1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. Also, a reductive dopant layer may be employed as a charge generating layer to prepare an organic electroluminescent device having two or more light-emitting layers and emitting white light.

The organic electroluminescent devices 100 and 200 of the present disclosure can be prepared by forming a first electrode 110 or a second electrode 130 on a substrate 101, forming an organic layer 120 using any one of dry film-forming methods such as vacuum evaporation, sputtering, plasma, ion plating methods, etc., or wet film-forming methods such as ink jet printing, nozzle printing, slot coating, spin coating, dip coating, flow coating methods, etc., and then, forming the second electrode 130 or the first electrode 110 thereon. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

In one embodiment, the present disclosure can provide display devices by using organic electroluminescent device comprising a deuterated compound represented by Formula 1 or 2 and a compound represented by Formula 2 and/or a compound represented by Formula 3. That is, it is possible to manufacture a display device or a lighting device using the organic electroluminescent device of the present disclosure. Specifically, the organic electroluminescent device of the present disclosure can be used for the manufacture of display devices such as White Organic Light Emitting Device, smartphones, tablets, notebooks, PCs, TVs, or display devices for vehicles, or lighting devices such as outdoor or indoor lighting.

Hereinafter, the preparation method of organic electroluminescent compounds according to the present disclosure will be explained with reference to the synthesis method of a representative compound or intermediate compound in order to understand the present disclosure in detail.

### [Example 1] Synthesis of Compound C-1-D18

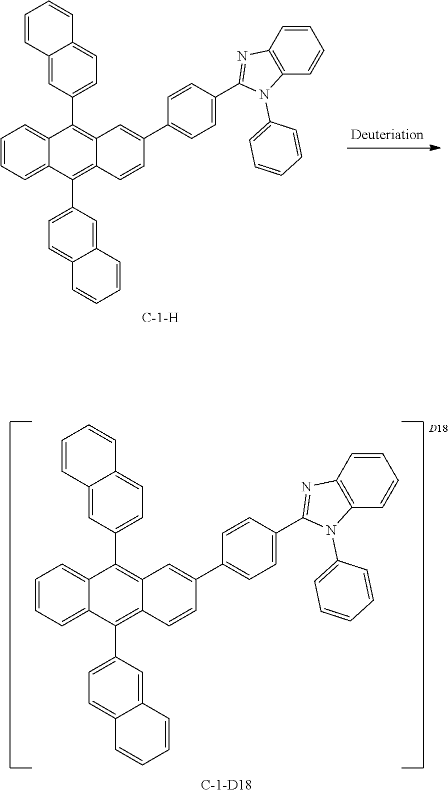

Compound C-1-H (23.0 g, 32.9 mmol), benzene-D6, and dichlorobenzene (1.8 L) were added to a flask, and then triflic acid (12 mL, 132 mmol) was added thereto at 60° C. After 3 hours, the mixture was cooled to room temperature, and 23 mL of deuterated water (D₂O) was added to the reaction mixture and stirred for 10 minutes. Next, the reaction mixture was neutralized with an aqueous solution of K₃PO₄, the organic layer was extracted with dichloromethane, the residual moisture was removed using magnesium sulfate, and the mixture was distilled under reduced pressure. Then, it was separated by column chromatography to obtain Compound C-1-D18 (wherein n is 18) (6.3 g, yield: 27%).

| Compound | MW | M.P |
|---|---|---|
| C-1-D18 | 716 | 349° C. |

Hereinafter, the preparation method of an organic electroluminescent device (OLED) comprising the aforementioned organic electroluminescent material, and the properties thereof will be explained in order to understand the present disclosure in detail.

### [Device Example 1] Producing an OLED Deposited with the Compound According to the Present Disclosure

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. The ITO substrate was mounted on a substrate holder of a vacuum vapor deposition apparatus. Then, Compound HI-1 was introduced into a cell of the vacuum vapor deposition apparatus, and Compound HT-1 was introduced into another cell of the vacuum vapor deposition apparatus. Thereafter, the two materials were evaporated at different rates and Compound HI-1 was deposited in a doping amount of 3 wt % based on the total amount of Compound HI-1 and Compound HT-1 to form a hole injection layer having a thickness of 10 nm on the ITO substrate. Next, Compound HT-1 was deposited to form a first hole transport layer having a thickness of 80 nm on the hole injection layer. Next, Compound HT-2 was introduced into another cell of the vacuum vapor deposition apparatus and was evaporated by applying an electric current to the cell, thereby depositing a second hole transport layer having a thickness of 5 nm on the first hole transport layer. After forming the hole injection layer and the hole transport layers, a light-emitting layer was formed thereon as follows: Compound H-1 was introduced into a cell of the vacuum vapor deposition apparatus as a host, and Compound D-1 was introduced into another cell as a dopant. Next, the two materials were evaporated at different rates and the dopant was deposited in a doping amount of 3 wt % based on the total amount of the host and dopant to form a light-emitting layer having a thickness of 20 nm on the second hole transport layer. Next, as an electron transport layer, Compound C-1-D18 was introduced into a cell of the vacuum vapor deposition apparatus, and Compound El-1 was introduced into another cell of the vacuum vapor deposition apparatus. The two materials were doped in a weight ratio of 50:50 to deposit an electron transport layer having a thickness of 35 nm. Next, after depositing Compound El-1 as an electron injection layer having a thickness of 2 nm on the electron transport layer, an Al cathode having a thickness of 80 nm was deposited on the electron injection layer by another vacuum vapor deposition apparatus. Thus, an OLED was produced. Each compound used for all the materials were purified by vacuum sublimation under 10⁻⁶ torr.

### [Comparative Example 1] Preparation of an OLED Comprising the Conventional Compound

An OLED was manufactured in the same manner as in Device Example 1, except that Compound C-1-H and El-1 are used as a material for electron transport layer.

The driving voltage, luminous efficiency, and the luminous color at a luminance of 1,000 nits and the time taken for luminance to decrease from 100% to 95% at a luminance of 2,020 nits (lifespan: T95) of the OLED devices of Device Example 1 and Comparative Example 1 produced as described above, are measured, and the results thereof are shown in the following Table 1.

TABLE 1

| | | | Luminous | CIE | | |
|---|---|---|---|---|---|---|
| | Electron | Driving Voltage | Efficiency | Color Coordinates | | Lifespan |
| | Transport Layer | (V) | (lm/W) | X | Y | (T95, hr) |
| Comparative | C-1-H: EI-1 | 3.7 | 5.8 | 0.130 | 0.092 | 68.0 |
| Example 1 | | | | | | |
| Device | C-1-D18: EI-1 | 3.5 | 6.3 | 0.129 | 0.094 | 68.3 |
| Example 1 | | | | | | |

### [Device Example 2] Producing OLED Comprising a Host Material According to the Present Disclosure

OLED was produced in the same manner as in Device Example 1, except that the first host compound and the second host compound listed in the following Table 2 were used at a ratio of 1:1 as a host material for the light-emitting layer.

### [Comparative Example 2] Preparation of OLED Comprising the Conventional Compound

OLED was produced in the same manner as in Comparative Example 1, except that the first host compound and the second host compound listed in the following Table 2 were used at a ratio of 1:1 as a host material for the light-emitting layer.

The driving voltage, luminous efficiency, and the luminous color at a luminance of 1,000 nits of the OLED devices of Device Example 2 and Comparative Example 2 produced as described above, are measured, and the results thereof are shown in the following Table 2, respectively.

TABLE 2

| | | | Electron | Driving | Luminous | Color | |
|---|---|---|---|---|---|---|---|
| | | | Transport | Voltage | Efficiency | Coordinates | |
| | First Host | Second Host | Layer | [V] | [lm/W] | x | y |
| Comparative | H-2-D24 | H-3-D13 | C-1-H: EI-1 | 4.0 | 5.3 | 0.129 | 0.096 |
| Example 2 | | | | | | | |
| Device | H-2-D24 | H-3-D13 | C-1-D18: EI-1 | 3.8 | 5.8 | 0.129 | 0.091 |
| Example 2 | | | | | | | |

From Tables 1 and 2 above, it can be confirmed that the organic electroluminescent device including the deuterated compound according to the present disclosure in the light-emitting layer and/or the electron transport layer exhibits low driving voltage and high luminous efficiency compared to conventional organic electroluminescent devices.

The compounds used in Device Examples and Comparative Examples are specifically shown in the following Table 3.

TABLE 3

| | |
|---|---|
| Hole Injection Layer/Hole Transport Layer | 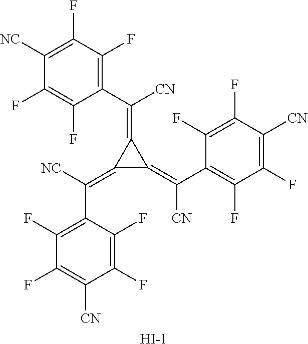 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 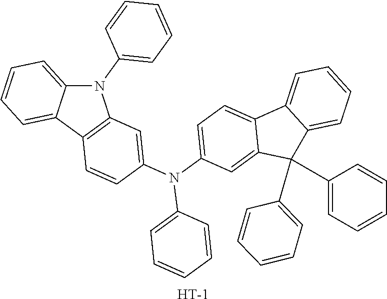 |
| | 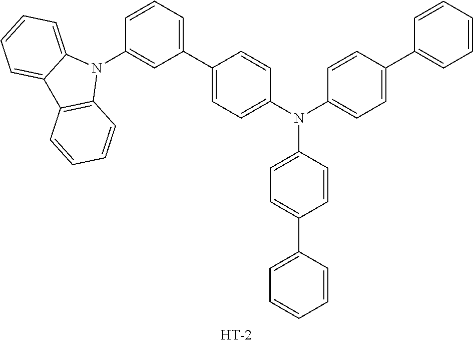 |
| Light-Emitting Layer | 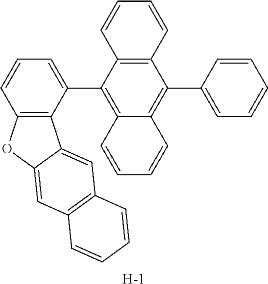 |
| | 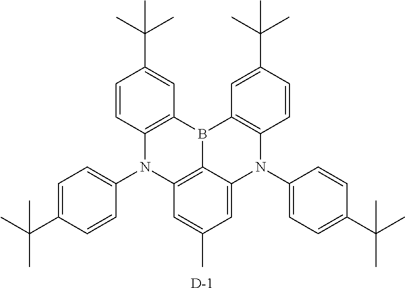 |
| | 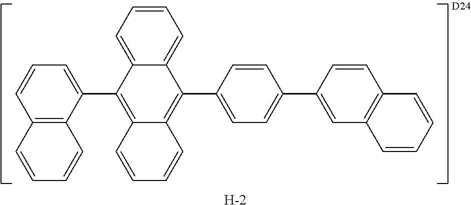 |
| | 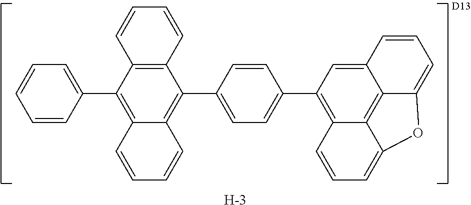 |
| | 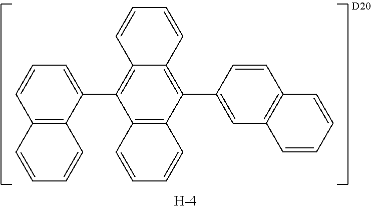 |
| | 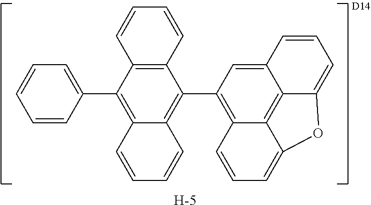 |
| Electron Transport Layer/ Electron Injection Layer | 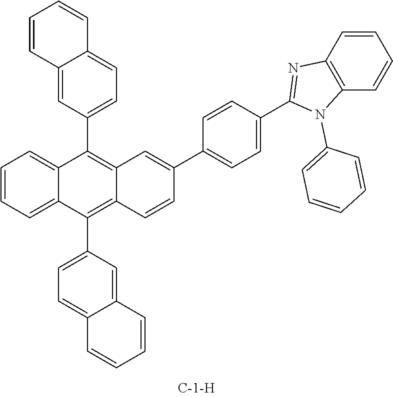 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1] |
| | 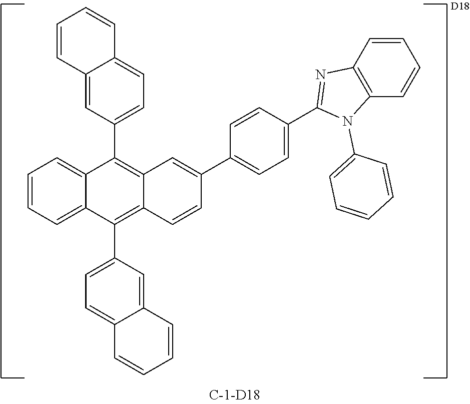 |
| | 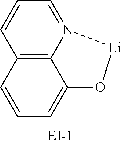 |

<Description of Symbol>

| | |
|---|---|
| 100, 200: organic electroluminescent | |
| device | |
| 101: Substrate | 110: First Electrode |
| 120: Organic Layer | 121: Hole Injection Layer |
| 123: Hole Transport Layer | 125: Light-Emitting Layer |
| 126: Hole Blocking Layer | 127: Electron Transport Layer |
| 128: Electron Injection Layer | 130: Second Electrode |

1. An organic electroluminescent device comprising
an anode;
a cathode;
at least one light-emitting layer positioned between the anode and the cathode; and
at least one organic layer positioned between the light-emitting layer and the cathode,
wherein the organic layer includes at least one deuterated compound, and
wherein the light-emitting layer includes a light-emitting compound including an iridium (Ir), platinum (Pt), or boron (B) compound, but does not include a delayed fluorescent compound.

2. The organic electroluminescent device according to claim 1, wherein the degree of deuteriumization of the deuterated compounds is 30% to 100%.

3. The organic electroluminescent device according to claim 1, wherein the degree of deuteriumization of the deuterated compounds is 55% to 100%.

4. The organic electroluminescent device according to claim 1, wherein the degree of deuteriumization of the deuterated compounds is 30% to 99%.

5. The organic electroluminescent device according to claim 1, wherein the deuterated compound is represented by the following Formula 1. 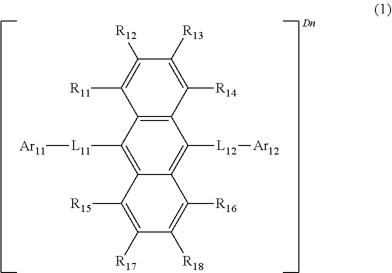
wherein,
L₁₁ and L₁₂ each independently represent, a single bond, a substituted or unsubstituted (C6-C30)arylene, or a substituted or unsubstituted (3- to 30-membered)heteroarylene;
Ar₁₁ and Ar₁₂ each independently represent, a substituted or unsubstituted (C6-C30)aryl or a substituted or unsubstituted (3- to 30-membered)heteroaryl;
R₁₁ to R₁₈ each independently represent, hydrogen, deuterium, haloen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
at least one of R₁₈ and R₁₄ to R₁₆ is deuterium; and
Dn means that n number of hydrogens is replaced with deuterium, wherein the upper limit of n is determined by the number of hydrogens that can be substituted in each compound.

6. The organic electroluminescent device according to claim 5, wherein at least one of R₁₁ to R₁₈, Ar₁₁, and Ar₁₂ is represented by the following Formula 1-1 or 1-2. 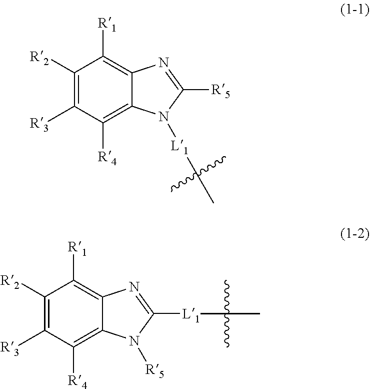
wherein
L′₁ represents a single bond, a substituted or unsubstituted (C6-C30)arylene, or a substituted or unsubstituted (3- to 30-membered)heteroarylene; and
R′₁ to R′₅ each independently represent, hydrogen, deuterium, a substituted or unsubstituted (C1-C30)alkyl, a substituted or unsubstituted (C6-C30)aryl, or a substituted or unsubstituted (3- to 30-membered)heteroaryl.

7. The organic electroluminescent device according to claim 1, wherein the deuterated compound is represented by the following Formula 2. 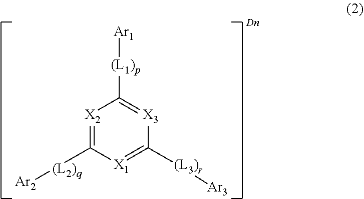
wherein
X₁ to X₃ each independently represent, CR′ or N; provided that at least two of X₁ to X₃ are N;
R′ represents hydrogen or deuterium;
L₁ to L₃ each independently represent, a single bond, a substituted or unsubstituted (C6-C30)arylene, or a substituted or unsubstituted (3- to 30-membered)heteroarylene;
Ar₁ to Ar₃ each independently represent, a substituted or unsubstituted (C6-C30)aryl, or a substituted or unsubstituted (3- to 30-membered)heteroaryl; provided that at least one of Ar₁ to Ar₃ comprises deuterium;
p, q, and r each independently represent, an integer of 1 to 3, when p, q, and r are an integer of 2 or more, each of L₁ to L₃ may be the same or different; and
Dn means that n number of hydrogens is replaced with deuterium, wherein the upper limit of n is determined by the number of hydrogens that can be substituted in each compound.

8. The organic electroluminescent device according to claim 1, wherein the light-emitting layer comprises a light-emitting compound represented by the following Formula 11. 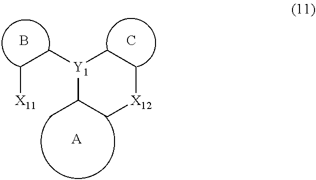
wherein
Ring A, Ring B, and Ring C each independently represent, a substituted or unsubstituted (C6-C30)aryl or a substituted or unsubstituted (3- to 50-membered)heteroaryl;
Y₁ represents B;
X₁₁ and X₁₂ each independently represent, NRₐ, O, or S;
Rₐ represents hydrogen, deuterium, halogen, cyano, a substituted or unsubstituted (C1-C30)alkyl, a substituted or unsubstituted (C6-C30)aryl, a substituted or unsubstituted (3 to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N—(Ar₄)(Ar₅); or Rₐ may be linked to at least one of Ring A, Ring B, and Ring C to form a ring(s);
L₄ represents a single bond, a substituted or unsubstituted (C6-C30)aryl, a substituted or unsubstituted (3- to 30-membered)heteroaryl, or a substituted or unsubstituted divalent (C2-C30) aliphatic hydrocarbon group, or a substituted or unsubstituted fused ring of divalent (C3-C30) aliphatic ring and (C6-C30) aromatic ring; and
Ar₄ and Ar₅ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

9. The organic electroluminescent device according to claim 1, wherein the light-emitting layer further comprises a compound represented by the following Formula 12. 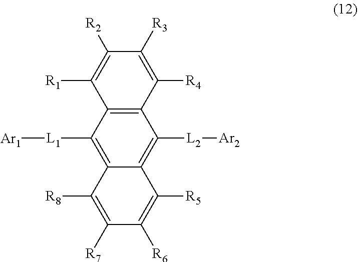
wherein
L₁ and L₂ each independently represent, a single bond, a substituted or unsubstituted (C6-C30)arylene, or a substituted or unsubstituted (5- to 30-membered)heteroarylene;
Ar₁ and Ar₂ each independently represent, a substituted or unsubstituted (C6-C30)aryl or a substituted or unsubstituted (5- to 30-membered)heteroaryl;
R₁ to R₈ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N—(Ar₄)(Ar₅);
L₄ represents a single bond, a substituted or unsubstituted (C6-C30)aryl, or a substituted or unsubstituted (3- to 30-membered)heteroaryl, or a substituted or unsubstituted divalent (C2-C30) aliphatic hydrocarbon group, or a substituted or unsubstituted fused ring of divalent (C3-C30) aliphatic ring and (C6-C30) aromatic ring; and
Ar₄ and Ar₅ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

10. The organic electroluminescent device according to claim 1, wherein the deuterated compound is selected from the following compounds: 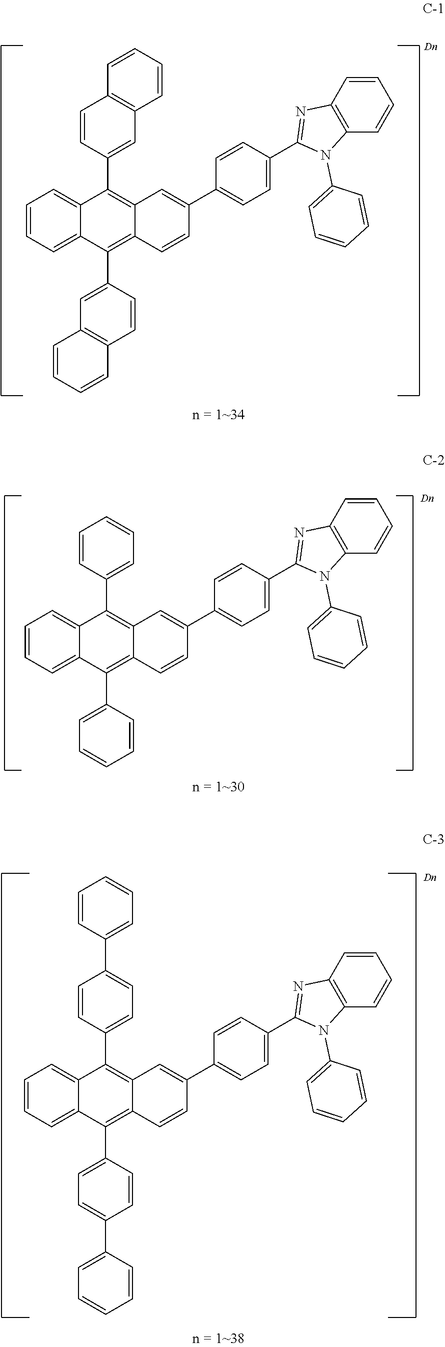 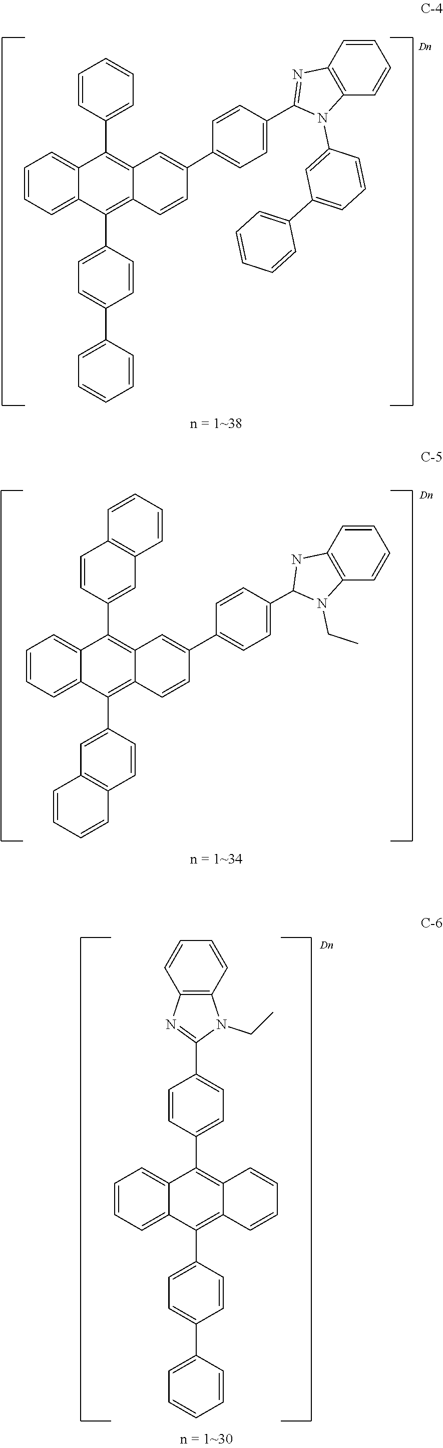 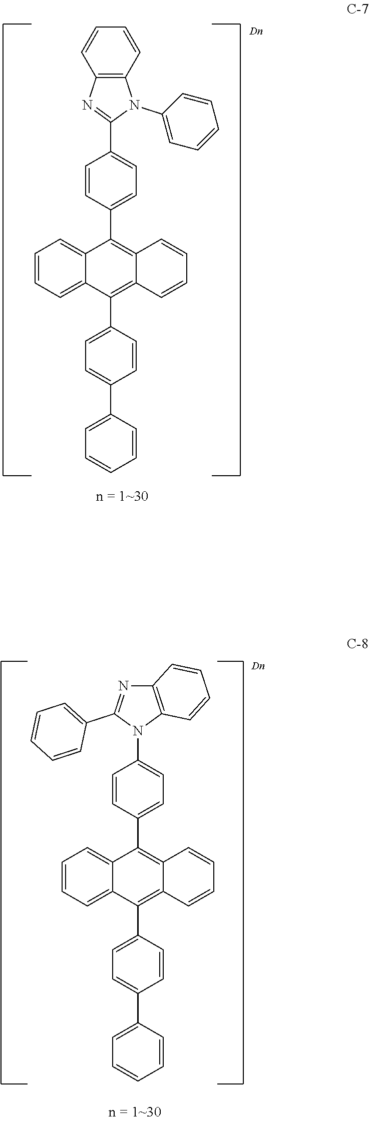 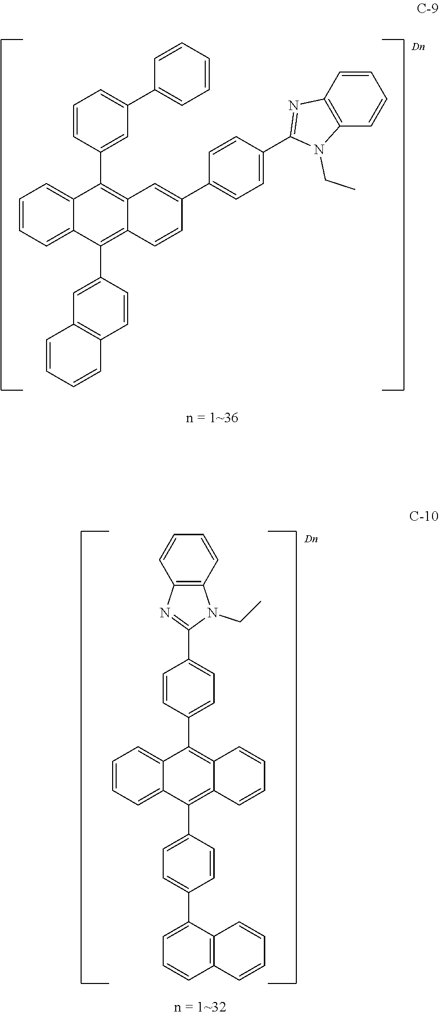 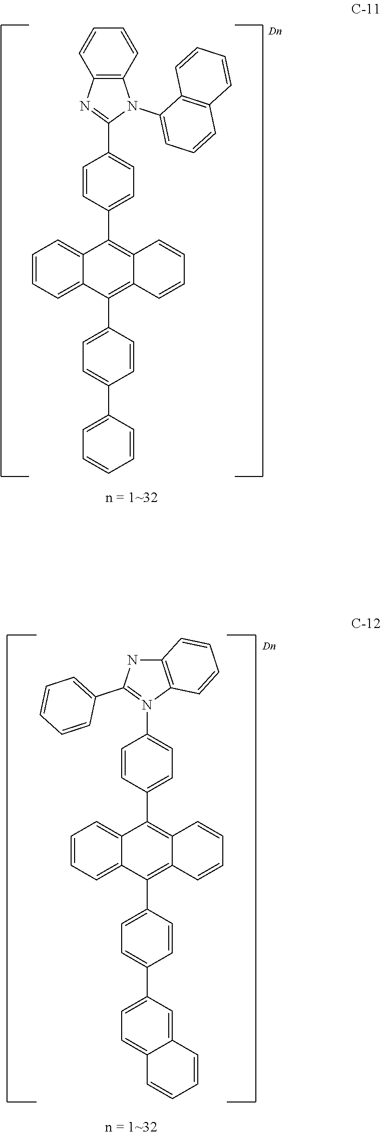 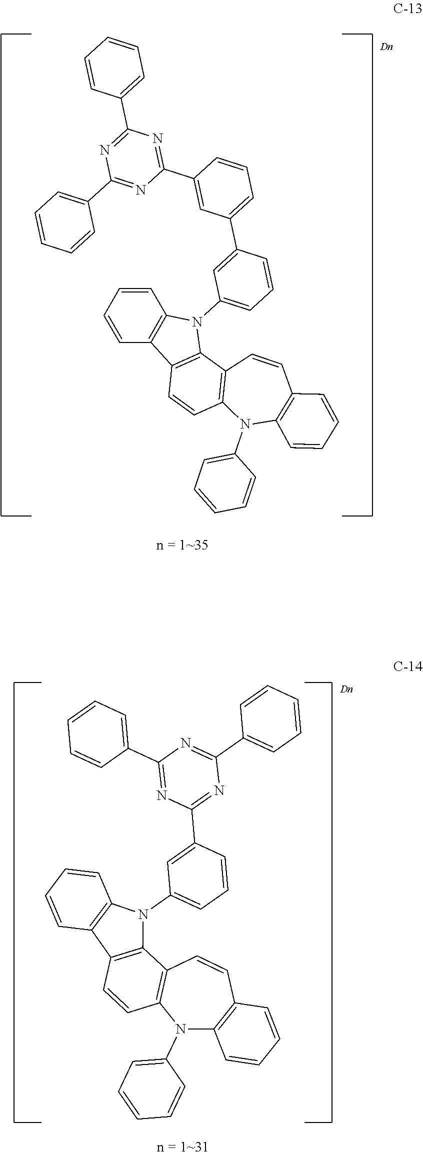 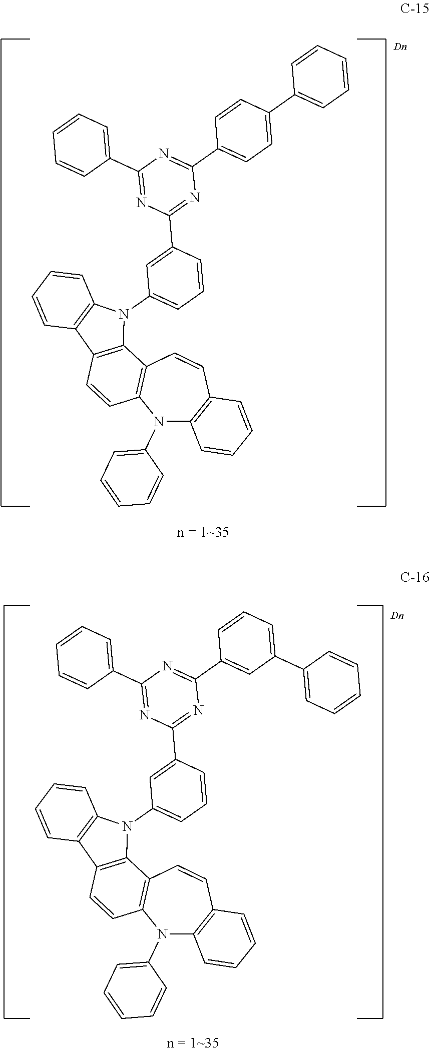 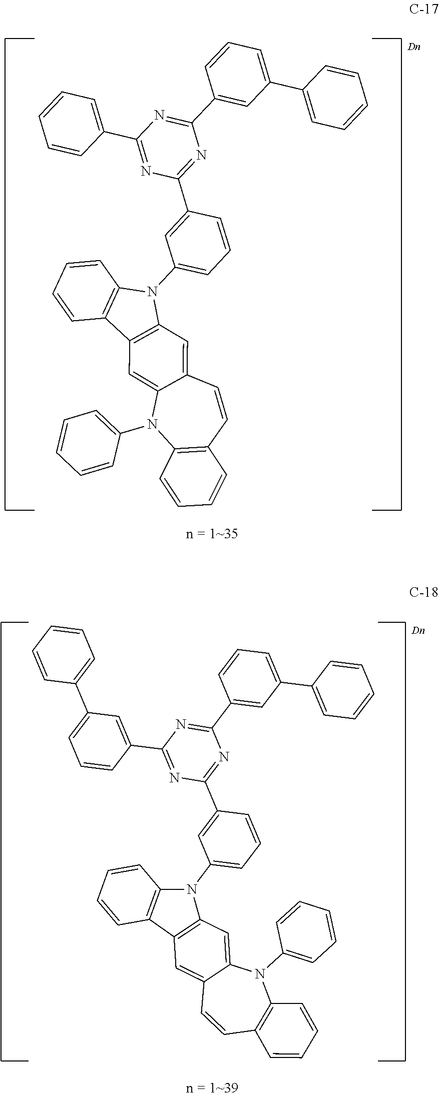 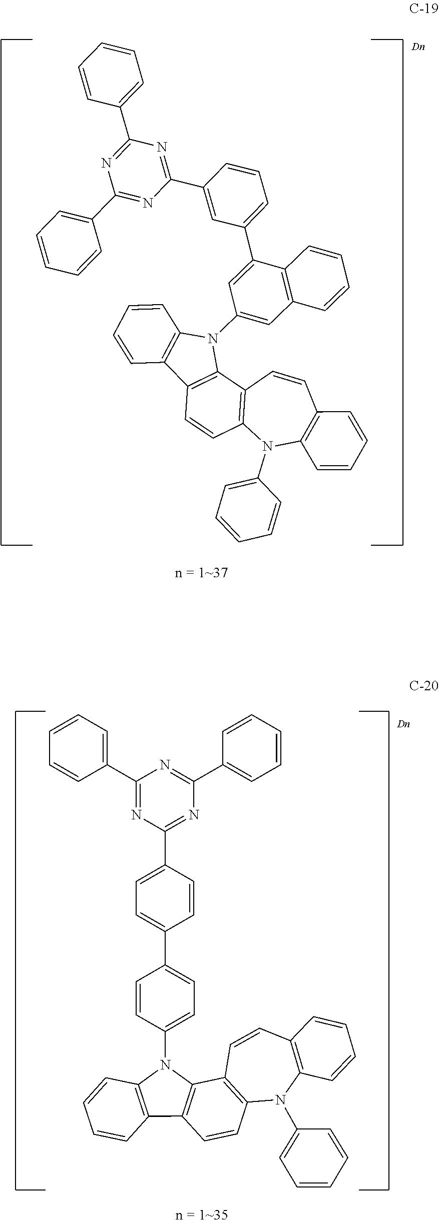 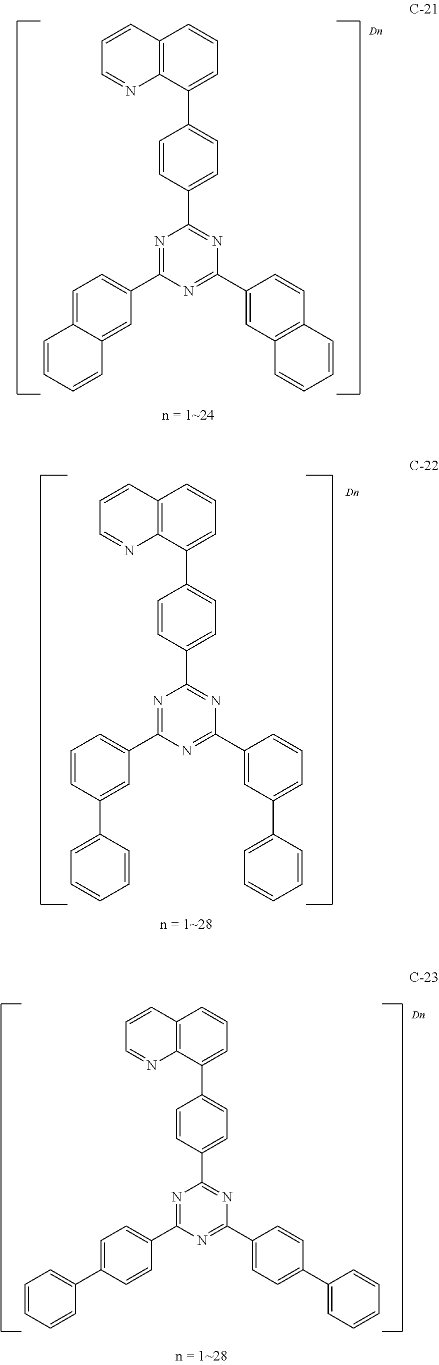 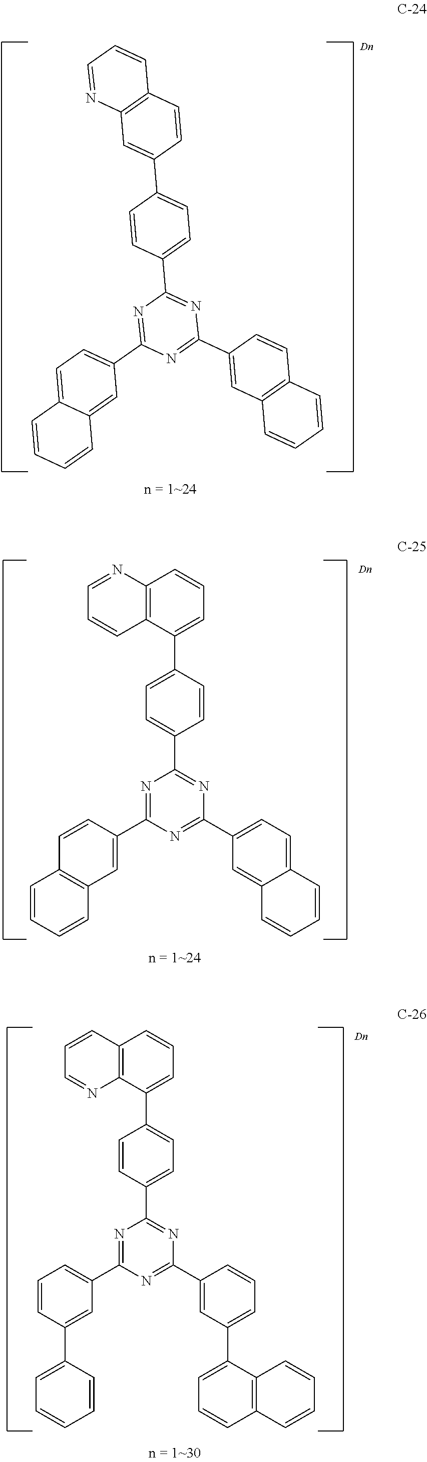 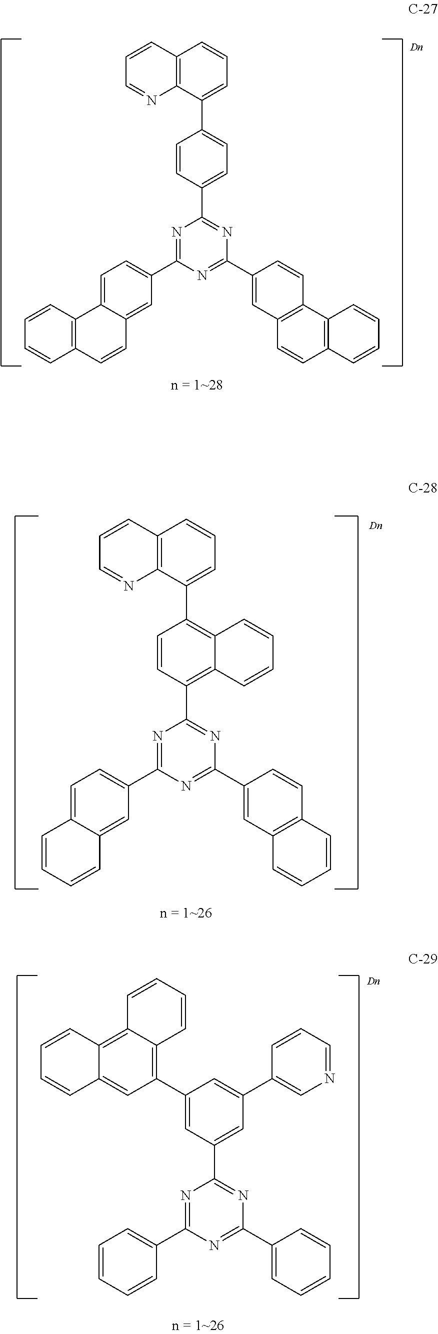 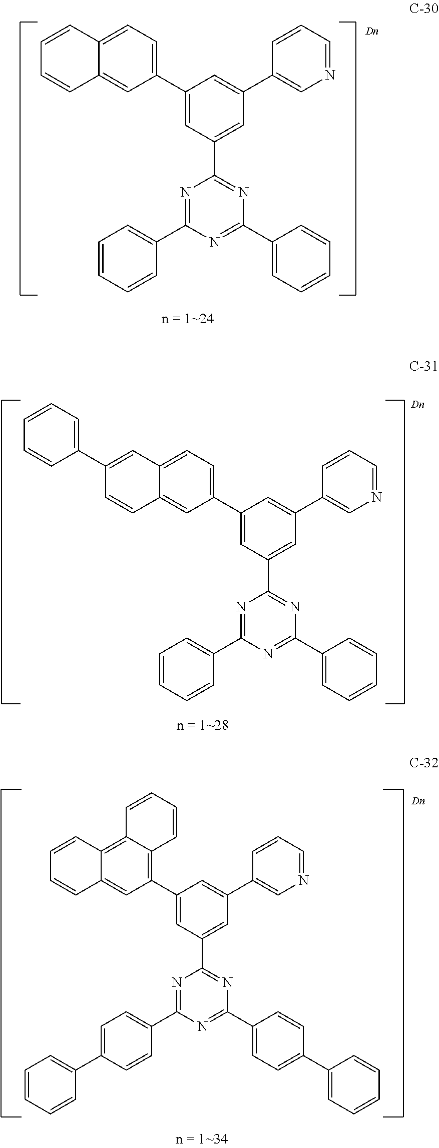 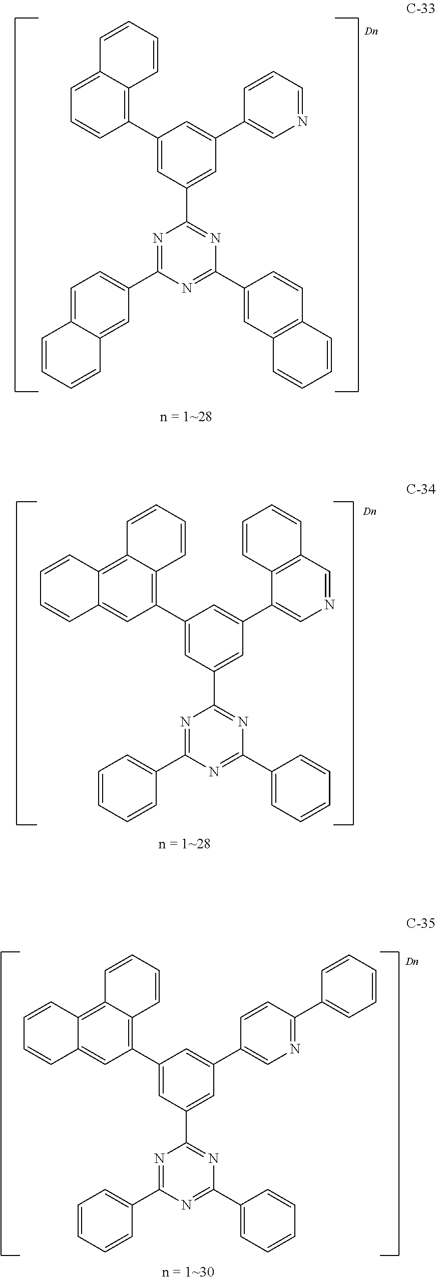 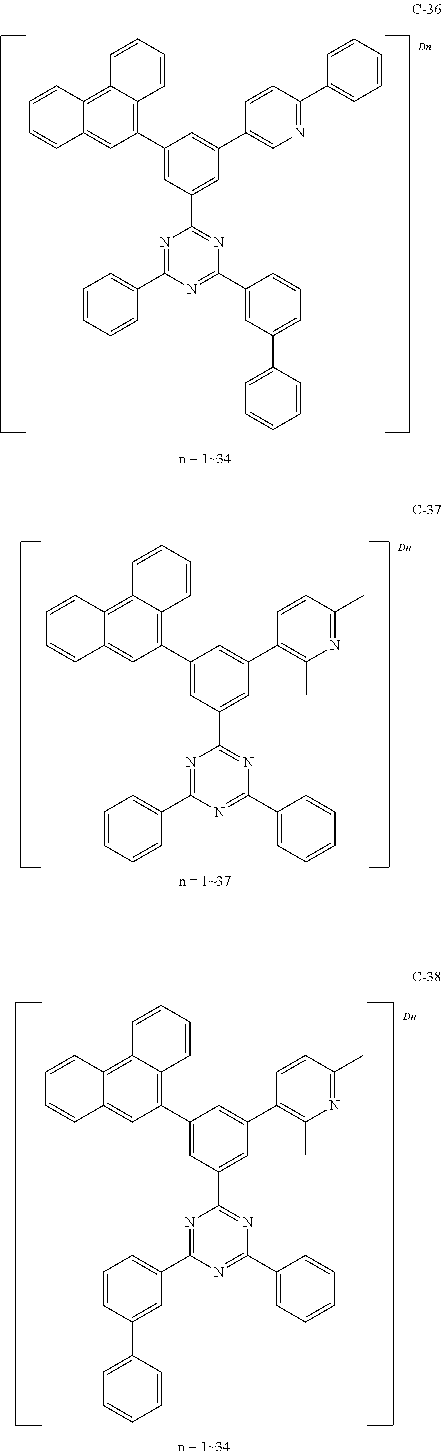 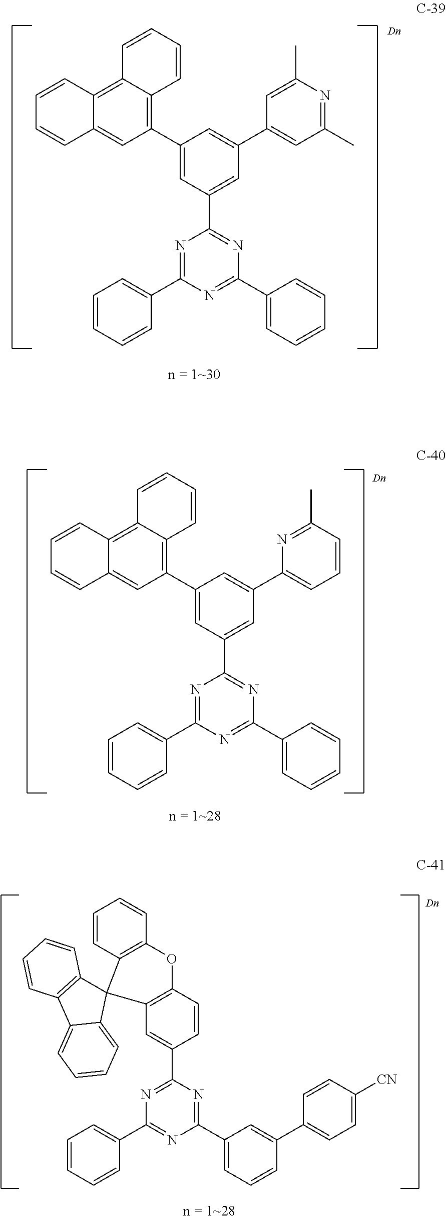 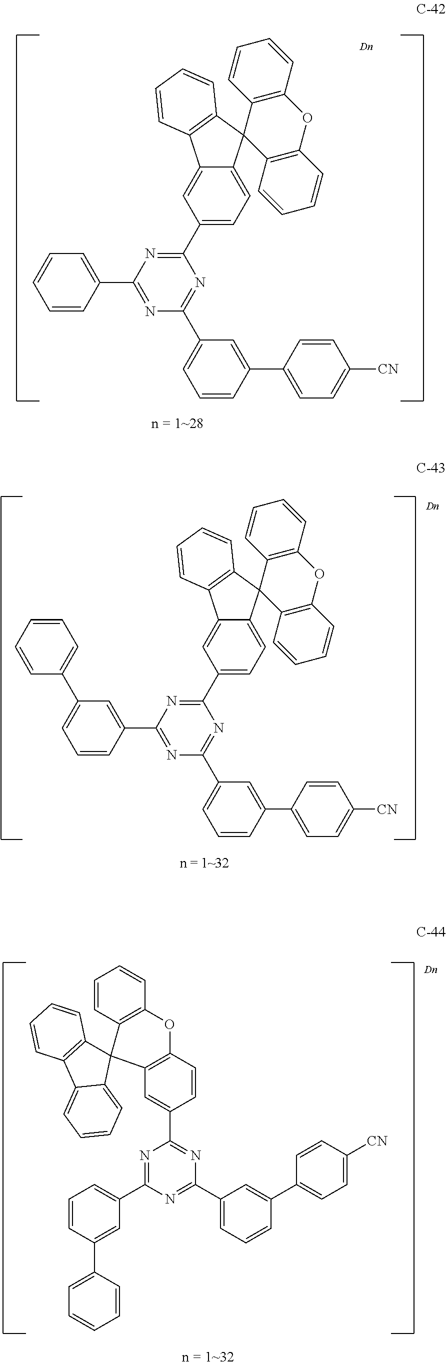 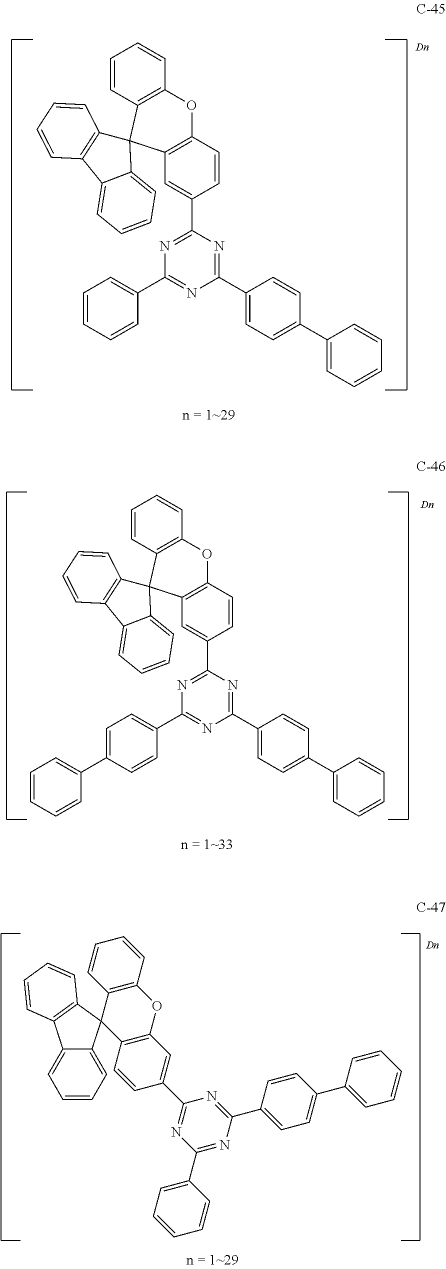 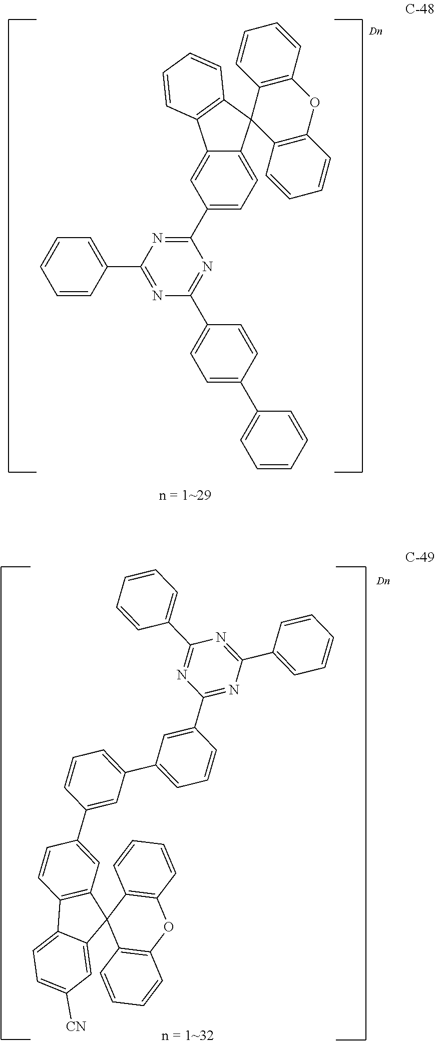 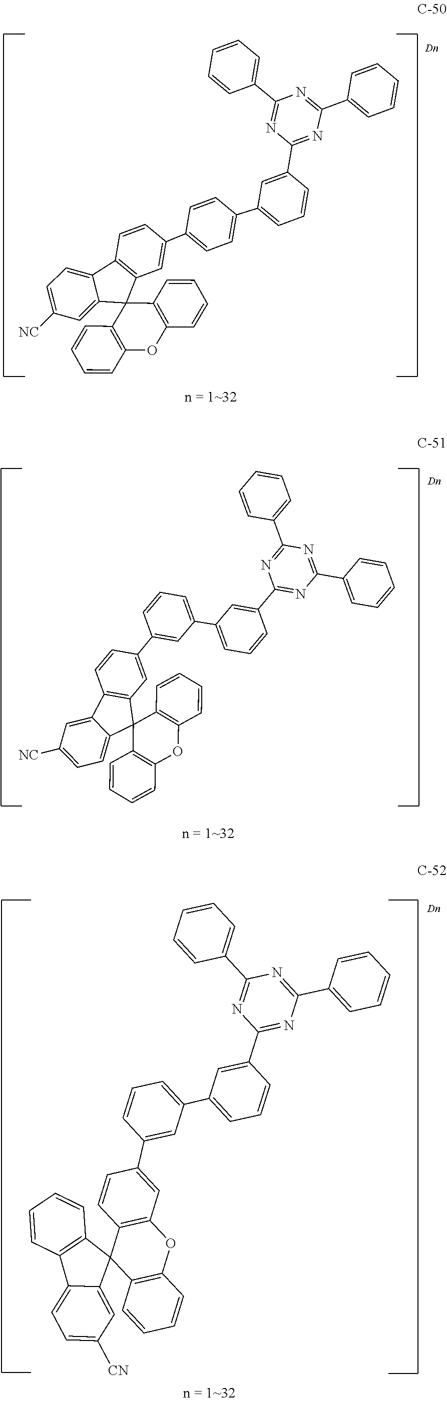 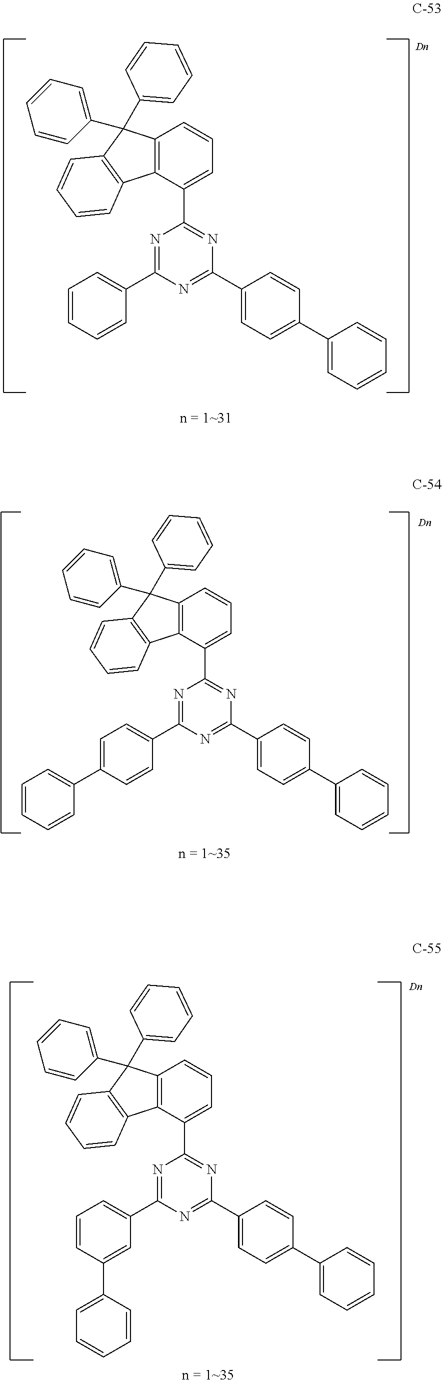 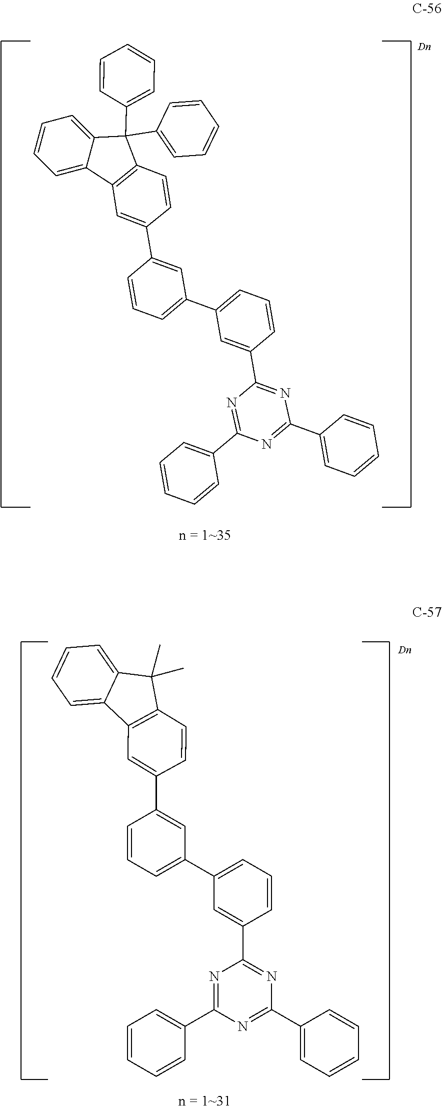 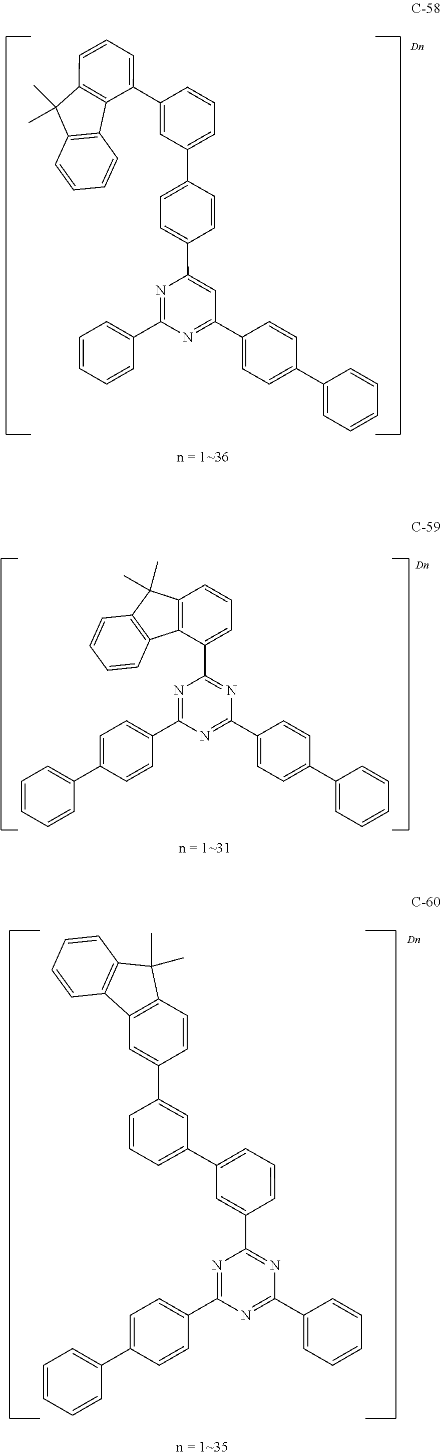 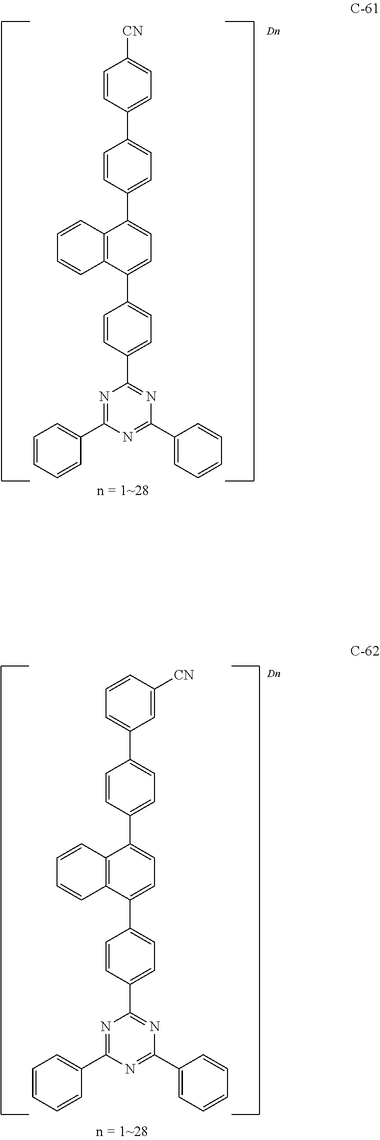 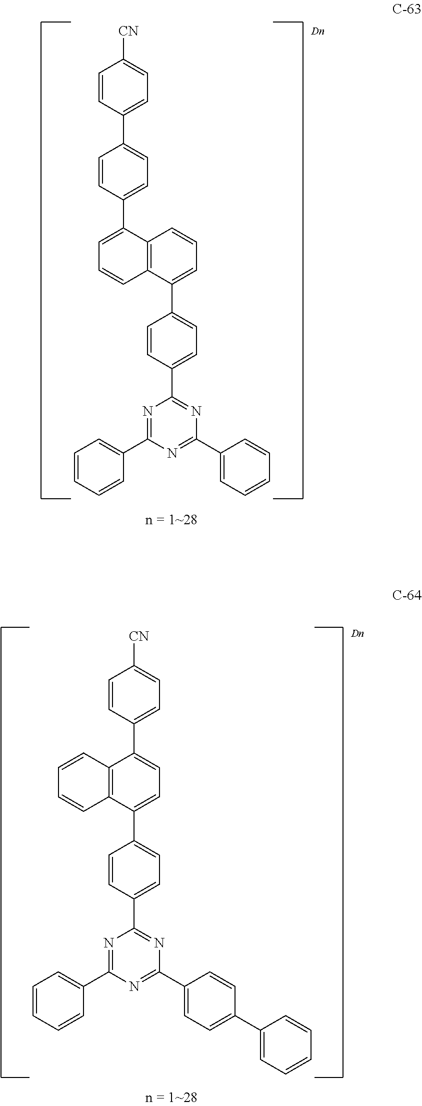 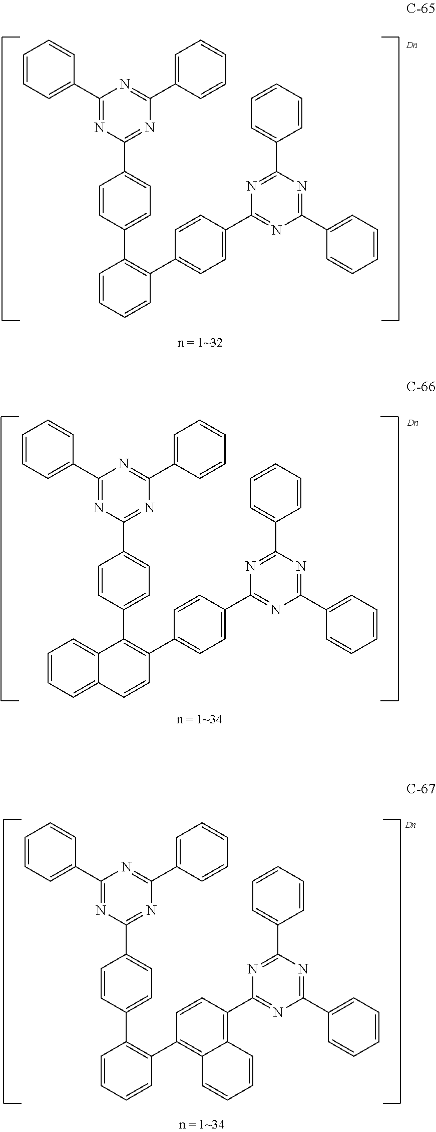 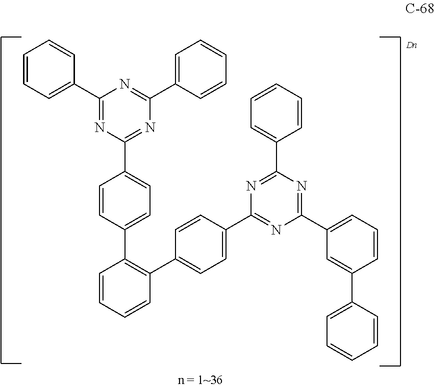
wherein Dn means that n number of hydrogens is replaced with deuterium, wherein the upper limit of n is determined by the number of hydrogens that can be substituted in each compound.

11. The organic electroluminescent device according to claim 1, wherein the organic layer includes a hole blocking layer, an electron transport layer, or an electron injection layer.

12. An organic electroluminescent device comprising
an anode;
a cathode;
at least one light-emitting layer positioned between the anode and the cathode; and
at least one organic layer positioned between the light-emitting layer and the cathode, wherein the organic layer includes at least one deuterated compound, and wherein the light-emitting layer includes a plurality of host materials including at least one first host compound and at least one second host compound.

13. The organic electroluminescent device according to claim 12, wherein at least one of the first host compound and the second host compound includes deuterium.

14. The organic electroluminescent device according to claim 12, wherein at least one of the first host compound and the second host compound contain an anthracene backbone, a triazine backbone, a dibenzofuran backbone, a dibenzothiophene backbone, or a carbazole backbone.
